(19) United States
(12) Patent Application Publication
(10) Pub. No.: US 2019/0233856 A1
(43) Pub. Date: Aug. 1, 2019

(54) CELLS AND METHOD FOR PRODUCING RHAMNOLIPIDS USING ALTERNATIVE GLUCOSE TRANSPORTERS

(71) Applicant: Evonik Degussa GmbH, Essen (DE)

(72) Inventors: Oliver Thum, Ratingen (DE); Steffen Schaffer, Herten (DE); Christoph Schorsch, Frankfurt am Main (DE); Mirja Wessel, Bochum (DE)

(73) Assignee: Evonik Degussa GmbH, Essen (DE)

(21) Appl. No.: 16/334,781

(22) PCT Filed: Oct. 18, 2017
(86) PCT No.: PCT/EP2017/076625

§ 371 Date: Mar. 20, 2019

(30) Foreign Application Priority Data
Oct. 24, 2016 (EP) 16195195.9

(51) Int. Cl. C12P 7/64; C12P 19/44; C12N 9/10; C12N 9/02; C07K 14/21; C12R 1/39; C12R 1/40; C12R 1/385

(52) U.S. Cl. CPC C12P 7/6463; C12P 19/44; C12N 9/1029; C12N 9/0036; C07K 14/21; C12Y 101/05002; C12R 1/40; C12R 1/385; C12Y 207/03009; C12Y 204/01159; C12R 1/39

(57) ABSTRACT

The invention relates to cells which make rhamnolipids and are genetically modified such that they have a decreased activity, compared to the wild type thereof, of an ABC glucose transporter and, compared to the wild type thereof, an increased activity of at least one non-ABC glucose transporter and to a method for producing rhamnolipids using the cells according to the invention.

### FIELD OF THE INVENTION

The invention relates to cells which make rhamnolipids and are genetically modified such that they have a decreased activity, compared to the wild type thereof, of an ABC glucose transporter and, compared to the wild type thereof, an increased activity of at least one non-ABC glucose transporter and to a method for producing rhamnolipids using the cells according to the invention.

### PRIOR ART

Rhamnolipids are a class of substances that is of economic interest, since they can potentially replace conventional petroleum-based surfactants and thus improve the environmental compatibility of the corresponding formulations. Rhamnolipids are nowadays produced using wild-type isolates of various human-pathogenic and animal-pathogenic bacteria, especially representatives of the genera Pseudomonas and Burkholderia (see Handbook of Hydrocarbon and Lipid Microbiology, 2010, pages 3037-51).

WO2012013554 discloses the production of rhamnolipids in non-pathogenic organisms such as, for example, P. putida KT2440, in which, for example, the enzymes encoded by the Pseudomonas aeruginosa genes rhlA, rhlB and rhlC are expressed.

The development and establishment of maximally efficient production methods requires, inter alia, the attainment of a highest possible space-time yield.

It is an object of the invention to develop microbial cells and methods using such cells in which a high space-time yield is achieved.

### DESCRIPTION OF THE INVENTION

It was found that, surprisingly, cells which make rhamnolipids and are genetically modified such that they have a decreased activity, compared to the wild type thereof, of an ABC glucose transporter and, compared to the wild type thereof, an increased activity of at least one non-ABC glucose transporter provide, in the case of comparable substrate input, a higher space-time yield of rhamnolipids than the comparative strains having a functional ABC transporter.

The present invention therefore provides rhamnolipid-making cells, characterized in that they are genetically modified such that they have a decreased activity, compared to the wild type thereof, of an ABC glucose transporter and an increased activity, compared to the wild type thereof, of at least one non-ABC glucose transporter.

The invention further provides a method for producing rhamnolipids using the aforementioned cells as biocatalyst.

One advantage of the present invention is that it is possible to use organisms which are not pathogenic and are easy to culture.

Another advantage of the present invention is that it is possible to make use of a large selection of carbon sources.

A further advantage is that it is not necessary in all circumstances to use oils as sole substrate or as co-substrate.

Another advantage is that it is possible with the aid of the invention to produce rhamnolipids having defined and modulatable properties.

A further advantage is that it is possible to produce rhamnolipids with higher space-time and carbon yields than with cells with no change in these activities.

The present invention therefore provides rhamnolipid-making cells, preferably isolated rhamnolipid-making cells, characterized in that they are genetically modified such that they have a decreased activity, compared to the wild type thereof, of an ABC glucose transporter and an increased activity, compared to the wild type thereof, of at least one non-ABC glucose transporter.

The term “wild type” of a cell denotes here a cell whose genome is present in a state as has arisen naturally by evolution. The term is used both for the whole cell and for individual genes. The term “wild type”, therefore, particularly does not include those cells or genes whose gene sequences have been at least partially modified by man by means of recombinant techniques. The term “wild type” denotes in particular the phenotype, the genotype or the gene that occurs most frequently in numbers in a natural population of organisms.

In the context of the present invention, the term “rhamnolipid” is understood to mean a compound of the general formula (I) or the salt thereof,

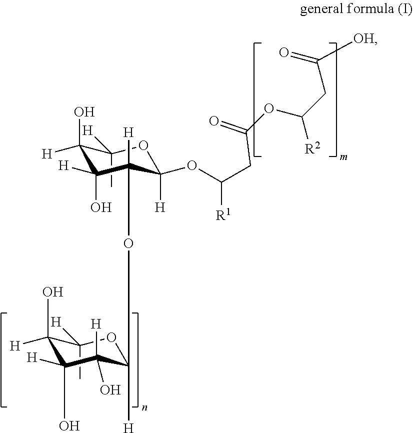

where

m=2, 1 or 0, in particular 1 or 0,

n=1 or 0, in particular 1,

R¹=organic radical having 2 to 24, preferably 5 to 13, carbon atoms, in particular optionally branched, optionally substituted, in particular hydroxy-substituted, optionally unsaturated, in particular optionally mono-, bi- or tri-unsaturated, alkyl radical, preferably one selected from the group consisting of pentenyl, heptenyl, nonenyl, undecenyl and tridecenyl and (CH₂)ₒ—CH₃ where o=1 to 23, preferably 4 to 12, and

R²=independently of one another, identical or different, organic radical having 2 to 24, preferably 5 to 13, carbon atoms, in particular optionally branched, optionally substituted, in particular hydroxy-substituted, optionally unsaturated, in particular optionally mono-, bi- or tri-unsaturated, alkyl radical, preferably one selected from the group consisting of pentenyl, heptenyl, nonenyl, undecenyl and tridecenyl and (CH₂)o-CH₃ where o=1 to 23, preferably 4 to 12.

If the cell according to the invention is able to make a rhamnolipid where m=1, it is preferred that the radical determined via R¹ and R² is

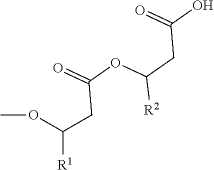

derived from 3-hydroxyoctanoyl-3-hydroxyoctanoic acid, 3-hydroxyoctanoyl-3-hydroxydecanoic acid, 3-hydroxy decanoyl-3-hydroxyoctanoic acid, 3-hydroxyoctanoyl-3-hydroxydecenoic acid, 3-hydroxy decenoyl-3-hydroxyoctanoic acid, 3-hydroxyoctanoyl-3-hydroxydodecanoic acid, 3-hydroxydodecanoyl-3-hydroxyoctanoic acid, 3-hydroxyoctanoyl-3-hydroxydodecenoic acid, 3-hydroxydodecenoyl-3-hydroxyoctanoic acid, 3-hydroxydecanoyl-3-hydroxydecanoic acid, 3-hydroxydecanoyl-3-hydroxydecenoic acid, 3-hydroxydecenoyl-3-hydroxydecanoic acid, 3-hydroxydecenoyl-3-hydroxydecenoic acid, 3-hydroxydecanoyl-3-hydroxydodecanoic acid, 3-hydroxydodecanoyl-3-hydroxydecanoic acid, 3-hydroxydecanoyl-3-hydroxydodecenoic acid, 3-hydroxydecanoyl-3-hydroxytetradecenoic acid, 3-hydroxytetradecanoyl-3-hydroxydecenoic acid, 3-hydroxydodecenoyl-3-hydroxydecanoic acid, 3-hydroxydecanoyl-3-hydroxytetradecanoic acid, 3-hydroxytetradecanoyl-3-hydroxydecanoic acid, 3-hydroxydecanoyl-3-hydroxytetradecenoic acid, 3-hydroxytetradecenoyl-3-hydroxydecanoic acid, 3-hydroxydodecanoyl-3-hydroxydodecanoic acid, 3-hydroxydodecenoyl-3-hydroxydodecanoic acid, 3-hydroxydodecanoyl-3-hydroxydodecenoic acid, 3-hydroxydodecanoyl-3-hydroxytetradecanoic acid, 3-hydroxytetradecanoyl-3-hydroxydodecanoic acid, 3-hydroxytetradecanoyl-3-hydroxytetradecanoic acid, 3-hydroxyhexadecanoyl-3-hydroxytetradecanoic acid, 3-hydroxytetradecanoyl-3-hydroxyhexadecanoic acid or 3-hydroxyhexadecanoyl-3-hydroxyhexadecanoic acid.

It is evident to a person skilled in the art that a cell according to the invention is also able to make mixtures of various rhamnolipids of the general formula (I).

In this connection, it is preferred that the cells according to the invention are able to make mixtures of rhamnolipids of the general formula (I), characterized in that n=1 in more than 80% by weight, preferably more than 90% by weight, particularly preferably more than 95% by weight, of the rhamnolipids made and the radical determined via R¹ and R² is derived from 3-hydroxydecanoyl-3-hydroxyoctanoic acid or 3-hydroxyoctanoyl-3-hydroxydecanoic acid in less than 20% by weight, preferably less than 15% by weight, of the rhamnolipids made, the specified % by weight being based on the sum of all rhamnolipids of the general formula (I) made.

The accession numbers listed in the context of the present invention correspond to the protein bank database entries of the NCBI with a date of 26 Jan. 2016; generally, in the present case, the version number of the entry is identified by “.number” such as, for example, “0.1”. Unless stated otherwise, all percentages (%) given are percentages by mass.

The expression “decreased activity of an enzyme Eₓ” used is accordingly understood to mean preferably activity decreased by a factor of at least 0.5, particularly preferably at least 0.1, further preferably at least 0.01, still further preferably at least 0.001 and most preferably at least 0.0001. The expression “decreased activity” also includes no detectable activity (“activity of zero”).

Methods for decreasing enzymatic activities in microorganisms are known to a person skilled in the art. Molecular biology techniques in particular are useful here. For example, the activity of a certain enzyme can be decreased by targeted mutation or by other measures known to a person skilled in the art for decreasing the activity of a certain enzyme. Instructions for modifying and decreasing protein expression and associated enzyme activity decrease specifically for Pseudomonas and Burkholderia, in particular for interrupting specific genes, can be found by a person skilled in the art in, for example, Dubeau et al. 2009. BMC Microbiology 9:263; Singh & Rohm. Microbiology. 2008. 154:797-809 or Lee et al. FEMS Microbiol Lett. 2009. 297(1):38-48. The preferred ways of decreasing the enzymatic activity of the ABC glucose transporter that are described below can similarly be preferably used for further enzyme activities to be decreased in the context of the present invention.

Cells preferred according to the invention are characterized in that the decrease in enzymatic activity is achieved by genetic modification of the gene encoding the ABC glucose transporter, said modification being selected from the group comprising, preferably consisting of, insertion of foreign DNA into the gene, deletion of at least parts of the gene, point mutations in the gene sequence, especially in or of regulatory sequences, such as, for instance, promoters and terminators or of ribosomal binding sites.

In this context, foreign DNA is understood to mean any DNA sequence which is “foreign” to the gene (and not to the organism), i.e. endogenous DNA sequences can also function as “foreign DNA” in this context. In this context, the gene is particularly preferably interrupted by insertion of a selection marker gene; the foreign DNA is therefore a selection marker gene, the insertion preferably having taken place by homologous recombination into the gene locus.

Cells alternatively preferred according to the invention are characterized in that the decrease in enzymatic activity is achieved by a targeted, transcriptional or post-transcriptional gene silencing of the gene encoding the ABC glucose transporter, especially with the aid of at least one repressor binding to the promoter of the gene encoding the ABC glucose transporter, by means of nonsense-mediated mRNA decay (NMD) and RNA interference (RNAi), with RNAi preferably making use of microRNA methodology (miRNA) or of the small interfering RNA method (siRNA), by means of which the mRNA of the ABC glucose transporter is degraded.

Cells according to the invention have been genetically modified such that they, compared to the wild type thereof, have a decreased activity of an ABC glucose transporter.

The term “ABC glucose transporter” in the context of the present invention is to be understood to mean membrane proteins which have an ATP-binding cassette (ABC) as a common structural element and transport specific substrates such as glucose actively across a cell membrane. ABC transporters consist of four core domains: two integral membrane domains and two cytoplasmic ATP-binding domains, the so-called ATP-binding cassettes. Said cassettes (functional domains/regions in a protein) are the basis for the energy-coupled transport of substrate against a concentration gradient.

In the context of the present invention, the activity of the ABC glucose transporter is defined as the ability to get 2-(N-(7-nitrobenz-2-oxa-1,3-diazol-4-yl)amino)-2-deoxyglucose (2-NBDG) (in place of glucose and measurable) into the cell.

The activity of the ABC glucose transporter can be determined with the aid of the Glucose Uptake Cell-Base Assay Kit, item No. 600470 from Cayman Chemicals, specifically in accordance with the manufacturer's instructions dated 9 Oct. 2015. It is clear to a reasonable person skilled in the art that, to this end, cells merely differing in the genetic modification directly directed towards the decrease in activity of the ABC glucose transporter are directly compared with one another in order to determine whether there is a difference in activity.

Cells according to the invention have been genetically modified such that they, compared to the wild type thereof, have an increased activity of at least one non-ABC glucose transporter.

The term “non-ABC glucose transporter” in the context of the present invention defines glucose transporters which are not ABC glucose transporters as per the definition above.

Particularly preferably, use is made here of non-ABC glucose transporters that are foreign to the cell according to the invention, therefore those that are not present in the wild-type genome.

Preferred non-ABC glucose transporters are selected in particular from the group consisting of phosphoenolpyruvate phosphotransferase systems of EC 2.7.3.9, galactose permeases, glucose facilitators, myo-inositol transporters, glucose permeases and glucose/galactose transporters.

Transporters are classified by a person skilled in the art in accordance with the Transporter Classification Database (www.tcdb.org). Preferred non-ABC glucose transporters in accordance with this classification in the context of the present invention are those selected from the TCDB families 2.A.1 Major Facilitator Superfamily, 2.A.123 The Seet Superfamily and 2.A.7 DMT Superfamily, and the ones that are particularly preferred are selected from the TCDB transporter classes 2.A.1.1.1, 2.A.1.1.4, 2.A.1.1.53, 2.A.1.7.3, 2.A.1.1.81, 2.A.123.2 and 2.A.7.5.

Preferred non-ABC glucose transporters are particularly selected from enzymes encoded by a galP, glf, iolT1, glcP, gluP, SemiSWEET or glcU gene and PTS systems (consisting of the components enzyme I, HPr, enzyme IIA, enzyme IIB and enzyme IIC, it being possible for enzymes IIA, IIB and IIC to be present as fusion proteins) or enzymes having a polypeptide sequence in which up to 25%, preferably up to 20%, particularly preferably up to 15%, in particular up to 10, 9, 8, 7, 6, 5, 4, 3, 2, 1%, of the amino acid residues of the galP, glf, iolT1, glcP, gluP, SemiSWEET or glcU gene-encoded enzymes and PTS systems are modified by deletion, insertion, substitution or a combination thereof and which still has at least 10%, preferably 50%, particularly preferably 80%, in particular more than 90%, of the enzymatic activity of the galP, glf, iolT1, glcP, gluP, SemiSWEET or glcU gene-encoded enzyme and PTS system, enzymatic activity for a non-ABC glucose transporter being understood to mean the ability to get 2-(N-(7-nitrobenz-2-oxa-1,3-diazol-4-yl)amino)-2-deoxyglucose (2-NBDG) into the cell.

For the aforementioned polypeptide sequences, the iolT1 gene is especially that from C. glutamicum, the glcP gene is especially one from M smegmatis, S. frigidimarina or S. amazonensis, the gluP gene is especially that from B. abortus, the SemiSWEET gene is that from L. biflexa and the glcU gene is especially one from B. subtilis or S. xylosus.

The activity of the non-ABC glucose transporter can be determined with the aid of the Glucose Uptake Cell-Base Assay Kit, item No. 600470 from Cayman Chemicals, specifically in accordance with the manufacturer's instructions dated 9 Oct. 2015.

The cells according to the invention can be prokaryotes or eukaryotes. They can be mammalian cells (such as human cells), plant cells or microorganisms such as yeasts, fungi or bacteria, with microorganisms being particularly preferred and bacteria and yeasts being most preferred.

Furthermore, it is advantageous according to the invention when the cell according to the invention is a cell which, as wild type, is able to make polyhydroxyalkanoates having chain lengths of the monoalkanoate of from C₆ to C₁₆. Such cells are, for example, Burkholderia sp., Burkholderia thailandensis, Pseudomonas sp., Pseudomonas putida, Pseudomonas aeruginosa, Pseudomonas oleovorans, Pseudomonas chlororaphis, Pseudomonas stutzeri, Pseudomonas fluorescens, Pseudomonas citronellolis, Pseudomonas resinovorans, Comamonas testosteroni, Aeromonas hydrophila, Cupriavidus necator, Alcaligenes latus and Ralstonia eutropha. In this context, preferred inventive cells are genetically modified such that they, compared to the wild type thereof, are able to make fewer polyhydroxyalkanoates.

Within the bacteria group, particular preference is given to, in particular, Pseudomonas putida, Escherichia coli and Burkholderia thailandensis.

The starting strains of the cells according to the invention can be natural rhamnolipid producers, those cells which already produce rhamnolipids as wild type, or cells in which rhamnolipid production has only been made possible by gene technology.

In both cases, cells preferred according to the invention benefit from the fact that they have been genetically modified such that they, compared to the wild type thereof, have an increased activity of at least one of the enzymes selected from the group E₁, E₂ and E₃, the enzyme E₁ being able to catalyse the conversion of 3-hydroxyalkanoyl-ACP via 3-hydroxyalkanoyl-3-hydroxyalkanoic acid-ACP to hydroxyalkanoyl-₃-hydroxyalkanoic acid, the enzyme E₂ being a rhamnosyltransferase I and being able to catalyse the conversion of dTDP-rhamnose and 3-hydroxyalkanoyl-3-hydroxyalkanoate to α-L-rhamnopyranosyl-3-hydroxyalkanoyl-3-hydroxyalkanoate, and the enzyme E₃ being a rhamnosyltransferase II and being able to catalyse the conversion of dTDP-rhamnose and α-L-rhamnopyranosyl-3-hydroxyalkanoyl-3-hydroxyalkanoate to α-L-rhamnopyranosyl-(1-2)-α-L-rhamnopyranosyl-3-hydroxyalkanoyl-3-hydroxyalkanoate.

Enzyme E₁ is preferably selected from enzymes which are encoded by an rhlA gene and also enzymes having a polypeptide sequence in which up to 25%, preferably up to 20%, particularly preferably up to 15%, in particular up to 10, 9, 8, 7, 6, 5, 4, 3, 2, 1%, of the amino acid residues are modified with respect to the enzymes encoded by an rhlA gene by deletion, insertion, substitution or a combination thereof and which still has at least 10%, preferably 50%, particularly preferably 80%, in particular more than 90%, of the enzymatic activity of the enzyme having the reference sequence of the enzymes encoded by an rhlA gene.

Enzyme E₂ is preferably selected from enzymes which are encoded by an rhlB gene and also enzymes having a polypeptide sequence in which up to 25%, preferably up to 20%, particularly preferably up to 15%, in particular up to 10, 9, 8, 7, 6, 5, 4, 3, 2, 1%, of the amino acid residues are modified with respect to the enzymes encoded by an rhlB gene by deletion, insertion, substitution or a combination thereof and which still has at least 10%, preferably 50%, particularly preferably 80%, in particular more than 90%, of the enzymatic activity of the enzyme having the reference sequence of the enzymes encoded by an rhlB gene.

Enzyme E₃ is preferably selected from enzymes which are encoded by an rhlC gene and also enzymes having a polypeptide sequence in which up to 25%, preferably up to 20%, particularly preferably up to 15%, in particular up to 10, 9, 8, 7, 6, 5, 4, 3, 2, 1%, of the amino acid residues are modified with respect to the enzymes encoded by an rhlC gene by deletion, insertion, substitution or a combination thereof and which still has at least 10%, preferably 50%, particularly preferably 80%, in particular more than 90%, of the enzymatic activity of the enzyme having the reference sequence of the enzymes encoded by an rhlC gene.

What is particularly preferred for E₁, E₂ and E₃:

enzyme E₁ is selected from the group consisting of,

at least one enzyme E₁ₐ having polypeptide sequence ADP06387.1, or having a polypeptide sequence in which up to 25%, preferably up to 20%, particularly preferably up to 15%, in particular up to 10, 9, 8, 7, 6, 5, 4, 3, 2, 1%, of the amino acid residues are modified with respect to the reference sequence ADP06387.1 by deletion, insertion, substitution or a combination thereof and which still has at least 10%, preferably 50%, particularly preferably 80%, in particular more than 90%, of the enzymatic activity of the enzyme having the reference sequence ADP06387.1, enzymatic activity for an enzyme E₁ₐ being understood to mean the ability to convert 3-hydroxydecanoyl-ACP via 3-hydroxydecanoyl-3-hydroxydecanoic acid-ACP to hydroxydecanoyl-3-hydroxydecanoic acid, at least one enzyme E_{1b} having polypeptide sequence AIP29471.1, CBI71021.1, NP_252169.1, ABR81106.1, YP_439272.1, YP_111362.1, YP_110557.1, YP_105231.1, ZP_02461688.1, ZP_02358949.1, ZP_01769192.1, ZP_04893165.1, ZP_02265387.2, ZP_02511781.1, ZP_03456835.1, ZP_03794633.1, YP_990329.1, ZP_02408727.1, YP_002908243.1, ZP_04884056.1, YP_004348703.1, ZP_04905334.1, ZP_02376540.1, EGC99875.1, ZP_02907621.1, YP_001811696.1, ZP_02466678.1, ZP_02891475.1, YP_776393.1, YP_002234939.1, YP_001778804.1, YP_371314.1, ZP_04943305.1, YP_623139.1, ZP_02417235.1 or ZP_04892059.1 or having a polypeptide sequence in which up to 25%, preferably up to 20%, particularly preferably up to 15%, in particular up to 10, 9, 8, 7, 6, 5, 4, 3, 2, 1%, of the amino acid residues are modified with respect to the particular aforementioned accession number by deletion, insertion, substitution or a combination thereof and which still has at least 10%, preferably 50%, particularly preferably 80%, in particular more than 90%, of the enzymatic activity of the enzyme having the particular aforementioned accession number, enzymatic activity for an enzyme E_{1b} being understood to mean the ability to convert 3-hydroxytetradecanoyl-ACP via 3-hydroxytetradecanoyl-3-hydroxytetradecanoic acid-ACP to hydroxytetradecanoyl-3-hydroxytetradecanoic acid,

enzyme E₂ is selected from the group consisting of,

at least one enzyme E₂a having polypeptide sequence ADP06388.1, YP_001347032.1, CB171029.1, YP_002439138.1, CBI71031.1, NP_252168.1, CBI71034.1, CBI71028.1, AAA62129.1 or ZP_04929750.1 or having a polypeptide sequence in which up to 25%, preferably up to 20%, particularly preferably up to 15%, in particular up to 10, 9, 8, 7, 6, 5, 4, 3, 2, 1%, of the amino acid residues are modified with respect to the particular aforementioned accession number by deletion, insertion, substitution or a combination thereof and which still has at least 10%, preferably 50%, particularly preferably 80%, in particular more than 90%, of the enzymatic activity of the enzyme having the particular aforementioned accession number, enzymatic activity for an enzyme E₂ₐbeing understood to mean the ability to convert dTDP-rhamnose and 3-hydroxydecanoyl-3-hydroxydecanoic acid to α-L-rhamnopyranosyl-3-hydroxydecanoyl-3-hydroxydecanoic acid,

at least one enzyme E_{2b} having polypeptide sequence AJY01590.1, ABR84881.1, NP_252168.1, FN601364.1, YP_440074.1, ZP_05590657.1, ZP_04520374.1, ZP_00438360.2, ZP_00438209.2, YP_001074761.1, ZP_04811084.1, YP_110558.1, YP_111361.1, ZP_02492857.1, YP_337246.1, YP_001061811.1, YP_105607.1, ZP_02371503.1, ZP_02503962.1, ZP_03456839.1, ZP_02461690.1, ZP_03794634.1, ZP_01769736.1, ZP_01769308.1, ZP_02358948.1, ZP_02487736.1, ZP_02408758.1, YP_002234937.1, ZP_02891477.1, YP_001778806.1, YP_623141.1, YP_838721.1, ZP_04943307.1, YP_776391.1, YP_004348704.1, ZP_02907619.1, YP_371316.1, ZP_02389948.1, YP_001811694.1, YP_002908244.1, ZP_02511808.1, ZP_02376542.1, EGC99877.1, ZP_02451760.1 or ZP_02414414.1 or having a polypeptide sequence in which up to 25%, preferably up to 20%, particularly preferably up to 15%, in particular up to 10, 9, 8, 7, 6, 5, 4, 3, 2, 1%, of the amino acid residues are modified with respect to the particular aforementioned accession number by deletion, insertion, substitution or a combination thereof and which still has at least 10%, preferably 50%, particularly preferably 80%, in particular more than 90%, of the enzymatic activity of the enzyme having the particular aforementioned accession number, enzymatic activity for an enzyme En being understood to mean the ability to convert dTDP-rhamnose and 3-hydroxytetradecanoyl-3-hydroxytetradecanoic acid to α-L-rhamnopyranosyl-3-hydroxytetradecanoyl-3-hydroxytetradecanoic acid, and

enzyme E₃ is selected from the group consisting of,

at least one enzyme E₃ₐ having polypeptide sequence NP 249821.1 or having a polypeptide sequence in which up to 25%, preferably up to 20%, particularly preferably up to 15%, in particular up to 10, 9, 8, 7, 6, 5, 4, 3, 2, 1%, of the amino acid residues are modified with respect to the reference sequence NP_249821.1 by deletion, insertion, substitution or a combination thereof and which still has at least 10%, preferably 50%, particularly preferably 80%, in particular more than 90%, of the enzymatic activity of the enzyme having the reference sequence NP_249821.1, enzymatic activity for an enzyme E₃ₐ being understood to mean the ability to convert dTDP-rhamnose and α-L-rhamnopyranosyl-3-hydroxydecanoyl-3-hydroxydecanoic acid to α-L-rhamnopyranosyl-(1-2)-α-L-rhamnopyranosyl-3-hydroxydecanoyl-3-hydroxydecanoic acid,

at least one enzyme E_{3b} having polypeptide sequence AJY02981.1, FN601387.1, FN601391.1 YP_440071.1, ZP_02375899.1, ZP_02466676.1, YP_001075863.1, ZP_02408796.1, YP_335530.1, ZP_01769176.1, YP_105609.1, ZP_01770867.1, ZP_04520873.1, YP_110560.1, YP_001024014.1, ZP_03450125.1, YP_001061813.1, YP_111359.1, ZP_00440994.2, ZP_03456926.1, ZP_02358946.1, ZP_00438001.2, ZP_02461478.1, ZP_02503929.1, ZP_02511832.1, YP_004348706.1, ZP_04898742.1, YP_002908246.1, ZP_02382844.1, EGD05167.1, YP_001778808.1, YP_001811692.1, YP_002234935.1, YP_371318.1, YP_623143.1, YP_776389.1, ZP_02891479.1, ZP_02907617.1, ZP_02417424.1 or ZP_04898743.1 or having a polypeptide sequence in which up to 25%, preferably up to 20%, particularly preferably up to 15%, in particular up to 10, 9, 8, 7, 6, 5, 4, 3, 2, 1%, of the amino acid residues are modified with respect to the particular aforementioned accession number by deletion, insertion, substitution or a combination thereof and which still has at least 10%, preferably 50%, particularly preferably 80%, in particular more than 90%, of the enzymatic activity of the enzyme having the aforementioned accession number, enzymatic activity for an enzyme E_{3b} being understood to mean the ability to convert dTDP-rhamnose and α-L-rhamnopyranosyl-3-hydroxytetradecanoyl-3-hydroxytetradecanoic acid to α-L-rhamnopyranosyl-(1-2)-α-L-rhamnopyranosyl-3-hydroxytetradecanoyl-3-hydroxytetradecanoic acid.

It is clear that the activities specifically indicated above for the enzymes E₁ₐ to E_{3b} are only a specific exemplary selection of a broader activity spectrum of the aforementioned enzymes; the activity mentioned in each case is that for which a reliable measurement method is available for a given enzyme. Thus, it is clear that an enzyme which converts a substrate having an unbranched, saturated C₁₀-alkyl radical will likewise convert—although possibly with reduced activity—those substrates having a C₆- or C₁₆-alkyl radical, which may possibly also be branched or unsaturated.

Cells preferred according to the invention are able, as wild type, to make no quantities or no detectable quantities of rhamnolipids and, furthermore, preferably have, as wild type, no activity or no detectable activity of the enzymes E₁, E₂ and E₃.

According to the invention, preference is given to cells which have increased activities of the following enzyme combinations: E₁, E₂, E₃, E₁E₂, E₁E₃, E₂E₃ and E₁E₂E₃, of which the combination

E₂, E₂E₃ and E₁E₂E₃, in particular E₁E₂E₃ is particularly preferred.

In a preferred embodiment of the cell according to the invention having an increased activity of the enzyme combination E₁E₂E₃, n is preferably=1.

In the context of the present invention, the term “increased activity of an enzyme” is preferably to be understood to mean an increased intracellular activity.

In principle, an increase in the enzymatic activity can be achieved by increasing the copy number of the gene sequence(s) coding for the enzyme, by using a strong promoter or an improved ribosome binding site, by attenuating negative regulation of gene expression, for example using transcription regulators, or by enhancing positive regulation of gene expression, for example using transcription regulators, by altering the codon usage of the gene, by increasing in various ways the half-life of the mRNA or of the enzyme, by modifying the regulation of expression of the gene or by using a gene or allele coding for a corresponding enzyme with increased activity and by combining these measures as appropriate. The increase in the activity is preferably increased according to the invention by increasing the copy number of the gene sequence, which codes for the enzyme, in comparison to the wild type. The incorporation of a copy of a gene sequence, which was not previously present in the wild type, self-evidently corresponds to an increase in the copy number from 0 to 1.

Cells genetically modified according to the invention are generated, for example, by transformation, transduction, conjugation, or a combination of these methods, with a vector containing the desired gene, an allele of this gene or parts thereof and optionally a promoter enabling the gene to be expressed. Heterologous expression is achieved in particular by integrating the gene or alleles into the chromosome of the cell or an extrachromosomally replicating vector.

An overview of the options for increasing enzyme activity in cells is given for pyruvate carboxylase by way of example in DE-A-100 31 999, which is hereby incorporated by way of reference and whose disclosure forms part of the disclosure of the present invention regarding the options for increasing enzyme activity in cells.

Expression of the enzymes or genes specified above and all enzymes or genes specified below is detectable with the aid of 1- and 2-dimensional protein gel separation and subsequent optical identification of the protein concentration in the gel using appropriate evaluation software. If the increase in an enzyme activity is based exclusively on an increase in expression of the corresponding gene, the increase in said enzyme activity can be quantified in a simple manner by comparing the 1- or 2-dimensional protein separations between wild type and genetically modified cell. A customary method of preparing protein gels in the case of coryneform bacteria and of identifying said proteins is the procedure described by Hermann et al. (Electrophoresis, 22: 1712.23 (2001)). Protein concentration can likewise be analysed by Western blot hybridization using an antibody specific for the protein to be detected (Sambrook et al., Molecular Cloning: a laboratory manual, 2nd Ed. Cold Spring Harbor Laboratory Press, Cold Spring Harbor, N.Y. USA, 1989) and subsequent optical evaluation using appropriate software for determination of concentration (Lohaus and Meyer (1989) Biospektrum, 5: 32-39; Lottspeich (1999) Angewandte Chemie 111: 2630-2647). The activity of DNA-binding proteins can be measured by means of DNA band shift assays (also referred to as gel retardation) (Wilson et al. (2001) Journal of Bacteriology, 183: 2151-2155). The effect of DNA-binding proteins on the expression of other genes can be detected by various well-described reporter gene assay methods (Sambrook et al., Molecular Cloning: a laboratory manual, 2nd Ed. Cold Spring Harbor Laboratory Press, Cold Spring Harbor, N.Y. USA, 1989). Intracellular enzymatic activities can be determined by various described methods (Donahue et al. (2000) Journal of Bacteriology 182 (19): 5624-5627; Ray et al. (2000) Journal of Bacteriology 182 (8): 2277-2284; Freedberg et al. (1973) Journal of Bacteriology 115 (3): 816-823). If no specific methods for determining the activity of a particular enzyme are stated in the explanations below, the increase in enzyme activity and also the decrease in an enzyme activity are preferably determined by means of the methods described in Hermann et al., Electophoresis, 22: 1712-23 (2001), Lohaus et al., Biospektrum 5 32-39 (1998), Lottspeich, Angewandte Chemie 111: 2630-2647 (1999) and Wilson et al., Journal of Bacteriology 183: 2151-2155 (2001).

If the increase in the enzyme activity is accomplished by mutation of the endogenous gene, such mutations can either be generated in a non-directed manner according to classical methods, for example by UV radiation or by chemicals which cause mutation, or specifically by means of genetic engineering methods such as deletion(s), insertion(s) and/or nucleotide substitution(s).

Modified cells are obtained by these mutations. Particularly preferred mutants of enzymes are also particularly those enzymes which are no longer subject to feedback, product or substrate inhibition, or at least less so compared to the wild type enzyme.

If the increase in the enzyme activity is accomplished by increasing the synthesis of an enzyme, the copy number of the relevant genes, for example, is increased or the promoter and regulatory region or the ribosomal binding site, which is located upstream of the structural gene, is mutated. Expression cassettes which are incorporated upstream of the structural gene have a similar effect. Additionally, by means of inducible promoters, it is possible to increase expression at any desired time. Furthermore, however, so-called “enhancers” can also be assigned to the enzyme gene as regulatory sequences, which likewise cause increased gene expression via improved interaction between RNA polymerase and DNA. Expression is also improved by measures to prolong the lifetime of the mRNA. Moreover, enzyme activity is also intensified by preventing the degradation of the enzyme protein. Here, the genes or gene constructs are present either in plasmids of different copy number or are integrated in the chromosome and amplified. Alternatively, moreover, overexpression of the relevant genes can be achieved by modification of the medium composition and culturing. Instructions in relation thereto can be found by a person skilled in the art in, inter alia, Martin et al. (Bio/Technology 5, 137-146 (1987)), in Guerrero et al. (Gene 138, 35-41 (1994)), Tsuchiya and Morinaga (Bio/Technology 6, 428-430 (1988)), in Eikmanns et al. (Gene 102, 93-98 (1991)), in EP-A-0 472 869, in U.S. Pat. No. 4,601,893, in Schwarzer and Pühler (Bio/Technology 9, 84-87 (1991)), in Reinscheid et al. (Applied and Environmental Microbiology 60, 126-132 (1994)), in LaBarre et al. (Journal of Bacteriology 175, 1001-1007 (1993)), in WO-A-96/15246, in Malumbres et al. (Gene 134, 15-24 (1993)), in JP-A-10-229891, in Jensen and Hammer (Biotechnology and Bioengineering 58, 191-195 (1998)) and in known genetics and molecular biology textbooks. The measures described above, like the mutations, also result in genetically modified cells.

To increase the expression of the particular genes, episomal plasmids, for example, are used. In principle, as plasmids or vectors, all embodiments available to those skilled in the art for this purpose are possible. Such plasmids and vectors can, for example, be inferred from the brochures of Novagen, Promega, New England Biolabs, Clontech or Gibco BRL. Further preferred plasmids and vectors can be found in: Glover, D. M. (1985) DNA cloning: a practical approach, Vol. I-III, IRL Press Ltd., Oxford; Rodriguez, R. L. and Denhardt, D. T (eds) (1988) Vectors: a survey of molecular cloning vectors and their uses, 179-204, Butterworth, Stoneham; Goeddel, D. V. (1990) Systems for heterologous gene expression, Methods Enzymol. 185, 3-7; Sambrook, J.; Fritsch, E. F. and Maniatis, T. (1989), Molecular cloning: a laboratory manual, 2nd ed., Cold Spring Harbor Laboratory Press, New York.

The plasmid vector which contains the gene to be amplified is then transferred into the desired strain by conjugation or transformation. The method of conjugation is described, for example, in Schäfer et al., Applied and Environmental Microbiology 60: 756-759 (1994). Methods for transformation are described, for example, in Thierbach et al., Applied Microbiology and Biotechnology 29: 356-362 (1988), Dunican and Shivnan, Bio/Technology 7: 1067-1070 (1989) and Tauch et al., FEMS Microbiology Letters 123: 343-347 (1994). After homologous recombination by means of a “cross-over” event, the resulting strain comprises at least two copies of the gene concerned.

In the context of the present invention, the increase in the activity of an enzyme is achieved particularly preferably by an increase, compared to the wild-type cell, in the copy number of the region encoding the enzyme considered, especially in conjunction with a strong promoter, and, in the case of enzymes already present in the wild type, by using a stronger promoter compared to the one present in the wild-type gene.

The wording “an increased activity, compared to the wild type thereof, of an enzyme Eₓ” used above and in the explanations below should preferably always be understood to mean an activity of the particular enzyme Eₓ increased by a factor of at least 2, particularly preferably at least 10, further preferably at least 100, still further preferably at least 1,000 and most preferably at least 10,000. Furthermore, the cell according to the invention which has “an increased activity, compared to the wild type thereof, of an enzyme Eₓ” in particular also includes a cell, the wild type of which has no or at least no detectable activity of this enzyme Eₓ, and which only displays detectable activity of this enzyme Eₓ after increasing the enzyme activity, for example, by overexpression. In this context, the term “overexpression” or the wording “increase in expression” used in the explanations below also includes the case that a starting cell, for example a wild-type cell, displays no or at least no detectable expression and detectable synthesis of the enzyme Eₓ is only induced by recombinant methods.

Modifications of amino acid residues of a given polypeptide sequence which do not lead to a significant change in the properties and the function of the given polypeptide are known to the person skilled in the art. Thus, it is possible, for example, to interchange conserved amino acids; examples of such suitable amino acid substitutions are: Ala with Ser; Arg with Lys; Asn with Gln or His; Asp with Glu; Cys with Ser; Gln with Asn; Glu with Asp; Gly with Pro; His with Asn or Gln; Ile with Leu or Val; Leu with Met or Val; Lys with Arg or Gln or Glu; Met with Leu or Ile; Phe with Met or Leu or Tyr; Ser with Thr; Thr with Ser; Trp with Tyr; Tyr with Trp or Phe; Val with Ile or Leu. It is also known that modifications in particular at the N or C terminus of a polypeptide in the form of, for example, amino acid insertions or deletions frequently do not have a significant influence on the function of the polypeptide.

The “amino acid identity” in connection with the enzymes used in the context of the invention is determined with the aid of known methods. In general, use is made of special computer programs with algorithms taking into account specific requirements.

Preferred methods for determining the identity initially generate the greatest alignment between the sequences to be compared. Computer programs for determining the identity include, but are not limited to, the GCG program package including GAP (Deveroy, J. et al., Nucleic Acid Research 12 (1984), page 387), Genetics Computer Group University of Wisconsin, Medicine (Wi), and BLASTP, BLASTN and FASTA (Altschul, S. et al., Journal of Molecular Biology 215 (1990), pages 403-410). The BLAST program can be obtained from the National Center For Biotechnology Information (NCBI) and from other sources (BLAST Handbuch, Altschul S. et al., NCBI NLM NIH Bethesda N.D. 22894; Altschul S. et al., above).

The known Smith-Waterman algorithm can likewise be used for determining the identities. Preferred parameters for determining the “amino acid identity” are, when using the BLASTP program (Altschul, S. et al., Journal of Molecular Biology 215 (1990), pages 403-410):

| | |
|---|---|
| Expect Threshold: | 10 |
| Word size: | 3 |
| Matrix: | BLOSUM62 |
| Gap costs: | Existence: 11; Extension: 1 |
| Compositional | Conditional compositional |
| adjustments: | score matrix adjustment |

The above parameters are the default parameters for amino acid sequence comparison. The GAP program is likewise suitable for use with the above parameters.

In the context of the present invention, an identity of 60% according to the above algorithm means 60% identity. The same applies to higher identities.

The activity of an enzyme can be determined by disrupting cells containing said activity in a manner known to a person skilled in the art, for example with the aid of a bead mill, a French press or an ultrasound disintegrator, and then removing intact cells, cell debris and disruption aids, such as glass beads for instance, by 10 minutes of centrifugation at 11 000×g and 4° C. Using the resulting cell-free crude extract, it is then possible to carry out enzyme assays with subsequent LC-ESI-MS detection of the products. Alternatively, the enzyme can be enriched or else purified to homogeneity in a manner known to a person skilled in the art by chromatographic methods (such as nickel-nitrilotriacetic acid affinity chromatography, streptavidin affinity chromatography, gel-filtration chromatography or ion-exchange chromatography).

It is trivial and only mentioned for the sake of completeness that, to determine an activity increased or reduced compared to the wild type of a cell, a wild type reference culture is used which has been exposed to the same conditions as the sample to be determined.

The activity of the enzyme E₁ is determined using the cell-free crude extracts obtained as described above, as follows: A standard assay contains 100 μM E. coli ACP, 1 mM β-mercaptoethanol, 200 μM malonyl-coenzyme A, 40 μM octanoyl-coenzyme A (for E₁ₐ) or dodecanoyl-coenzyme A (for E_{1b}), 100 μM NADPH, 2 μg of E. coli FabD, 2 μg of Mycobacterium tuberculosis FabH, 1 μg of E. coli FabG, 0.1 M sodium phosphate buffer, pH 7.0, and 5 μg of enzyme E₅ in a final volume of 120 μL. ACP, β-mercaptoethanol and sodium phosphate buffer are pre-incubated at 37° C. for 30 min in order to reduce the ACP completely. The reaction is started by addition of enzyme E₁. The reactions are stopped using 2 ml of water which has been acidified to pH 2.0 using HCl and then extracted twice using 2 ml of chloroform/methanol (2:1 (v:v)). Phase separation is carried out by centrifugation (16 100 g, 5 min, RT). The lower organic phase is removed, fully evaporated in a vacuum centrifuge, and the sediment is taken up in 50 μl of methanol. Undissolved constituents are sedimented by centrifugation (16 100 g, 5 min, RT) and the sample analysed by means of LC-ESI-MS. The products are identified by analysis of the corresponding mass traces and of the MS² spectra.

The activity of the enzyme E₂ is then determined using the cell-free crude extracts obtained as described above, as follows: A standard assay can consist of 185 μl of 10 mM Tris-HCl (pH 7.5), 10 μl of 125 mM dTDP-rhamnose and 50 μl of crude protein extract (approximately 1 mg of total protein) or purified protein in solution (5 μg of purified protein). The reaction is started by the addition of 10 μl of 10 mM ethanolic solution of 3-hydroxydecanoyl-3-hydroxydecanoic acid (for E₂ₐ) or 3-hydroxytetradecanoyl-3-hydroxytetradecanoic acid (for E_{2b}) and incubated at 30° C. for 1 h with shaking (600 rpm). The reaction is then admixed with 1 ml of acetone. Undissolved constituents are sedimented by centrifugation (16 100 g, 5 min, RT) and the sample analysed by means of LC-ESI-MS. The products are identified by analysis of the corresponding mass traces and of the MS² spectra.

The activity of the enzyme E₃ is then determined using the cell-free crude extracts obtained as described above, as follows: A standard assay can consist of 185 μl of 10 mM Tris-HCl (pH 7.5), 10 μl of 125 mM dTDP-rhamnose and 50 μl of crude protein extract (approximately 1 mg of total protein) or purified protein in solution (5 μg of purified protein). The reaction is started by the addition of 10 μl of 10 mM ethanolic solution of α-L-rhamnopyranosyl-3-hydroxydecanoyl-3-hydroxydecanoic acid (for E₃a) or α-L-rhamnopyranosyl-3-hydroxytetradecanoyl-3-hydroxytetradecanoic acid (for E_{3b}) and incubated at 30° C. for 1 h with shaking (600 rpm). The reaction is then admixed with 1 ml of acetone. Undissolved constituents are sedimented by centrifugation (16 100 g, 5 min, RT) and the sample analysed by means of LC-ESI-MS. The products are identified by analysis of the corresponding mass traces and of the MS² spectra.

A cell preferred according to the invention is characterized in that it has been genetically modified such that it, compared to the wild type thereof, has a decreased activity of at least one enzyme E₄, which catalyses the conversion of D-glucose and quinone to D-glucono-1,5-lactone and quinol.

According to the invention, it is preferred that E₄ is a glucose 1-dehydrogenase of EC 1.1.5.2. Particularly preferred enzymes E₄ are selected from enzymes encoded by a gcd gene and also enzymes having a polypeptide sequence in which up to 25%, preferably up to 20%, particularly preferably up to 15%, in particular up to 10, 9, 8, 7, 6, 5, 4, 3, 2, 1%, of the amino acid residues are modified with respect to the enzymes encoded by a gcd gene by deletion, insertion, substitution or a combination thereof and which still has at least 10%, preferably 50%, particularly preferably 80%, in particular more than 90%, of the enzymatic activity of the enzyme having the reference sequence of the enzymes encoded by a gcd gene.

In particular, the enzymes E₄ are selected from enzymes E₄ having polypeptide sequence AAN67066.1 or having a polypeptide sequence in which up to 25%, preferably up to 20%, particularly preferably up to 15%, in particular up to 10, 9, 8, 7, 6, 5, 4, 3, 2, 1%, of the amino acid residues are modified with respect to AAN67066.1 by deletion, insertion, substitution or a combination thereof and which still has at least 10%, preferably 50%, particularly preferably 80%, in particular more than 90%, of the enzymatic activity of the enzyme having the reference sequence AAN67066.1.

The activity of an enzyme E₁ is determined using cell-free extracts with the aid of the Colorimetric Glucose Dehydrogenase Assay Kit from Abcam (Art.#ab102532) in accordance with the requirements of the manufacturer.

According to the invention, preference is given to cells which have modified activities of the following enzyme combinations:

E₄, E₁E₄, E₂E₄, E₃E₄, E₁E₂E₄, E₁E₃E₄, E₂E₃E₄ and E₁E₂E₃E₄, of which the combination

E₂E₄, E₂E₃E₄ and E₁E₂E₃E₄, in particular E₁E₂E₃E₄ is particularly preferred.

It is further advantageous and thus preferred when, additionally, the cell according to the invention has been genetically modified such that it, compared to the wild type thereof, has an increased activity of at least one enzyme E₅, which catalyses the export of a rhamnolipid of the general formula (I) from the cell into the surrounding medium.

In the case of cells preferred according to the invention, E₅ is selected from the group consisting of enzymes E₅ having polypeptide sequence AAG04520.1, AJY02996.1, ZP_05590661.1, YP_439278.1, YP_440069.1, ZP_04969301.1, ZP_04520234.1, YP_335528.1, YP_001075859.1, YP_001061817.1, ZP_02487499.1, YP_337251.1, ZP_04897712.1, ZP_04810190.1, YP_990322.1, ZP_02476924.1, ZP_04899735.1, ZP_04893873.1, ZP_02365982.1, YP_001062909.1, YP_105611.1, ZP_03794061.1, ZP_03457011.1, ZP_02385401.1, ZP_02370552.1, YP_105236.1, ZP_04905097.1, YP_776387.1, YP_001811690.1, YP_004348730.1, YP_004348708.1, YP_371320.1, YP_623145.1, YP_001778810.1, YP_002234933.1, CCE_{52909.1}, YP_002908248.1, ZP_04954557.1, ZP_04956038.1, ZP_02408950.1, ZP_02375897.1, ZP_02389908.1, YP_439274.1, YP_001074762.1, YP_337247.1, YP_110559.1, ZP_02495927.1, YP_111360.1, YP_105608.1, ZP_02487826.1, ZP_02358947.1, YP_001078605.1, ZP_00438000.1, ZP_00440993.1, ZP_02477260.1, YP_371317.1, YP_001778807.1, ZP_02382843.1, YP_002234936.1, YP_623142.1, ZP_02907618.1, ZP_02891478.1, YP_776390.1, ZP_04943308.1, YP_001811693.1, ZP_02503985.1, YP_004362740.1, YP_002908245.1, YP_004348705.1, ZP_02408798.1, ZP_02417250.1, EGD05166.1, ZP_02458677.1, ZP_02465793.1, YP_001578240.1, ZP_04944344.1, YP_771932.1, ZP_02889166.1, YP_002232614.1, ZP_03574808.1, ZP_02906105.1, YP_001806764.1, YP_619912.1, ,YP_001117913.1, YP_106647.1, YP_001763368.1, ZP_02479535.1, ZP_02461743.1, YP_560998.1, YP_331651.1, ZP_04893070.1, YP_003606714.1, ZP_02503995.1, ZP_06840428.1, YP_104288.1, ZP_02487849.1, ZP_02353848.1, YP_367475.1, ZP_02377399.1, ZP_02372143.1, YP_001897562.1, ZP_02361066.1, YP_440582.1, ZP_03268453.1, AET90544.1, YP_003908738.1, YP_004230049.1, ZP_02885418.1, CDH72316.1, WP_001297013.1, WP_010955775.1, WP_010955671.1, WP_010955672.1, WP_010955673.1, WP_010952401.1, WP_010952402.1, WP_010952403.1, WP_010952855.1, WP_010954573.1, WP_010954631.1, WP_010954632.1, WP_010954404.1, WP_004575310.1 or ZP_02511831.1 or having a polypeptide sequence in which up to 25%, preferably up to 20%, particularly preferably up to 15%, in particular up to 10, 9, 8, 7, 6, 5, 4, 3, 2, 1%, of the amino acid residues are modified with respect to the particular aforementioned accession number by deletion, insertion, substitution or a combination thereof and which still has at least 10%, preferably 50%, particularly preferably 80%, in particular more than 90%, of the enzymatic activity of the enzyme having the particular aforementioned accession number, enzymatic activity for an enzyme E₅ being understood to mean the ability to export a rhamnolipid of the general formula (I) from the cell into the surrounding medium.

The activity of the enzyme E₅ can then be determined using the cell-free crude extracts obtained as described above, by determining the amount of the enzyme E₅ made. This is based on the assumption that more enzyme E₅ per biomass unit is capable of exporting more rhamnolipid of the general formula (I) from the cell into the surrounding medium. Such a quantification can be carried out by immunological detection by means of antibodies specific for enzyme E₅ (see Kurien, T. B., Scofield, R. H (Eds.). Protein Blotting and Detection: Methods and Protocols. Methods in Molecular Biology, Vol. 536. 1st Ed., Humana Press. N.Y. USA, 2009) or by mass-spectrometry methods (see Schmidt, A., Kellermann, J. & Lottspeich, F. A novel strategy for quantitative proteornics using isotope-coded protein labels. Proteomics 5, 4-15 (2005)).

Alternatively, the activity of the enzyme E₅ can also be determined by carrying out uptake assays using radioactively labelled rhamnolipids and inside-out vesicles produced from the cells according to the invention. The general procedure is, for example, described in Nies DH. The cobalt, zinc, and cadmium efflux system CzcABC from Alcaligenes eutrophus functions as a cation-proton antiporter in Escherichia coli. J Bacteriol. 1995. 177(10):2707-12 or Lewinson O., Adler J, Poelarends G J, Mazurkiewicz P, Driessen A J, Bibi E. The Escherichia coli multidrug transporter MdfA catalyzes both electrogenic and electroneutral transport reactions.Proc Natl Acad Sci U S A. 2003 Feb. 18; 100(4):1667-72.

Cells according to the invention can be advantageously used for producing rhamnolipids. Therefore, the invention further provides for the use of cells according to the invention for producing compounds of the general formula (I).

The present invention further provides a method for producing rhamnolipids, especially those of the general formula (I),

where

m=2, 1 or 0, in particular 1 or 0,

n=1 or 0, in particular 1,

R¹and R²=mutually independently, identical or different, organic radical having 2 to 24, preferably 5 to 13, carbon atoms, in particular optionally branched, optionally substituted, particularly hydroxy-substituted, optionally unsaturated, in particular optionally mono-, bi- or tri-unsaturated, alkyl radical, preferably those selected from the group consisting of pentenyl, heptenyl, nonenyl, undecenyl and tridecenyl and (CH₂)ₒ-CH₃ where o=1 to 23, preferably 4 to 12,

comprising the process steps of

I) contacting the cell according to the invention with a medium containing a carbon source

II) culturing the cell under conditions allowing the cell to make rhamnolipid from the carbon source and

III) optionally isolating the rhamnolipids made.

The genetically modified cells according to the invention can be contacted with the culture medium and thus cultured in a continuous or discontinuous manner in a batch process or in a fed-batch process or repeated fed-batch process for the purposes of producing the aforementioned products. Also conceivable is a semi-continuous process, as described in GB-A-1009370. An overview of known cultivation methods is disclosed in the textbook by Chmiel (“Bioprozesstechnik 1. Einführung in die Bioverfahrenstechnik” [Bioprocess technology 1. Introduction to Bioprocess Technology] (Gustav Fischer Verlag, Stuttgart, 1991)) or in the textbook by Storhas (“Bioreaktoren and periphere Einrichtungen” [Bioreactors and Peripheral Devices] (Vieweg Verlag, Braunschweig/Wiesbaden, 1994)).

The culture medium to be used has to satisfy the demands of the particular strains in a suitable manner. Descriptions of culture media of various yeast strains are, for example, included in “Nonconventional yeast in biotechnology” (Ed. Klaus Wolf, Springer-Verlag Berlin, 1996).

The carbon source used can be carbohydrates such as, for example, glucose, sucrose, arabinose, xylose, lactose, fructose, maltose, molasses, starch, cellulose and hemicellulose, vegetable and animal oils and fats such as, for example, soya oil, safflower oil, arachis oil, hemp oil, jatropha oil, coconut fat, pumpkin seed oil, linseed oil, corn oil, poppy seed oil, evening primrose oil, olive oil, palm kernel oil, palm oil, rapeseed oil, sesame oil, sunflower oil, grape seed oil, walnut oil, wheatgerm oil and coconut fat, fatty acids, such as, for example, caprylic acid, capric acid, lauric acid, myristic acid, palmitic acid, palmitoleic acid, stearic acid, arachidonic acid, behenic acid, oleic acid, linoleic acid, linolenic acid, gamma-linolenic acid and the methyl or ethyl ester thereof and also fatty acid mixtures, mono-, di- and triglycerides containing the fatty acids just mentioned, alcohols such as, for example, glycerol, ethanol and methanol, hydrocarbons such as methane, carbonaceous gases and gas mixtures, such as CO, CO₂, synthesis or flue gas, amino acids such as L-glutamate or L-valine or organic acids such as, for example, acetic acid. These substances may be used individually or as a mixture. Particular preference is given to the use of carbohydrates, especially of monosaccharides, oligosaccharides or polysaccharides, as the carbon source, as described in U.S. Pat. No. 6,01,494 and U.S. Pat. No. 6,136,576, and of hydrocarbons, especially of alkanes, alkenes and alkynes and also the monocarboxylic acids derived therefrom and the mono-, di- and triglycerides derived from said monocarboxylic acids, and of glycerol and acetate. Very particular preference is given to mono-, di- and triglycerides containing the esterification products of glycerol with caprylic acid, capric acid, lauric acid, myristic acid, palmitic acid, palmitoleic acid, stearic acid, arachidonic acid, behenic acid, oleic acid, linoleic acid, linolenic acid and/or gamma-linolenic acid.

A major advantage of the present invention is that the cells according to the invention are able to make rhamnolipids from the simplest carbon sources such as, for example, glucose, sucrose or glycerol, meaning that it is not necessary to provide longer-chain carbon sources in the medium during the method according to the invention. Thus, in the event of insufficient availability, it is advantageous that the medium in step I) of the method according to the invention contains no amounts or no detectable amounts of carboxylic acids having a chain length of greater than six carbon atoms or esters or glycerides derivable therefrom.

The nitrogen source used may be organic nitrogen-containing compounds such as peptones, yeast extract, meat extract, malt extract, corn steep liquor, soya bean meal and urea or inorganic compounds such as ammonium sulphate, ammonium chloride, ammonium phosphate, ammonium carbonate and ammonium nitrate, ammonia, ammonium hydroxide or aqueous ammonia. The nitrogen sources may be used individually or as a mixture.

The phosphorus source used may be phosphoric acid, potassium dihydrogen phosphate or dipotassium hydrogen phosphate or the corresponding sodium-containing salts. Furthermore, the culture medium must contain salts of metals such as, for example, magnesium sulphate or iron sulphate that are necessary for growth. Finally, essential growth substances such as amino acids and vitamins may be used in addition to the substances mentioned above. Moreover, suitable precursors may be added to the culture medium. The aforementioned starting materials may be added to the culture in the form of a single batch or be appropriately fed in during cultivation.

To control the pH of the culture, appropriate use is made of basic compounds such as sodium hydroxide, potassium hydroxide, ammonia or aqueous ammonia or acidic compounds such as phosphoric acid or sulphuric acid. To control the evolution of foam, it is possible to use antifoams such as, for example, fatty acid polyglycol esters. To maintain the stability of plasmids, it is possible to add to the medium suitable selective substances such as, for example, antibiotics. In order to maintain aerobic conditions, oxygen or oxygenous gas mixtures, for example air, are introduced into the culture.

The temperature of the culture is normally more than 20° C., preferably more than 25° C., and it can also be more than 40° C., a cultivation temperature of 95° C., particularly preferably 90° C. and most preferably 80° C. advantageously not being exceeded.

In step III) of the method according to the invention, the rhamnolipids made by the cells can optionally be isolated from the cells and/or the culture medium, it being possible to use for the purposes of isolation all methods known to a person skilled in the art for isolating low-molecular-weight substances from complex compositions such as, for example, filtration, extraction, adsorption (chromatography) or crystallization.

Furthermore, the product phase contains remnants of biomass and various impurities, such as oils, fatty acids and other culture-medium constituents. The impurities are preferably removed in a solvent-free process. For example, the product phase can be diluted with water in order to facilitate pH adjustment. Product phase and aqueous phase can then be homogenized by transferring the rhamnolipids into a water-soluble form by lowering or raising the pH by means of acids or alkalis. Potentially, the solubilization of the rhamnolipids in the aqueous phase can be supported by incubation at relatively high temperatures, for example at from 60 to 90° C., and constant mixing. As a result of subsequent raising or lowering of the pH by means of alkalis or acids, the rhamnolipids can then be transferred into a water-insoluble form again, and so they can be easily separated from the aqueous phase. The product phase can then be additionally washed with water one or more times in order to remove water-soluble impurities.

Oil residues can, for example, be removed by extraction by means of suitable solvents, advantageously by means of organic solvents. An alkane such as, for example, n-hexane is preferred as solvent.

As an alternative to the above-described solvent-free process, the product can be removed from the aqueous phase using a suitable solvent, for example an ester such as, for example, ethyl acetate or butyl acetate. The stated extraction steps can be carried out in any desired order.

Here, solvents are preferably used, in particular organic solvents. The preferred solvent is n-pentanol. The solvent is removed by, for example, distillation. Thereafter, the lyophilized product can be further purified, for example by means of chromatographic methods. Examples which can be mentioned at this point include precipitation using suitable solvents, extraction using suitable solvents, complexing, for example by means of cyclodextrins or cyclodextrin derivatives, crystallization, purification or isolation by means of chromatographic methods or transfer of the rhamnolipids into easily removable derivatives.

A particularly suitable rhamnolipid isolation procedure in method step III) comprises the method sub steps of

A) transferring the rhamnolipids to an aqueous medium having a pH of less than 6,

B) contacting the medium with at least one organic solvent to obtain a multi-phase system and removing the aqueous phase,

C) increasing the pH to a pH of 6 or greater to obtain a multi-phase organic system,

D) removing an organic phase enriched with rhamnolipid and

E) optionally further purifying the rhamnolipid.

A detailed description of how to carry out this preferred embodiment of method step III) is given in US20140148588.

The present invention likewise provides the rhamnolipids obtainable using the method according to the invention, especially also the above-described rhamnolipid mixtures obtainable using the method according to the invention.

Advantageously, the rhamnolipids and mixtures obtainable using the method according to the invention can be used in cleaning agents, in cosmetic or pharmaceutical formulations and in crop-protection formulations.

Thus, the present invention further provides for the use of the rhamnolipids obtained using the method according to the invention for producing cosmetic, dermatological or pharmaceutical formulations, crop-protection formulations and also care products and cleaning agents and surfactant concentrates.

The examples adduced hereinafter describe the present invention by way of example, without any intention that the invention, the scope of application of which is apparent from the entirety of the description and the claims, be restricted to the embodiments specified in the examples.

### EXAMPLES

### Example 1 (Not Inventive)

Use was made of strain P. putida KT2440 αupp+pACYCATh5-{PrhaSR}[rhaSR_Ec]{PrhaBAD}[rhlABC_Pa]{Talk}[araC_Ec]{ParaBAD}[rmlBDAC_Pa]{Tal k}

Construction of the strain P. putida KT2440 Δupp+pACYCATh5-{PrhaSR}[rhaSR_Ec]{PrhaBAD}[rhlABC_Pa]{Talk}[araC_Ec]{ParaBAD}[rmlBDAC_Pa]{Tal k}

For the heterologous expression of the genes rhlA, rhlB and rhlC and of the genes rmlB, rmlD, rmlA and rmlC, both from P. aeruginosa, the plasmid pACYCATh5-{PrhaSR}[rhaSR_Ec]{PrhaBAD}[rhlABC_Pa]{Talk}[araC_Ec]{ParaBAD}[rmlBDAC_Pa]{Talk} was constructed. The plasmid contains, firstly, a synthetic operon consisting of the genes rhlA and rhlB (encoding a rhamnosyltransferase 1) and rhlC (encoding a rhamnosyltransferase 2) from P. aeruginosa DSM1128 (SEQ ID No 1) and, secondly, an operon consisting of the genes rmlB (encoding a dTDP-D-glucose 4,6-dehydratase), rmlD (encoding a dTDP-4-dehydrorhamnose reductase), rmlA (encoding a glucose-1-phosphate thymidylyltransferase) and rmlC (encoding a dTDP-4-dehydrorhamnose 3,5-epimerase) from P. aeruginosa DSM 19880 (SEQ ID No 2). The genes rhlABC are under the control of the rhamnose-inducible P_{Rha} promoter; the rmlBDAC genes are under the control of the arabinose-inducible P_{BAD} promoter. Situated downstream of the two operon structures is a terminator sequence (rrnB T1T2). The rmlBDAC genes were amplified from genomic DNA from P. aeruginosa DSM19880 and the synthetic rhlABC operon was obtained by gene synthesis. The P_{Rha} promoter cassette (SEQ ID No 3) and PBAD promoter cassette (SEQ ID No 4) and also the terminator sequence (SEQ ID No 5) were amplified from genomic E. coli DNA. Whereas the rhlABC genes are required for the synthesis of di-rhamnolipids, the rmlBDAC genes are needed for the provision of activated dTDP-L-rhamnose.

The vector is based on the plasmid pACYC184 (New England Biolabs, Frankfurt am Main, Germany) and bears a p15A origin of replication for replication in E. coli and a pVS1 origin of replication for replication in P. putida. The pVS1 origin of replication was amplified from the Pseudomonas plasmid pVS1 (Itoh Y, Watson J M, Haas D, Leisinger T, Plasmid 1984, 11(3), 206-20). The vector part and the DNA fragments were cloned using a commercially available in vitro DNA assembly kit (e.g. NEBuilder HiFi DNA Assembly Cloning Kit in accordance with the manufacturer's instructions (NEB; Frankfurt am Main, Germany)). Chemically competent E. coli 10 beta cells (NEB, Frankfurt am Main, Germany) were transformed in a manner known to a person skilled in the art. The correct insertion of the target genes was checked by restriction analysis and the authenticity of the introduced homologous regions confirmed by DNA sequencing. The size of the resulting plasmid pACYCATh5-{PrhaSR}[rhaSR_Ec]{PrhaBAD} [rhlABC_Pa]{Talk}[araC_Ec]{ParaBAD}[rmlBDAC_Pa]{Talk} (SEQ ID No 6) is 17 337 bp.

Thereafter, the plasmid was introduced into P. putida KT2440 Δupp. This strain is used as the starting strain for the construction of markerless gene deletions in P. putida (Graf & Altenbuchner, 2011, Applied and Environmental Microbiology, Vol 77, No. 15, 5549-5552, DOI:10.1128/AEM.05055-11). The method is based on a negative counter-selection system for P. putida, which utilizes the activity of uracil phosphoribosyltransferase and the sensitivity of P. putida towards the antimetabolite 5-fluorouracil. The deletion of the upp gene has no effect on rhamnolipid biosynthesis.

The transformation of P. putida KT2440 Δupp with the vector pACYCATh5-{PrhaSR}[rhaSR_Ec]{PrhaBAD}[rhlABC_Pa]{Talk}[araC_Ec]{ParaBAD}[rmlBDAC_Pa]{Talk} was carried out as described in Iwasaki et al. (Iwasaki K, Uchiyama H, Yagi O, Kurabayashi, T, Ishizuka K, Takamura Y, Biosci. Biotech. Biochem. 1994. 58(5):851-854). The plasmid DNA from each of 10 clones was isolated and analysed. A strain bearing the plasmid was called P. putida KT2440 Δupp pACYCATh5-{PrhaSR}[rhaSR_Ec]{PrhaBAD}[rhlABC_Pa]{Talk}[araC_Ec]{ParaBAD}[rmlBDAC Pa]{Talk}.

The biotechnological production of surfactant was carried out in the 8-fold parallel fermentation system “DASGIP” from Eppendorf.

For the fermentation, 1 L reactors were used. The pH probes were calibrated by means of a two-point calibration with measurement solutions of pH 4.0 and pH 7.0. The reactors were filled with 300 mL of water and autoclaved for 20 min at 121° C. in order to ensure sterility. The water was removed the next morning in a clean bench and replaced with sterile fermentation medium (autoclaved: 2.2 g/L (NH₄)₂SO₄, 0.02 g/L NaCl, 0.4 g/L MgSO₄×7H₂O, 0.04 g/L CaCl₂×2H₂O, sterilized separately: 2 g/L KH₂PO₄, 15 g/L glucose, 10 mL/L trace element solution M12 [sterile-filtered: 0.2 g/L ZnSO₄×7H₂O, 0.1 g/L MnCl₂×4H₂O, 1.5 g/L Na₃ citrate×2H₂O, 0.1 g/L CuSO₄×5H₂O, 0.002 g/L NiCl₂×6H₂O, 0.003 g/L Na₂MoO₄×2H₂O, 0.03 g/L H₃BO₃, 1 g/L FeSO₄×7H₂O]). Subsequently, the pO₂probes were calibrated by means of a one-point calibration (stirrer: 600 rpm/aeration: 10 sL/h air), and the feed, correcting agent and induction agent lines cleaned by means of cleaning-in-place. To this end, the hoses were flushed with 70% ethanol, then with 1 M NaOH, then with sterile demineralized water and finally filled with the particular media.

Using 100 μL from a cryoculture, the strain (P. putida KT2440 Δupp+pACYCATh5-{PrhaSR}[rhaSR_Ec]{PrhaBAD}[rhlABC_Pa]{Talk}[araC_Ec]{ParaBAD}[rmlBDAC_Pa]{Tal k} was first grown overnight at 30° C. and 200 rpm for approximately 18 h in 25 mL of LB1 medium (10 g/L casein hydrolysate, 5 g/L yeast extract, 1 g/L NaCl) in a 250 mL baffled flask containing 50 mg/L kanamycin. After measurement of the optical density of the culture, 50 mL of sterile seed medium (autoclaved: 4.4 g/L Na₂HPO₄*2H₂O, 1.5 g/L KH₂PO₄, 1 g/L NH₄Cl, 10 g/L yeast extract, sterilized separately: 20 g/L glucose, 0.2 g/L MgSO₄*7H₂O, 0.006 g/L FeCl₃, 0.015 g/L CaCl₂, 1 mL/L trace element solution SL6 [sterile-filtered: 0.3 g/L H₃BO₃, 0.2 g/L CoCl₂×6H₂O, 0.1 g/L ZnSO₄×7H₂O, 0.03 g/L MnCl₂×4H₂O, 0.01 g/L CuCl₂×2H₂O, 0.03 g/L Na₂MoO₄×2H₂O, 0.02 g/L NiCl₂×6H₂O]) in a 500 mL baffled flask were inoculated from the LB preculture using a start OD₆₀₀ of 0.2 and incubated for approximately 7 h at 30° C. and 200 rpm. At an optical density of approximately OD₆₀₀ 8, the main culture was inoculated using a start OD₆₀₀ of 0.7.

In order to inoculate the reactors using an optical density of 0.7, approximately 26 mL were filled in a 30 mL syringe and the reactors were inoculated by means of a needle across a septum.

The following standard program was used:

| | |
|---|---|
| DO regulator | pH regulator |

| Preset | 0% | Preset | 0 | ml/h |
|---|---|---|---|---|
| P | 0.1 | P | 5 | |
| Ti | 300 s | Ti | 200 | s |
| Min | 0% | Min | 0 | mlL/h |
| Max | 100% | Max | 40 | mL/h |

| | | | XO2 (gas | | |
|---|---|---|---|---|---|
| N (Rotation) | from | to | mixture) | from | to |
| Growth and | 0% | 40% | Growth and | 0% | 100% |
| biotransformation | 500 | 1500 | biotransformation | 21% | 21% |
| | rpm | rpm | | | |

| F (gas flow rate) | from | to |
|---|---|---|
| Growth and biotransformation | 35% | 100% |
| | 9 sL/h | 72 sL/h |

| Script | |
|---|---|
| Trigger | 31% DO (1/60 h) |
| activated | |
| Induction, | 3 h after feed |
| rhamnose, | start |
| arabinose | |
| Feed trigger | 50% DO |
| Feed rate | 1.5 [mL/h] |

pH was one-sidedly adjusted to pH 7.0 using ammonia (12.5%). During cultivation and biotransformation, the dissolved oxygen in the culture was kept constant at 30% via stirrer speed and aeration rate. The fermentation was carried out as a fed batch, where, from the feed start, the feeding with 2.5 g/Lh glucose by means of a 500 g/L glucose feed was triggered via a DO peak. The expression of the recombinantly introduced genes was induced 3 h after the feed start by the automatic addition of 0.2% (w/v) rhamnose and 0.2% (w/v) arabinose. The required amounts of induction sugar are based on the fermentation starting volume. For both sugars, 220 g/L stock solutions were used. The production of surfactant started from the time of induction. All online measurement data such as pH, DO, CTR, OTR, but also the flow rates and amount of the substrates such as ammonia solution for pH adjustment, the glucose feed or the inducer flow rates, were logged by the DASGIP fermentation system.

For fermentation analysis, a 10 mL syringe was used to draw and discard 2 mL as forerun from each vessel. This was followed once more by 6 mL being removed from the reactor for the actual analysis. Rhamnolipid content was determined. The fermentation was ended after 65 h.

Rhamnolipid concentration was determined by means of HPLC. 100 μL of the fermentation sample were admixed with 900 μL of 70% (v/v) n-propanol in an Eppendorf tube and shaken at 30 Hz for 1 min in a Retsch mill. Thereafter, the sample was centrifuged at 13 000 rpm for 5 min and the supernatant transferred to a fresh Eppendorf tube. In the event of a further dilution being necessary, this was done using 55% n-propanol. All tubes were closed quickly in order to avoid evaporation. The samples were then transferred to HPLC vials and stored at −20° C. until measurement.

1 ml of acetone was charged in a 2 ml reaction tube using a positive displacement pipette (Combitip) and the reaction tube immediately closed to minimize evaporation. This was followed by the addition of 1 ml of culture broth. After vortexing of the culture broth/acetone mixture, said mixture was centrifuged for 3 min at 13 000 rpm, and 800 μl of the supernatant transferred to an HPLC vial.

An evaporative light scattering detector (Sedex LT-ELSD Model 85LT) was used for detection and quantification of rhamnolipids. The actual measurement was carried out using an Agilent Technologies 1200 Series (Santa Clara, Calif.) and a Zorbax SB-C8 Rapid Resolution column (4.6×150 mm, 3.5 μm, Agilent). The injection volume was 5 μl and the method run time was 20 min. Aqueous 0.1% TFA (trifluoroacetic acid, solution A) and methanol (solution B) was used as mobile phase. The column temperature was 40° C. The ELSD (detector temperature 60° C.) and the DAD (diode array, 210 nm) served as detectors. The gradient used in the method was:

| | Solution B % | Flow rate |
|---|---|---|
| t [min] | by volume | [ml/min] |
| 0.00 | 70% | 1.00 |
| 15.00 | 100% | 1.00 |
| 15.01 | 70% | 1.00 |
| 20.00 | 70% | 1.00 |

3 experiments were carried out, each in parallel to Example 2.

Determined total RL concentration after 65 h: 34 g/L.

Calculated space-time yield: 0.53 g/L*h

### Example 2 (Inventive)

Use was made of the strain P. putida KT2440 Δupp Δ[PP_1016-1018]::galP_Ec +pACYCATh5-{PrhaSR}[rhaSR_Ec]{PrhaBAD}[rhlABC_Pa]{Talk}[araC_Ec]{ParaBAD}[rmlBDAC_Pa]{Tal k})

Construction of a vector for the integration of the galP gene in Pseudomonas putida KT2440 Δupp

A vector for the integration of the galP gene from E. coli K12, encoding a galactose-H⁺ symporter GalP, is prepared by PCR amplification of the gene. The template used is genomic DNA of E. coli K12 W3110. It is intended that the galP gene replace, in P. putida KT2440 Δupp, the genes PP_1016-PP_1018, encoding an ABC transporter permease, an ABC transporter binding protein and an ABC transporter ATP-binding protein. To this end, approximately 680 bp upstream and downstream of the genes PP_1016-PP_1018 are amplified by means of PCR.

The following primers were used for the amplification of the galP gene:

PCR 1: galP
O.BF_FM_1*07
(SEQ ID No 7)
5′-CCAATACATGCCTGACGCTAAAAAACA-3′
O.BF_FM_1*10
(SEQ ID No 8)
5′-TAGACGAGTTAATCGTGAGCGCCTATTT-3′

The following primers were used for the amplification of the homologous regions upstream and downstream of the PP_1016-PP_1018 genes:

PCR 2: Region upstream of PP_1016
O.BF_FM_1*03
(SEQ ID No 9)
5′-GCCGCTTTGGTCCCGGCCGCCAAGGTCATTAAC-3′
O.BF_FM_1*08
(SEQ ID No 10)
5′-TCAGGCATGTATTGGATCCCGAGGTAGT-3′
PCR 3: Region downstream of PP_1018
O.BF_FM_1*02
(SEQ ID No 11)
5′-GCTTGCATGCCTGCAGGCGTGATGTTGTACTTC-3′
O.BF_FM_1*09
(SEQ ID No 12)
5′-CGATTAACTCGTCTACACCATCAATAA-3′

The following parameters were used for the PCR:

| | | | | |
|---|---|---|---|---|
| Denaturation: | 98° C. | 30 | s | |
| Denaturation: | 98° C. | 10 | s | 30x |
| Annealing: | 62° C. | 12 | s | 30x |
| Elongation: | 72° C. | 22 | s | 30x |
| Final elongation: | 72° C. | 5 | min | |

For the amplification, the Phusion™ High-Fidelity Master Mix from NEB (Frankfurt am Main, Germany) was used according to the manufacturer's recommendations. 50 μl of each of the PCR reactions were then resolved on a 1% TAE agarose gel. The PCR, the agarose gel electrophoresis, ethidium bromide staining of the DNA and determination of the PCR fragment sizes were performed in a manner known to a person skilled in the art. PCR fragments of the expected size (PCR 1 (1410 bp, SEQ ID No 13); PCR 2, 675 bp (SEQ ID No 14); PCR 3, 697 bp (SEQ ID No 15)) were amplified. The PCR products were purified using the “QIAquick PCR Purification Kit” from Qiagen as specified by the manufacturer. Using the NEBuilder HiFi DNA Assembly Cloning Kit in accordance with the manufacturer's instructions (NEB; Frankfurt am Main, Germany), the purified PCR products were cloned into a BamHI- and SbfI-cut pKOPp vector (SEQ ID No. 16). Chemically competent E. coli 10 beta cells (NEB, Frankfurt am Main, Germany) were transformed in a manner known to a person skilled in the art. The correct insertion of the target genes was checked by restriction analysis and the authenticity of the introduced homologous regions confirmed by DNA sequencing. The resultant knock-out vector was referred to as pKO_PP_1016-PP_1018::galP (SEQ ID No. 17).

Construction of the strain P. putida KT2440 Δupp Δ[PP_1016-1018]::galP_Ec

The construction of the strain P. putida KT2440 Δupp Δ[PP_1016-1018]::galP_Ec was carried out with the aid of the plasmid pKO_PP_1016-PP_1018::galP and a method described in Graf et al., 2011 (Graf N, Altenbuchner J, Appl. Environ. Micorbiol., 2011, 77(15):5549; DOI: 10.1128/AEM.05055-11). The DNA sequence after replacement of the genes PP_1016-PP_1018 with galP is described in SEQ ID No. 18. The transformation of P. putida KT2440 Δupp Δ[PP_1016-1018]::galP_Ec with the vector pACYCATh5-{PrhaSR}[rhaSR_Ec]{PrhaBAD}[rhlABC_Pa]{Talk}[araC_Ec]{ParaBAD}[rmlBDAC_Pa]{Tal k} was carried out as described in Iwasaki et al. (Iwasaki K, Uchiyama H, Yagi O, Kurabayashi, T, Ishizuka K, Takamura Y, Biosci. Biotech. Biochem. 1994. 58(5):851-854). Thereafter, the cells were plated out on LB agar plates supplemented with kanamycin (50 μg/ml). The plasmid pACYCATh5-{PrhaSR}[rhaSR_Ec]{PrhaBAD}[rhlABC_Pa]{Talk}[araC_Ec]{ParaBAD}[rmlBDAC_Pa]{Talk} (SEQ ID No. 6) has already been described in Example 1. The plasmid DNA from each of 10 clones was isolated and analysed by means of restriction analysis. A strain bearing the plasmid was called P. putida KT2440 Δupp Δ[PP_1016-1018]::galP_Ec pACYCATh5-{PrhaSR}[rhaSR_Ec]{PrhaBAD}[rhlABC_Pa]{Talk}[araC_Ec]{ParaBAD}[rmlBDAC_Pa]{Talk}.

Technical realization was carried out as described in Example 1.

3 experiments were carried out, each in parallel to Example 1.

Determined total RL concentration after 64 h: 44.5 g/L.

Calculated space-time yield: 0.70 g/L*h

### Example 3 (Inventive)

Use is made of strain P. putida KT2440 Δupp Δ[PP_1016-1018]::glf Zm (co Pp)+pACYCATh5-{PrhaSR}[rhaSR_Ec]{PrhaBAD}[rhlABC_Pa]{Talk}[araC_Ec]{ParaBAD}[rmlBDAC_Pa]{Talk})

Construction of a vector for integrating the glf gene in Pseudomonas putida KT2440 Δupp

A vector for the integration of the glf gene from Zymomonas mobilis, encoding a glucose facilitator Glf, is prepared by PCR amplification of the gene codon-optimized for P. putida KT2440. A synthetic DNA fragment is used as template. It is intended that the glf gene replace, in P. putida KT2440 Δupp, the genes PP_1016-PP_1018, encoding an ABC transporter permease, an ABC transporter binding protein and an ABC transporter ATP-binding protein. To this end, approximately 690 bp upstream and downstream of the genes PP_1016PP_1018 are amplified by means of PCR.

The following primers are used for the amplification of the glf gene:

PCR 4: glf
MW_18_02
(SEQ ID No 19)
5′-TACCTCGGGATCCAATACATGTCCAGCGAGTCGTCCCAG-3′
MW_18_03
(SEQ ID No 20)
5′-TTACTTCTGCGAGCGCCACATC-3′

The following primers are used for the amplification of the homologous regions upstream and downstream of the PP_1016-PP_1018 genes:

PCR 5: Region upstream of PP_1016
O.BF_FM_1*03
(SEQ ID No 9)
5′-GCCGCTTTGGTCCCGGCCGCCAAGGTCATTAAC-3′
MW_18_01
(SEQ ID No 21)
5′-CATGTATTGGATCCCGAGGTAG-3′
PCR 6: Region downstream of PP_1018
MW_18_04
(SEQ ID No 22)
5′-GCTCGCAGAAGTAACAATACCTCGTCTACACCATCAATAAGAAAAA
G-3′
O.BF_FM_1*02
(SEQ ID No 11)
5′-GCTTGCATGCCTGCAGGCGTGATGTTGTACTTC-3′

The following parameters are used for the PCR:

| | | | | |
|---|---|---|---|---|
| Denaturation: | 98° C. | 30 | s | |
| Denaturation: | 98° C. | 10 | s | 30x |
| Annealing: | 62° C. | 12 | s | 30x |
| Elongation: | 72° C. | 22 | s | 30x |
| Final elongation: | 72° C. | 5 | min | |

For the amplification, the Phusion™ High-Fidelity Master Mix from NEB (Frankfurt am Main, Germany) is used according to the manufacturer's recommendations. 50 μl of each of the PCR reactions are then resolved on a 1% TAE agarose gel. The PCR, the agarose gel electrophoresis, ethidium bromide staining of the DNA and determination of the PCR fragment sizes are performed in a manner known to a person skilled in the art. PCR fragments of the expected size (PCR 4 (SEQ ID No 23), 1440 bp; PCR 5 (SEQ ID No 24), 670 bp; PCR 6 (SEQ ID No 25), 710 bp) are amplified. The PCR products are purified using the “QIAquick PCR Purification Kit” from Qiagen as specified by the manufacturer. Using the NEBuilder HiFi DNA Assembly Cloning Kit in accordance with the manufacturer's instructions (NEB; Frankfurt am Main, Germany), the purified PCR products are cloned into a BamHI- and SbfI-cut pKOPp vector (SEQ ID No. 16). Chemically competent E. coli 10 beta cells (NEB, Frankfurt am Main, Germany) are transformed in a manner known to a person skilled in the art. The correct insertion of the target genes is checked by restriction analysis and the authenticity of the introduced homologous regions confirmed by DNA sequencing. The resultant knock-out vector is referred to as pKO_PP_1016-PP_1018::glf_Zm (co_Pp) (SEQ ID No. 26).

Construction of the strain P. putida KT2440 Δupp Δ[PP_1016-1018]::glf_Zm (co_Pp)

The construction of the strain P. putida KT2440 Δupp Δ[PP_1016-1018]::glf_Zm (co_Pp) is carried out with the aid of the plasmid pKO_PP_1016-PP_1018::glf_Zm (co_Pp) and a method described in Graf et al., 2011 (Graf N, Altenbuchner J, Appl. Environ. Micorbiol., 2011, 77(15):5549; DOI: 10.1128/AEM.05055-11). The DNA sequence after replacement of the genes PP_1016-PP_1018 with glf is described in SEQ ID No. 27. The transformation of P. putida KT2440 Δupp Δ[PP_1016-1018]::glf_Zm (co_Pp) with the vector pACYCATh5-{PrhaSR}[rhaSR_Ec]{PrhaBAD}[rhlABC_Pa]{Talk}[araC_Ec]{ParaBAD}[rmlBDAC_Pa]{Tal k} is carried out as described in Iwasaki et al. (Iwasaki K, Uchiyama H, Yagi O, Kurabayashi, T, Ishizuka K, Takamura Y, Biosci. Biotech. Biochem. 1994. 58(5):851-854). Thereafter, the cells are plated out on LB agar plates supplemented with kanamycin (50 μg/ml). The plasmid pACYCATh5-{PrhaSR}[rhaSR_Ec]{PrhaBAD}[rhlABC_Pa]{Talk}[araC_Ec]{ParaBAD}[rmlBDAC_Pa]{Talk} (SEQ ID No. 6) has already been described in Example 1. The plasmid DNA from each of 10 clones is isolated and analysed by means of restriction analysis. A strain bearing the plasmid is called P. putida KT2440 Δupp Δ[PP_1016-1018]::glf_Zm (co_Pp) pACYCATh5-{PrhaSR}[rhaSR_Ec]{PrhaBAD}[rhlABC_Pa]{Talk}[araC_Ec]{ParaBAD}[rmlBDAC_Pa]{Talk}.

Technical realization was carried out as described in Example 1.

3 experiments are carried out, each in parallel to Example 1.

A significantly higher total RL concentration after 64 h and a significantly higher calculated space-time yield is observed compared to Example 1.

### Example 4 (Inventive)

Use is made of the strain P. putida KT2440 Δupp Δ[PP_1016-1018]::[ptsH_Ec ptsI_Ec crr_Ec ptsG_Ec]+pACYCATh5-{PrhaSR}[rhaSR_Ec]{PrhaBAD}[rhlABC_Pa]{Talk}[araC_Ec]{ParaBAD}[rmlBDAC_Pa]{Talk})

Construction of a vector for the integration of the pts genes in Pseudomonas putida KT2440 Δupp

A vector for the integration of the pts genes from E. coli K12, encoding a phosphoenolpyruvate phosphotransferase system PEP-PTS, is prepared by PCR amplification of the genes ptsH, ptsI, crr and ptsG. ptsH encodes the phosphocarrier protein HPr, ptsI encodes the PTS enzyme I, crr encodes the enzyme IIAGlc and ptsG encodes the glucose-specific PTS enzyme IIBC. The template used is genomic DNA of E. coli K12 W3110. It is intended that the PTS system replace, in P. putida KT2440 Δupp, the genes PP _1016-PP_1018, encoding an ABC transporter permease, an ABC transporter binding protein and an ABC transporter ATP-binding protein. To this end, approximately 690 bp upstream and downstream of the genes PP_1016-PP_1018 are amplified by means of PCR.

The following primers are used for the amplification of the PTS genes:

PCR 7: Operon ptsH/ptsI/crr
O.BF_FM_1*23
(SEQ ID No 28)
5′-TCCAATACATGTTCCAGCAAGAAGTTACC-3′
O.BF_FM_1*22
(SEQ ID No 29)
5′-CCTGAGTTTACTTCTTGATGCGGATAACC-3′
PCR 8: ptsG
O.BF_FM_1*21
(SEQ ID No 30)
5′-AGAAGTAAACTCAGGAGCACTCTCAATT-3′
O.BF_FM_1*26
(SEQ ID No 31)
5′-AGACGAGTTAGTGGTTACGGATGTACTC-3′

The following primers are used for the amplification of the homologous regions upstream and downstream of the PP_1016-PP_1018 genes:

PCR 9: Region upstream of PP_1016
O.BF_FM_1*03
(SEQ ID No 9)
5′-GCCGCTTTGGTCCCGGCCGCCAAGGTCATTAAC-3′
O.BF_FM_1*24
(SEQ ID No 32)
5′-GGAACATGTATTGGATCCCGAGGTAGTG-3′
PCR 10: Region downstream of PP_1018
O.BF_FM_1*25
(SEQ ID No 33)
5′-ACCACTAACTCGTCTACACCATCAATAAG-3′
O.BF_FM_1*02
(SEQ ID No 11)
5′-GCTTGCATGCCTGCAGGCGTGATGTTGTACTTC-3′

The following parameters are used for the PCR:

| | | | | |
|---|---|---|---|---|
| Denaturation: | 98° C. | 30 | s | |
| Denaturation: | 98° C. | 10 | s | 30x |
| Annealing: | 62° C. | 12 | s | 30x |
| Elongation: | 72° C. | 22 | s | 30x |
| Final elongation: | 72° C. | 5 | min | |

For the amplification, the Phusion™ High-Fidelity Master Mix from NEB (Frankfurt am Main, Germany) is used according to the manufacturer's recommendations. 50 μl of each of the PCR reactions are then resolved on a 1% TAE agarose gel. The PCR, the agarose gel electrophoresis, ethidium bromide staining of the DNA and determination of the PCR fragment sizes are performed in a manner known to a person skilled in the art. PCR fragments of the expected size (PCR 7 (SEQ ID No 34), 2595 bp; PCR 8 (SEQ ID No 35), 1469 bp; PCR 9 (SEQ ID No 36), 674 bp; PCR 10 (SEQ ID No 37), 698 bp) are amplified. The PCR products are purified using the “QIAquick PCR Purification Kit” from Qiagen as specified by the manufacturer. Using the NEBuilder HiFi DNA Assembly Cloning Kit in accordance with the manufacturer's instructions (NEB; Frankfurt am Main, Germany), the purified PCR products are cloned into a BamHI- and SbfI-cut pKOPp vector (SEQ ID No. 16). Chemically competent E. coli 10 beta cells (NEB, Frankfurt am Main, Germany) are transformed in a manner known to a person skilled in the art. The correct insertion of the target genes is checked by restriction analysis and the authenticity of the introduced homologous regions confirmed by DNA sequencing. The resultant knock-out vector is referred to as pKO_PP_1016-PP_1018::ptsH_Ec ptsI_Ec crr_Ec ptsG_Ec) (SEQ ID No 38).

Construction of the strain P. putida KT2440 Δupp Δ[PP_1016-1018]::ptsH_Ec ptsI_Ec crr_Ec ptsG_Ec

The construction of the strain P. putida KT2440 Δupp Δ[PP_1016-1018]::ptsH_Ec ptsI_Ec crr_Ec ptsG_Ec is carried out with the aid of the plasmid pKO_PP_1016-PP_1018::ptsH_Ec ptsI_Ec crr_Ec ptsG_Ec and a method described in Graf et al., 2011 (Graf N, Altenbuchner J, Appl. Environ. Microbiol., 2011, 77(15):5549; DOI: 10.1128/AEM.05055-11). The DNA sequence after replacement of the genes PP_1016-PP_1018 with ptsH_Ec ptsI_Ec crr_Ec ptsG_Ec is described in SEQ ID No 39. The transformation of P. putida KT2440 Δupp A[PP_1016-1018]::ptsH_Ec ptsI_Ec crr_Ec ptsG_Ec with the vector pACYCATh5-{PrhaSR}[rhaSR_Ec]{PrhaBAD}[rhlABC_Pa]{Talk}[araC_Ec]{ParaBAD}[rmlBDAC_Pa]{Talk} is carried out as described in Iwasaki et al. (Iwasaki K, Uchiyama H, Yagi O, Kurabayashi, T, Ishizuka K, Takamura Y, Biosci. Biotech. Biochem. 1994. 58(5):851-854). Thereafter, the cells are plated out on LB agar plates supplemented with kanamycin (50 μg/ml). The plasmid pACYCATh5-{PrhaSR}[rhaSR_Ec]{PrhaBAD}[rhlABC_Pa]{Talk}[araC_Ec]{ParaBAD}[rmlBDAC_Pa]{Talk}(SEQ ID No 6) has already been described in Example 1. The plasmid DNA from each of 10 clones is isolated and analysed by means of restriction analysis. A strain bearing the plasmid is called P. putida KT2440 Δupp Δ[PP_1016-1018]::[ptsH_Ec ptsI_Ec crr_Ec ptsG_Ec]pACYCATh5-{PrhaSR}[rhaSR_Ec]{PrhaBAD}[rhlABC_Pa]{Talk}[araC_Ec]{ParaBAD}[rmlBDAC_Pa]{Talk}.

Technical realization is carried out as described in Example 1.

3 experiments are carried out, each in parallel to Example 1.

A significantly higher total RL concentration after 64 h and a significantly higher calculated space-time yield is observed compared to Example 1.

### Example 5 (Inventive)

Use is made of the strain P. putida KT2440 Δupp Δ[PP_1016-1018]::gluP_Bab+pACYCATh5-{PrhaSR}[rhaSR_Ec]{PrhaBAD}[rhlABC_Pa]{Talk}[araC_Ec]{ParaBAD}[rmlBDAC_Pa]{Talk})

Construction of a vector for the integration of the gluP gene in Pseudomonas putida KT2440 Δupp

A vector for the integration of the gluP gene from Brucella abortus, encoding a glucose/galactose transporter GluP, is prepared by PCR amplification of the gene. The template used is a synthetic DNA fragment. It is intended that the gluP gene replace, in P. putida KT2440 Δupp, the genes PP_1016-PP_1018, encoding an ABC transporter permease, an ABC transporter binding protein and an ABC transporter ATP-binding protein. To this end, approximately 680 bp upstream and downstream of the genes PP_1016-PP_1018 are amplified by means of PCR.

The following primers are used for the amplification of the gluP gene:

PCR 11: gluP
MW_18_05
(SEQ ID No 40)
5′-TACCTCGGGATCCAATACATGGCAACTTCCATCCCAAC-3′
MW_18_06
(SEQ ID No 41)
5′-TCAGCTTTTGCTGCCGATGAG-3′

The following primers are used for the amplification of the homologous regions upstream and downstream of the PP_1016-PP_1018 genes:

PCR 5: Region upstream of PP_1016
O.BF_FM_1*03
(SEQ ID No 9)
5′-GCCGCTTTGGTCCCGGCCGCCAAGGTCATTAAC-3′
MW_18_01
(SEQ ID No 21)
5′-CATGTATTGGATCCCGAGGTAG-3′
PCR 12: Region downstream of PP_1018
MW_18_07
(SEQ ID No 42)
5′-TCATCGGCAGCAAAAGCTGACTCGTCTACACCATCAATAAGAAAAA
G-3′
O.BF_FM_1*02
(SEQ ID No 11)
5′-GCTTGCATGCCTGCAGGCGTGATGTTGTACTTC-3′

The following parameters are used for the PCR:

| | | | | |
|---|---|---|---|---|
| Denaturation: | 98° C. | 30 | s | |
| Denaturation: | 98° C. | 10 | s | 30x |
| Annealing: | 62° C. | 12 | s | 30x |
| Elongation: | 72° C. | 22 | s | 30x |
| Final elongation: | 72° C. | 5 | min | |

For the amplification, the Phusion™ High-Fidelity Master Mix from NEB (Frankfurt am Main, Germany) is used according to the manufacturer's recommendations. 50 μl of each of the PCR reactions are then resolved on a 1% TAE agarose gel. The PCR, the agarose gel electrophoresis, ethidium bromide staining of the DNA and determination of the PCR fragment sizes are performed in a manner known to a person skilled in the art. PCR fragments of the expected size (PCR 11 (SEQ ID No 43), 1257 bp; PCR 5 (SEQ ID No 24), 670 bp; PCR 12 (SEQ ID No 44), 710 bp) are amplified. The PCR products are purified using the “QIAquick PCR Purification Kit” from Qiagen as specified by the manufacturer. Using the NEBuilder HiFi DNA Assembly Cloning Kit in accordance with the manufacturer's instructions (NEB; Frankfurt am Main, Germany), the purified PCR products are cloned into a BamHI- and SbfI-cut pKOPp vector (SEQ ID No. 16). Chemically competent E. coli 10 beta cells (NEB, Frankfurt am Main, Germany) are transformed in a manner known to a person skilled in the art. The correct insertion of the target genes is checked by restriction analysis and the authenticity of the introduced homologous regions confirmed by DNA sequencing. The resultant knock-out vector is referred to as pKO_PP_1016-PP_1018::gluP_Bab (SEQ ID No 45).

Construction of the strain P. putida KT2440 Δupp Δ[PP_1016-1018]::gluP_Bab

The construction of the strain P. putida KT2440 Δupp Δ[PP_1016-1018]::gluP_Bab is carried out with the aid of the plasmid pKO_PP_1016-PP_1018::gluP_Bab and a method described in Graf et al., 2011 (Graf N, Altenbuchner J, Appl. Environ. Microbiol., 2011, 77(15):5549; DOI: 10.1128/AEM.05055-11). The DNA sequence after replacement of the genes PP_1016-PP_1018 with gluP is described in SEQ ID No 46. The transformation of P. putida KT2440 Δupp Δ[PP_1016-1018]::gluP_Bab with the vector pACYCATh5-{PrhaSR}[rhaSR_Ec]{PrhaBAD}[rhlABC_Pa]{Talk}[araC_Ec]{ParaBAD}[rmlBDAC_Pa]{Talk} is carried out as described in Iwasaki et al. (Iwasaki K, Uchiyama H, Yagi O, Kurabayashi, T, Ishizuka K, Takamura Y, Biosci. Biotech. Biochem. 1994. 58(5):851-854). Thereafter, the cells are plated out on LB agar plates supplemented with kanamycin (50 μg/ml). The plasmid pACYCATh5-{PrhaSR}[rhaSR_Ec]{PrhaBAD}[rhlABC_Pa]{Talk}[araC_Ec]{ParaBAD}[rmlBDAC_Pa]{Talk} (SEQ ID No 6) has already been described in Example 1. The plasmid DNA from each of 10 clones is isolated and analysed by means of restriction analysis. A strain bearing the plasmid is called P. putida KT2440 Δupp Δ[PP_1016-1018]::gluP_Bab pACYCATh5-{PrhaSR}[rhaSR_Ec]{PrhaBAD}[rhlABC_Pa]{Talk}[araC_Ec]{ParaBAD}[rmlBDAC_Pa]{Talk}.

Technical realization is carried out as described in Example 1.

3 experiments are carried out, each in parallel to Example 1.

A significantly higher total RL concentration after 64 h and a significantly higher calculated space-time yield is observed compared to Example 1.

### Example 6 (Inventive)

Use is made of the strain P. putida KT2440 Δupp Δ[PP_1016-1018]::iolT1_Cg (co Pp)+pACYCATh5-{PrhaSR}[rhaSR_Ec]{PrhaBAD}[rhlABC_Pa]{Talk}[araC_Ec]{ParaBAD}[rmlBDAC_Pa]{Talk})

Construction of a vector for the integration of the iolT1 gene in Pseudomonas putida KT2440 Δupp

A vector for the integration of the iolT 1 gene from Corynebacterium glutamicum ATCC 13032, encoding a myoinositol facilitator IolT1, is prepared by PCR amplification of the gene codon-optimized for P. putida KT2440. A synthetic DNA fragment is used as template. It is intended that the iolT1 gene replace, in P. putida KT2440 Δupp, the genes PP_1016-PP_1018, encoding an ABC transporter permease, an ABC transporter binding protein and an ABC transporter ATP-binding protein. To this end, approximately 680 bp upstream and downstream of the genes PP_1016-PP_1018 are amplified by means of PCR.

The following primers are used for the amplification of the iolT1 gene:

PCR 13: iolT1
MW_18_08
(SEQ ID No 47)
5′-TACCTCGGGATCCAATACATGGCAAGCACCTTTATCCAGGCCGACA
G-3′
MW_18_09
(SEQ ID No 48)
5′-TCAATGGACCTTGCCCTTGCGAATG-3′

The following primers are used for the amplification of the homologous regions upstream and downstream of the PP_1016-PP_1018 genes:

PCR 5: Region upstream of PP_1016
O.BF_FM_1*03
(SEQ ID No 9)
5′-GCCGCTTTGGTCCCGGCCGCCAAGGTCATTAAC-3′
MW_18_01
(SEQ ID No 21)
5′-CATGTATTGGATCCCGAGGTAG-3′
PCR 14: Region downstream of PP_1018
MW_18_10
(SEQ ID No 49)
5′-GCAAGGGCAAGGTCCATTGACTCGTCTACACCATCAATAAGAAAAA
G-3′
O.BF_FM_1*02
(SEQ ID No 11)
5′-GCTTGCATGCCTGCAGGCGTGATGTTGTACTTC-3′

The following parameters are used for the PCR:

| | | | | |
|---|---|---|---|---|
| Denaturation: | 98° C. | 30 | s | |
| Denaturation: | 98° C. | 10 | s | 30x |
| Annealing: | 62° C. | 12 | s | 30x |
| Elongation: | 72° C. | 22 | s | 30x |
| Final elongation: | 72° C. | 5 | min | |

For the amplification, the Phusion™ High-Fidelity Master Mix from NEB (Frankfurt am Main, Germany) is used according to the manufacturer's recommendations. 50 μl of each of the PCR reactions are then resolved on a 1% TAE agarose gel. The PCR, the agarose gel electrophoresis, ethidium bromide staining of the DNA and determination of the PCR fragment sizes are performed in a manner known to a person skilled in the art. PCR fragments of the expected size (PCR 13 (SEQ ID No 50), 1494 bp; PCR 5 (SEQ ID No 24), 670 bp; PCR 14 (SEQ ID No 51), 710 bp) are amplified. The PCR products are purified using the “QIAquick PCR Purification Kit” from Qiagen as specified by the manufacturer. Using the NEBuilder HiFi DNA Assembly Cloning Kit in accordance with the manufacturer's instructions (NEB; Frankfurt am Main, Germany), the purified PCR products are cloned into a BamHI- and SbfI-cut pKOPp vector (SEQ ID No. 16). Chemically competent E. coli 10 beta cells (NEB, Frankfurt am Main, Germany) are transformed in a manner known to a person skilled in the art. The correct insertion of the target genes is checked by restriction analysis and the authenticity of the introduced homologous regions confirmed by DNA sequencing. The resultant knock-out vector is referred to as pKO_PP_1016-PP_1018::iolT1_Cg (co_Pp) (SEQ ID No 52).

Construction of the strain P. putida KT2440 Δupp Δ[PP_1016-1018]::iolT1_Cg (co_Pp)

The construction of the strain P. putida KT2440 Δupp Δ[PP_1016-1018]::iolT1_Cg (co_Pp) is carried out with the aid of the plasmid pKO_PP_1016-PP_1018::iolT1_Cg (co_Pp) and a method described in Graf et al., 2011 (Graf N, Altenbuchner J, Appl. Environ. Microbiol., 2011, 77(15):5549; DOI: 10.1128/AEM.05055-11). The DNA sequence after replacement of the genes PP_1016-PP_1018 with iolT1_Cg (co_Pp) is described in SEQ ID No 53. The transformation of P. putida KT2440 Δupp Δ[PP_1016-1018]::iolT1_Cg (co_Pp) with the vector pACYCATh5-{PrhaSR}[rhaSR_Ec]{PrhaBAD}[rhlABC_Pa]{Talk}[araC_Ec]{ParaBAD}[rmlBDAC_Pa]{Talk} is carried out as described in Iwasaki et al. (Iwasaki K, Uchiyama H, Yagi O, Kurabayashi, T, Ishizuka K, Takamura Y, Biosci. Biotech. Biochem. 1994. 58(5):851-854). Thereafter, the cells are plated out on LB agar plates supplemented with kanamycin (50 μg/ml). The plasmid pACYCATh5-{PrhaSR}[rhaSR_Ec]{PrhaBAD}[rhlABC_Pa]{Talk}[araC_Ec]{ParaBAD}[rmlBDAC_Pa]{Talk}(SEQ ID No 6) has already been described in Example 1. The plasmid DNA from each of 10 clones is isolated and analysed by means of restriction analysis. A strain bearing the plasmid is called P. putida KT2440 Δupp Δ[PP_1016-1018]::iolT1_Cg (co_Pp) pACYCATh5-{PrhaSR}[rhaSR_Ec]{PrhaBAD}[rhlABC_Pa]{Talk}[araC_Ec]{ParaBAD}[rmlBDAC_Pa]{Talk}.

Technical realization is carried out as described in Example 1.

3 experiments are carried out, each in parallel to Example 1.

A significantly higher total RL concentration after 64 h and a significantly higher calculated space-time yield is observed compared to Example 1.

### Example 7 (Inventive)

Use is made of the strain P. putida KT2440 Δupp Δ[PP_1016-1018]::glcP_Ms+pACYCATh5-{PrhaSR}[rhaSR_Ec]{PrhaBAD}[rhlABC_Pa]{Talk}[araC_Ec]{ParaBAD}[rmlBDAC_Pa]{Talk})

Construction of a vector for the integration of the glcP gene in Pseudomonas putida KT2440 Δupp

A vector for the integration of the glcP gene from Mycobacterium smegmatis, encoding an arabinose-proton symporter GlcP, is prepared by PCR amplification of the gene. The template used is a synthetic DNA fragment. It is intended that the glcP gene replace, in P. putida KT2440 Δupp, the genes PP_1016-PP_1018, encoding an ABC transporter permease, an ABC transporter binding protein and an ABC transporter ATP-binding protein. To this end, approximately 680 bp upstream and downstream of the genes PP_1016-PP_1018 are amplified by means of PCR.

The following primers are used for the amplification of the glcP gene:

PCR 15: glcP
MW_18_11
(SEQ ID No 54)
5′-ACTACCTCGGGATCCAATACATGAATGTGATCGGTATCACTCTC-3′
MW_18_12
(SEQ ID No 55)
5′-TCAGTGCCCCAGCGCTTCGG-3′

The following primers are used for the amplification of the homologous regions upstream and downstream of the PP_1016-PP_1018 genes:

PCR 5: Region upstream of PP_1016
O.BF_FM_1*03
(SEQ ID No 9)
5′-GCCGCTTTGGTCCCGGCCGCCAAGGTCATTAAC-3′
MW_18_01
(SEQ ID No 21)
5′-CATGTATTGGATCCCGAGGTAG-3′
PCR 16: Region downstream of PP_1018
MW_18_13
(SEQ ID No 56)
5′-CCGAAGCGCTGGGGCACTGACTCGTCTACACCATCAATAAGAAAAA
G-3′
O.BF_FM_1*02
(SEQ ID No 11)
5′-GCTTGCATGCCTGCAGGCGTGATGTTGTACTTC-3′

The following parameters are used for the PCR:

| | | | | |
|---|---|---|---|---|
| Denaturation: | 98° C. | 30 | s | |
| Denaturation: | 98° C. | 10 | s | 30x |
| Annealing: | 62° C. | 12 | s | 30x |
| Elongation: | 72° C. | 22 | s | 30x |
| Final elongation: | 72° C. | 5 | min | |

For the amplification, the Phusion™ High-Fidelity Master Mix from NEB (Frankfurt am Main, Germany) is used according to the manufacturer's recommendations. 50 μl of each of the PCR reactions are then resolved on a 1% TAE agarose gel. The PCR, the agarose gel electrophoresis, ethidium bromide staining of the DNA and determination of the PCR fragment sizes are performed in a manner known to a person skilled in the art. PCR fragments of the expected size (PCR 15 (SEQ ID No 57), 1517 bp; PCR 5 (SEQ ID No 24), 670 bp; PCR 16 (SEQ ID No 58), 710 bp) are amplified. The PCR products are purified using the “QIAquick PCR Purification Kit” from Qiagen as specified by the manufacturer. Using the NEBuilder HiFi DNA Assembly Cloning Kit in accordance with the manufacturer's instructions (NEB; Frankfurt am Main, Germany), the purified PCR products are cloned into a BamHI- and SbfI-cut pKOPp vector (SEQ ID No. 16). Chemically competent E. coli 10 beta cells (NEB, Frankfurt am Main, Germany) are transformed in a manner known to a person skilled in the art. The correct insertion of the target genes is checked by restriction analysis and the authenticity of the introduced homologous regions confirmed by DNA sequencing. The resultant knock-out vector is referred to as pKO_PP_1016-PP_1018::glcP_Ms (SEQ ID No 59).

Construction of the strain P. putida KT2440 Δupp Δ[PP_1016-1018]::glcP_Ms

The construction of the strain P. putida KT2440 Δupp Δ[PP_1016-1018]::glcP_Ms is carried out with the aid of the plasmid pKO_PP_1016-PP_1018::glcP_Ms and a method described in Graf et al., 2011 (Graf N, Altenbuchner J, Appl. Environ. Microbiol., 2011, 77(15):5549; DOI: 10.1128/AEM.05055-11). The DNA sequence after replacement of the genes PP_1016-PP_1018 with glcP is described in SEQ ID No 60. The transformation of P. putida KT2440 Δupp Δ[PP_1016-1018]::glcP_Ms with the vector pACYCATh5-{PrhaSR}[rhaSR_Ec]{PrhaBAD}[rhlABC_Pa]{Talk}[araC_Ec]{ParaBAD}[rmlBDAC_Pa]{Talk} is carried out as described in Iwasaki et al. (Iwasaki K, Uchiyama H, Yagi O, Kurabayashi, T, Ishizuka K, Takamura Y, Biosci. Biotech. Biochem. 1994. 58(5):851-854). Thereafter, the cells are plated out on LB agar plates supplemented with kanamycin (50 μg/ml). The plasmid pACYCATh5-{PrhaSR}[rhaSR_Ec]{PrhaBAD}[rhlABC_Pa]{Talk}[araC_Ec]{ParaBAD}[rmlBDAC_Pa]{Talk}(SEQ ID No 6) has already been described in Example 1. The plasmid DNA from each of 10 clones is isolated and analysed by means of restriction analysis. A strain bearing the plasmid is called P. putida KT2440 Δupp Δ[PP_1016-1018]::glcP_Ms pACYCATh5-{PrhaSR}[rhaSR_Ec]{PrhaBAD}[rhlABC_Pa]{Talk}[araC_Ec]{ParaBAD}[rmlBDAC_Pa]{Talk}.

Technical realization is carried out as described in Example 1.

3 experiments are carried out, each in parallel to Example 1.

A significantly higher total RL concentration after 64 h and a significantly higher calculated space-time yield is observed compared to Example 1.

### Example 8 (Inventive)

Use is made of the strain P. putida KT2440 Δupp Δ[PP_1016-1018]::glcU_Bs (co_Pp)+pACYCATh5-{PrhaSR}[rhaSR_Ec]{PrhaBAD}[rhlABC_Pa]{Talk}[araC_Ec]{ParaBAD}[rmlBDAC_Pa]{Talk})

Construction of a vector for the integration of the glcU gene in Pseudomonas putida KT2440 Δupp

A vector for the integration of the glcU gene from Bacillus subtilis, encoding a glucose uptake protein GlcU, is prepared by PCR amplification of the gene codon-optimized for P. putida KT2440. The template used is a synthetic DNA fragment. It is intended that the glcU gene replace, in P. putida KT2440 Δupp, the genes PP_1016-PP_1018, encoding an ABC transporter permease, an ABC transporter binding protein and an ABC transporter ATP-binding protein. To this end, approximately 680 bp upstream and downstream of the genes PP_1016-PP_1018 are amplified by means of PCR.

The following primers are used for amplification of the glcU gene:

PCR 17: glcU
MW_18_14
(SEQ ID No 61)
5′-TACCTCGGGATCCAATACATGGACTTGTTGCTGGCTCTG-3′
MW_18_15
(SEQ ID No 62)
5′-CTAGCTGTTGGTCTTGGCGATG-3′

The following primers are used for the amplification of the homologous regions upstream and downstream of the PP_1016-PP_1018 genes:

PCR 5: Region upstream of PP_1016
O.BF_FM_1*03
(SEQ ID No 9)
5′-GCCGCTTTGGTCCCGGCCGCCAAGGTCATTAAC-3′
MW_18_01
(SEQ ID No 21)
5′-CATGTATTGGATCCCGAGGTAG-3′
PCR 18: Region downstream of PP_1018
MW_18_16
(SEQ ID No 63)
5′-TCGCCAAGACCAACAGCTAGCTCGTCTACACCATCAATAAGAAAAA
G-3′
O.BF_FM_1*02
(SEQ ID No 11)
5′-GCTTGCATGCCTGCAGGCGTGATGTTGTACTTC-3′

The following parameters are used for the PCR:

| | | | | |
|---|---|---|---|---|
| Denaturation: | 98° C. | 30 | s | |
| Denaturation: | 98° C. | 10 | s | 30x |
| Annealing: | 62° C. | 12 | s | 30x |
| Elongation: | 72° C. | 22 | s | 30x |
| Final elongation: | 72° C. | 5 | min | |

For the amplification, the Phusion™ High-Fidelity Master Mix from NEB (Frankfurt am Main, Germany) is used according to the manufacturer's recommendations. 50 μl of each of the PCR reactions are then resolved on a 1% TAE agarose gel. The PCR, the agarose gel electrophoresis, ethidium bromide staining of the DNA and determination of the PCR fragment sizes are performed in a manner known to a person skilled in the art. PCR fragments of the expected size (PCR 17 (SEQ ID No 64), 882 bp; PCR 5 (SEQ ID No 24), 670 bp; PCR 18 (SEQ ID No 65), 710 bp) are amplified. The PCR products are purified using the “QIAquick PCR Purification Kit” from Qiagen as specified by the manufacturer. Using the NEBuilder HiFi DNA Assembly Cloning Kit in accordance with the manufacturer's instructions (NEB; Frankfurt am Main, Germany), the purified PCR products are cloned into a BamHI- and SbfI-cut pKOPp vector (SEQ ID No. 16). Chemically competent E. coli 10 beta cells (NEB, Frankfurt am Main, Germany) are transformed in a manner known to a person skilled in the art. The correct insertion of the target genes is checked by restriction analysis and the authenticity of the introduced homologous regions confirmed by DNA sequencing. The resultant knock-out vector is referred to as pKO_PP_1016-PP_1018::glcU_Bs (SEQ ID No 66).

Construction of the starin P. putida KT2440 Δupp Δ[PP_1016-1018]::glcU_Bs

The construction of the strain P. putida KT2440 Δupp Δ[PP_1016-1018]::glcU_Bs is carried out with the aid of the plasmid pKO_PP_1016-PP_1018::glcU_Bs and a method described in Graf et al., 2011 (Graf N, Altenbuchner J, Appl. Environ. Microbiol., 2011, 77(15):5549; DOI: 10.1128/AEM.05055-11). The DNA sequence after replacement of the genes PP_1016-PP_1018 with glcU is described in SEQ ID No 67. The transformation of P. putida KT2440 Δupp Δ[PP_1016-1018]::glcU_Bs with the vector pACYCATh5-{PrhaSR}[rhaSR_Ec]{PrhaBAD}[rhlABC_Pa]{Talk}[araC_Ec]{ParaBAD}[rmlBDAC_Pa]{Talk} is carried out as described in Iwasaki et al. (Iwasaki K, Uchiyama H, Yagi O, Kurabayashi, T, Ishizuka K, Takamura Y, Biosci. Biotech. Biochem. 1994. 58(5):851-854). Thereafter, the cells are plated out on LB agar plates supplemented with kanamycin (50 μg/ml). The plasmid pACYCATh5-{PrhaSR}[rhaSR_Ec]{PrhaBAD}[rhlABC_Pa]{Talk}[araC_Ec]{ParaBAD}[rmlBDAC_Pa]{Talk} (SEQ ID No 6) has already been described in Example 1. The plasmid DNA from each of 10 clones is isolated and analysed by means of restriction analysis. A strain bearing the plasmid is called P. putida KT2440 Δupp Δ[PP_1016-1018]::glcU_Bs pACYCATh5-{PrhaSR}[rhaSR_Ec]{PrhaBAD}[rhlABC_Pa]{Talk}[araC_Ec]{ParaBAD}[rmlBDAC_Pa]{Talk}.

Technical realization is carried out as described in Example 1.

3 experiments are carried out, each in parallel to Example 1.

A significantly higher total RL concentration after 64 h and a significantly higher calculated space-time yield is observed compared to Example 1.

### Example 9 (Inventive)

Use is made of the strain P. putida KT2440 Δupp Δ[PP_1016-1018]:SemiSWEET_Lb (co Pp)+pACYCATh5-{PrhaSR}[rhaSR_Ec]{PrhaBAD}[rhlABC_Pa]{Talk}[araC_Ec]{ParaBAD}[rmlBDAC_Pa]{Talk})

Construction of a vector for the integration of the SemiSWEET gene in Pseudomonas putida KT2440 Δupp

A vector for the integration of the semiSWEET gene from Leptospira biflexa, encoding a sugar transporter semisweet, is prepared by PCR amplification of the gene codon-optimized for P. putida KT2440. The template used is a synthetic DNA fragment. It is intended that the semiSWEET gene replace, in P. putida KT2440 Δupp, the genes PP_1016-PP_1018, encoding an ABC transporter permease, an ABC transporter binding protein and an ABC transporter ATP-binding protein. To this end, approximately 680 bp upstream and downstream of the genes PP_1016-PP_1018 are amplified by means of PCR.

The following primers are used for the amplification of the semiSWEET gene:

PCR 19: semiSWEET
MW_18_17
(SEQ ID No 68)
5′-TACCTCGGGATCCAATACATGGAAAACTTGATCGGCTATGTG-3′
MW_18_18
(SEQ ID No 69)
5′-TCAGGTTTGGTTGCCCTCGGTCAG-3′

The following primers are used for the amplification of the homologous regions upstream and downstream of the PP_1016-PP_1018 genes:

PCR 5: Region upstream of PP_1016
O.BF_FM_1*03
(SEQ ID No 9)
5′-GCCGCTTTGGTCCCGGCCGCCAAGGTCATTAAC-3′
MW_18_01
(SEQ ID No 21)
5′-CATGTATTGGATCCCGAGGTAG-3′
PCR 20: Region downstream of PP_1018
MW_18_19
(SEQ ID No 70)
5′-CCGAGGGCAACCAAACCTGACTCGTCTACACCATCAATAAGAAAAA
G-3′
O.BF_FM_1*02
(SEQ ID No 11)
5′-GCTTGCATGCCTGCAGGCGTGATGTTGTACTTC-3′

The following parameters are used for the PCR:

| | | | | |
|---|---|---|---|---|
| Denaturation: | 98° C. | 30 | s | |
| Denaturation: | 98° C. | 10 | s | 30x |
| Annealing: | 62° C. | 12 | s | 30x |
| Elongation: | 72° C. | 22 | s | 30x |
| Final elongation: | 72° C. | 5 | min | |

For the amplification, the Phusion™ High-Fidelity Master Mix from NEB (Frankfurt am Main, Germany) is used according to the manufacturer's recommendations. 50 μl of each of the PCR reactions are then resolved on a 1% TAE agarose gel. The PCR, the agarose gel electrophoresis, ethidium bromide staining of the DNA and determination of the PCR fragment sizes are performed in a manner known to a person skilled in the art. PCR fragments of the expected size (PCR 19 (SEQ ID No 71), 276 bp; PCR 5 (SEQ ID No 24), 670 bp; PCR 20 (SEQ ID No 72), 710 bp) are amplified. The PCR products are purified using the “QIAquick PCR Purification Kit” from Qiagen as specified by the manufacturer. Using the NEBuilder HiFi DNA Assembly Cloning Kit in accordance with the manufacturer's instructions (NEB; Frankfurt am Main, Germany), the purified PCR products are cloned into a BamHI- and SbfI-cut pKOPp vector (SEQ ID No. 16). Chemically competent E. coli 10 beta cells (NEB, Frankfurt am Main, Germany) are transformed in a manner known to a person skilled in the art. The correct insertion of the target genes is checked by restriction analysis and the authenticity of the introduced homologous regions confirmed by DNA sequencing. The resultant knock-out vector is referred to as pKO_PP_1016-PP_1018::SemiSWEET_Lb (co_Pp) (SEQ ID No 73).

Construction of the strain P. putida KT2440 Δupp Δ[PP_1016-1018]::semiSWEET_Lb (co_Pp)

The construction of the strain P. putida KT2440 Δupp Δ[PP_1016-1018]::semiSWEET_Lb (co_Pp) is carried out with the aid of the plasmid pKO_PP_1016-PP_1018::semiSWEET_Lb (co_Pp) and a method described in Graf et al., 2011 (Graf N, Altenbuchner J, Appl. Environ. Microbiol., 2011, 77(15):5549; DOI: 10.1128/AEM.05055-11). The DNA sequence after replacement of the genes PP_1016-PP_1018 with semiSWEET is described in SEQ ID No 74. The transformation of P. putida KT2440 Δupp APP_1016-10181: semiSWEET_Lb (co_Pp) with the vector pACYCATh5-{PrhaSR}[rhaSR_Ec]{PrhaBAD}[rhlABC_Pa]{Talk}[araC_Ec]{ParaBAD}[rmlBDAC_Pa]{Talk} is carried out as described in Iwasaki et al. (Iwasaki K, Uchiyama H, Yagi O, Kurabayashi, T, Ishizuka K, Takamura Y, Biosci. Biotech. Biochem. 1994. 58(5):851-854). Thereafter, the cells are plated out on LB agar plates supplemented with kanamycin (50 μg/ml). The plasmid pACYCATh5-{PrhaSR}[rhaSR_Ec]{PrhaBAD}[rhlABC_Pa]{Talk}[araC_Ec]{ParaBAD}[rmlBDAC_Pa]{Talk}(SEQ ID No 6) has already been described in Example 1. The plasmid DNA from each of 10 clones is isolated and analysed by means of restriction analysis. A strain bearing the plasmid is called P. putida KT2440 Δupp Δ[PP_1016-1018]::semiSWEET_Lb (co_Pp) pACYCATh5-{PrhaSR}[rhaSR_Ec]{PrhaBAD}[rhlABC_Pa]{Talk}[araC_Ec]{ParaBAD}[rmlBDAC_Pa]{Talk}.

Technical realization is carried out as described in Example 1.

3 experiments are carried out, each in parallel to Example 1.

A significantly higher total RL concentration after 64 h and a significantly higher calculated space-time yield is observed compared to Example 1.

1. A rhamnolipid-making cell, wherein the rhamnolipid-making cell is genetically modified such that the rhamnolipid-making cell has a decreased activity, compared to the wild type thereof, of an ABC glucose transporter and an increased activity, compared to the wild type thereof, of at least one non-ABC glucose transporter.

2. The rhamnolipid-making cell according to claim 1, wherein the non-ABC glucose transporter is selected from the group consisting of
phosphoenolpyruvate phosphotransferase systems of EC 2.7.3.9,
galactose permeases,
glucose facilitators,
myo-inositol transporters,
glucose permeases, and
glucose/galactose transporters.

3. The rhamnolipid-making cell according to claim 1, wherein the non-ABC glucose transporter is selected from the group consisting of enzymes encoded by a galP, glf, iolT1, glcP, gluP, SemiSWEET or glcU gene and PTS systems consisting of the components enzyme I, HPr, enzyme IIA, enzyme IIB and enzyme IIC, it being possible for enzymes IIA, IIB and IIC to be present as fusion proteins, or enzymes having a polypeptide sequence in which up to 25% of the amino acid residues of the galP, glf, iolT1, glcP, gluP, SemiSWEET or glcU gene-encoded enzymes and PTS systems are modified by deletion, insertion, substitution or a combination thereof and which still has at least 10% of the enzymatic activity of the galP, glf, iolT1, glcP, gluP, SemiSWEET or glcU gene-encoded enzyme and PTS system.

4. The rhamnolipid-making cell according to claim 1, wherein the rhamnolipid-making cell is selected from the group consisting of Burkholderia sp., Burkholderia thailandensis, Pseudomonas sp., Pseudomonas putida, Pseudomonas aeruginosa, Pseudomonas oleovorans, Pseudomonas stutzeri, Pseudomonas chlororaphis, Pseudomonas fluorescens, Pseudomonas citronellolis, Pseudomonas resinovorans, Comamonas testosterone, Aeromonas hydrophila, Cupriavidus necator, Alcaligenes latus and Ralstonia eutropha.

5. The rhamnolipid-making cell according to claim 1, wherein the rhamnolipid-making cell has been genetically modified such that the rhamnolipid-making cell, compared to the wild type thereof, has an increased activity of at least one of the enzymes selected from the group E₁, E₂ and E₃,
the enzyme E₁ being able to catalyse the conversion of 3-hydroxyalkanoyl-ACP via 3-hydroxyalkanoyl-3-hydroxyalkanoic acid-ACP to hydroxyalkanoyl-3-hydroxyalkanoic acid,
the enzyme E₂ being a rhamnosyltransferase I and being able to catalyse the conversion of dTDP-rhamnose and 3-hydroxyalkanoyl-3-hydroxyalkanoate to α-L-rhamnopyranosyl-3-hydroxyalkanoyl-3-hydroxyalkanoate, and
the enzyme E₃ being a rhamnosyltransferase II and being able to catalyse the conversion of dTDP-rhamnose and α-L-rhamnopyranosyl-3-hydroxyalkanoyl-3-hydroxyalkanoate to α-L-rhamnopyranosyl-(1-2)-α-L-rhamnopyranosyl-3-hydroxyalkanoyl-3-hydroxyalkanoate.

6. The rhamnolipid-making cell according to claim 5, wherein E₁, E₂ and E₃ are encoded by an rhlA gene, an rhlB gene and an rhlC gene, respectively, or are enzymes having a polypeptide sequence in which up to 25% of the amino acid residues are modified with respect to the enzymes encoded by an rhl gene by deletion, insertion, substitution or a combination thereof and which still has at least 10% of the enzymatic activity of the enzyme having the reference sequence of the enzymes encoded by an rhl gene.

7. The rhamnolipid-making cell according to at claim 1, wherein the rhamnolipid-making cell has been genetically modified such that the rhamnolipid-making cell, compared to the wild type thereof, has a decreased activity of at least one enzyme E₄, which catalyses the conversion of D-glucose and quinone to D-glucono-1,5-lactone and quinol.

8. The rhamnolipid-making cell according to claim 7, wherein E₄ is a glucose 1-dehydrogenase of EC 1.1.5.2.

9. The method for producing rhamnolipids, comprising the method steps of
I) contacting a rhamnolipid-making cell according to claim 1 with a medium containing a carbon source
II) culturing the rhamnolipid-making cell under conditions allowing the rhamnolipid-making cell to make rhamnolipid from the carbon source and
III) optionally isolating the rhamnolipids made.

10. A method for making a product selected from the group consisting of cosmetic formulation, dermatological formulation, pharmaceutical formulation, crop-protection formulation, care product, cleaning agent, and surfactant concentrate, the method comprising the method according to claim 9.

11. The rhamnolipid-making cell according to claim 2, wherein the non-ABC glucose transporter is selected from the group consisting of enzymes encoded by a galP, glf, iolT1, glcP, gluP, SemiSWEET or glcU gene and PTS systems consisting of the components enzyme I, HPr, enzyme IIA, enzyme JIB and enzyme IIC, it being possible for enzymes IIA, IIB and IIC to be present as fusion proteins, or enzymes having a polypeptide sequence in which up to 25% of the amino acid residues of the galP, glf, iolT1, glcP, gluP, SemiSWEET or glcU gene-encoded enzymes and PTS systems are modified by deletion, insertion, substitution or a combination thereof and which still has at least 10% of the enzymatic activity of the galP, glf, iolT1, glcP, gluP, SemiSWEET or glcU gene-encoded enzyme and PTS system.

12. The rhamnolipid-making cell according to claim 2, wherein the rhamnolipid-making cell is selected from the group consisting of Burkholderia sp., Burkholderia thailandensis, Pseudomonas sp., Pseudomonas putida, Pseudomonas aeruginosa, Pseudomonas oleovorans, Pseudomonas stutzeri, Pseudomonas chlororaphis, Pseudomonas fluorescens, Pseudomonas citronellolis, Pseudomonas resinovorans, Comamonas testosteroni, Aeromonas hydrophila, Cupriavidus necator, Alcaligenes latus and Ralstonia eutropha.

13. The rhamnolipid-making cell according to claim 3, wherein the rhamnolipid-making cell is selected from the group consisting of Burkholderia sp., Burkholderia thailandensis, Pseudomonas sp., Pseudomonas putida, Pseudomonas aeruginosa, Pseudomonas oleovorans, Pseudomonas stutzeri, Pseudomonas chlororaphis, Pseudomonas fluorescens, Pseudomonas citronellolis, Pseudomonas resinovorans, Comamonas testosteroni, Aeromonas hydrophila, Cupriavidus necator, Alcaligenes latus and Ralstonia eutropha.

14. The rhamnolipid-making cell according to claim 2, wherein the rhamnolipid-making cell has been genetically modified such that the rhamnolipid-making cell, compared to the wild type thereof, has an increased activity of at least one of the enzymes selected from the group E₁, E₂ and E₃,
the enzyme E₁ being able to catalyse the conversion of 3-hydroxyalkanoyl-ACP via 3-hydroxyalkanoyl-3-hydroxyalkanoic acid-ACP to hydroxyalkanoyl-3-hydroxyalkanoic acid,
the enzyme E₂ being a rhamnosyltransferase I and being able to catalyse the conversion of dTDP-rhamnose and 3-hydroxyalkanoyl-3-hydroxyalkanoate to α-L-rhamnopyranosyl-3-hydroxyalkanoyl-3-hydroxyalkanoate, and
the enzyme E₃ being a rhamnosyltransferase II and being able to catalyse the conversion of dTDP-rhamnose and α-L-rhamnopyranosyl-3-hydroxyalkanoyl-3-hydroxyalkanoate to α-L-rhamnopyranosyl-(1-2)-α-L-rhamnopyranosyl-3-hydroxyalkanoyl-3-hydroxyalkanoate.

15. The rhamnolipid-making cell according to claim 3, wherein the rhamnolipid-making cell has been genetically modified such that the rhamnolipid-making cell, compared to the wild type thereof, has an increased activity of at least one of the enzymes selected from the group E₁, E₂ and E₃,
the enzyme E₁ being able to catalyse the conversion of 3-hydroxyalkanoyl-ACP via 3-hydroxyalkanoyl-3-hydroxyalkanoic acid-ACP to hydroxyalkanoyl-3-hydroxyalkanoic acid,
the enzyme E₂ being a rhamnosyltransferase I and being able to catalyse the conversion of dTDP-rhamnose and 3-hydroxyalkanoyl-3-hydroxyalkanoate to α-L-rhamnopyranosyl-3-hydroxyalkanoyl-3-hydroxyalkanoate, and
the enzyme E₃ being a rhamnosyltransferase II and being able to catalyse the conversion of dTDP-rhamnose and α-L-rhamnopyranosyl-3-hydroxyalkanoyl-3-hydroxyalkanoate to α-L-rhamnopyranosyl-(1-2)-α-L-rhamnopyranosyl-3-hydroxyalkanoyl-3-hydroxyalkanoate.

16. The rhamnolipid-making cell according to claim 14, wherein E₁, E₂ and E₃ are encoded by an rhlA gene, an rhlB gene and an rhlC gene, respectively, or are enzymes having a polypeptide sequence in which up to 25% of the amino acid residues are modified with respect to the enzymes encoded by an rhl gene by deletion, insertion, substitution or a combination thereof and which still has at least 10% of the enzymatic activity of the enzyme having the reference sequence of the enzymes encoded by an rhl gene.

17. The rhamnolipid-making cell according to claim 15, wherein E₁, E₂ and E₃ are encoded by an rhlA gene, an rhlB gene and an rhlC gene, respectively, or are enzymes having a polypeptide sequence in which up to 25% of the amino acid residues are modified with respect to the enzymes encoded by an rhl gene by deletion, insertion, substitution or a combination thereof and which still has at least 10% of the enzymatic activity of the enzyme having the reference sequence of the enzymes encoded by an rhl gene.

18. The rhamnolipid-making cell according to claim 2, wherein the rhamnolipid-making cell has been genetically modified such that it, compared to the wild type thereof, has a decreased activity of at least one enzyme E₄, which catalyses the conversion of D-glucose and quinone to D-glucono-1,5-lactone and quinol.

19. The rhamnolipid-making cell according to claim 3, wherein the rhamnolipid-making cell has been genetically modified such that it, compared to the wild type thereof, has a decreased activity of at least one enzyme E₄, which catalyses the conversion of D-glucose and quinone to D-glucono-1,5-lactone and quinol.

20. The rhamnolipid-making cell according to claim 18, wherein E₄ is a glucose 1-dehydrogenase of EC 1.1.5.2.


SEQUENCE LISTING (74 sequences)

SEQ ID NO: 1 - DNA, 3272 bp, Artificial Sequence
  synthetic operon
ggcccaggaggggggatctggcatttttgggaggtgtgaaatgcggcgcgaaagtctgttggtatcggtttgcaagggcctgcgggtacatgtcgagcgcgttgggcaggatcccgggcgcagcacggtgatgctggtcaacggcgcgatggcgaccaccgcctcgttcgcccggacctgcaagtgcctggccgaacatttcaacgtggtgctgttcgacctgcccttcgccgggcagtcgcgtcagcacaacccgcagcgggggttgatcaccaaggacgacgaggtggaaatcctcctggcgctgatcgagcgcttcgaggtcaatcacctggtctccgcgtcctggggcggtatctccacgctgctggcgctgtcgcgcaatccgcgcggcatccgcagctcggtggtgatggcattcgcccctggactgaaccaggcgatgctcgactacgtcgggcgggcgcaggcgctgatcgagctggacgacaagtcggcgatcggccatctgctcaacgagaccgtcggcaaatacctgccgccgcgcctgaaagccagcaaccatcagcacatggcttcgctggccaccggcgaatacgagcaggcgcgctttcacatcgaccaggtgctggcgctcaacgatcggggctacctggcttgcctggagcggatccagagccacgtgcatttcatcaacggcagctgggacgaatacaccaccgccgaggacgcccgccagttccgcgactacctgccgcactgcagtttctcgcgggtggagggcaccgggcatttcctcgacctggagtccaagctggccgcggtacgcgtgcaccgcgccctgctcgagcacctgctgaagcaaccggagccgcagcgggcggaacgcgcggcgggattccacgagatggccatcggctacgcctgaacccttgacctgcgaagacccggcctggccgggctttgcggttgcataacgcacggagtagcaccatgcacgccatcctcatcgccatcggctcggccggcgacgtatttcccttcatcggcctggcccggaccctgaaattgcgcgggcaccgcgtgagcctctgcaccatcccggtgtttcgcgacgcggtggagcagcacggcatcgcgttcgtcccgctgagcgacgaactgacctaccgccggaccatgggcgatccgcgcctgtgggaccccaagacgtccttcggcgtgctctggcaaaccatcgccgggatgatcgagccggtctacgagtacgtctcggcgcagcgccatgacgacatcgtggtggtcggctcgctctgggcgctgggcgcacgcatcgctcacgagaagtacgggattccctacctgtccgcgcaggtctcgccatcgaccttgttgtcggcgcacctgccgccggtacaccccaagttcaacgtgcccgagcagatgccgctggcgatgcgcaagctgctctggcgctgcatcgagcgcttcaagctggatcgcacctgcgcgccggatatcaacgcggtgcggcgcaaggtcggcctggagacgccggtgaagcgcatcttcacccaatggatgcattcgccgcagggcgtggtctgcctgttcccggcctggttcgcgccgccccagcaggattggccgcaacccctgcacatgaccggcttcccgctgttcgacggcagtatcccggggaccccgctcgacgacgaactgcaacgctttctcgatcagggcagccggccgctggtgttcacccagggctcgaccgaacacctgcagggcgacttctacgccatggccctgcgcgcgctggaacgcctcggcgcgcgtgggatcttcctcaccggcgccggccaggaaccgctgcgcggcttgccgaaccacgtgctgcagcgcgcctacgcgccactgggagccttgctgccatcgtgcgccgggctggtccatccgggcggtatcggcgccatgagcctggccttggcggcgggggtgccgcaggtgctgctgccctgcgcccacgaccagttcgacaatgccgaacggctggtccggctcggctgcgggatgcgcctgggcgtgccattgcgcgagcaggagttgcgcggggcgctgtggcgcttgctcgaggacccggccatggcggcggcctgtcggcgtttcatggaattgtcacaaccgcacagtatcgcttgcggtaaagcggcccaggtggtcgaacgttgtcatagggagggggatgcgcgatggctgaaggctgcgtcctgacctacgggagaagaacgatcatggaccggatagacatgggcgtgctggtggtactgttcaatcctggcgacgacgacctggaacaccttggcgaactggcggcggcgtttccgcaactgcgcttccttgccgtcgacaactcaccgcacagcgatccgcagcgcaatgcccggctgcgcgggcaaggcatcgccgtgctgcaccacggcaaccggcagggcatcgccggcgccttcaaccagggactcgacgcgctattccggcgtggcgtgcagggtgtgctgctgctcgaccaggactcccgtcccggcggcgccttcctcgccgcccagtggcgcaacctgcaggcgcgcaacggtcaggcctgcctgctcggcccacggatcttcgaccggggtgaccggcgcttcctgccggccatccatctcgacggactgacgctcaggcaattgtctctggacggcctgacgaccccgcagcgcacctcgttcctgatctcctccggctgcctgctgacccgcgaggcctaccagcgcctcggccacttcgacgaggaactgttcatcgaccacgtggacaccgaatacagcctgcgcgcccaggcgctggacgtgcccctgtacgtcgacccgcggctggtcctcgagcaccgcatcggcacgcgcaagacccgccgcctcggcggtctcagcctcagcgcgatgaaccacgccccgctgcgccgctactacctggcgcgcaacggcctgctggtcctgcgccgctacgcccggtcctcgccgctggccctgctggcgaacctgccgaccctgacccagggcctcgcggtgctcctgctcgaacgcgacaagctgctcaagctgcgctgcctgggctggggcctgtgggacggcctgcggggacgcggcggcgcgctggagaccaaccgcccgcgcctgctgaagcgcctcgccggcccggccgtggcgtccgtagcttccggcaaggccaaggcctag

SEQ ID NO: 2 - DNA, 3413 bp, Artificial Sequence
  synthetic operon
ttaattaacaggaggaggtatgactcatgacgattctcgtgaccggcagcgccggcttcatcggcgccaatttcgtgctcgactggctggccctgcatgacgagccggtggtcagcctcgacaagctcacctacgccggcaaccggcagaacctcgccagcctcgacggcgacgcccggcacaccttcgtcgccggcgatatcggcgatagccagctggtagcccgcctgctcgccgagcaccagccgcgggcgatcctcaacttcgccgcggaatcccatgtggaccgctcgatccacggccccgaggacttcatccagaccaacatcgtcggcaccttccgcctgctggaagaagtgcgcgcctactggggcgcgctggagccggaagcgaaggcggcattccgcttcctccacgtctccaccgacgaagtctatggctcgctggcaccgagcgatccggccttcaccgagaacaaccgctacgagccgaacagtccctactcggcgtccaaggcggcctccgaccacctagtgcgggcctatcaccacacctatgggctgccggtgctgaccaccaactgctcgaacaactacggcccgtaccacttcccggaaaagctcatcccactggtgatccacaacgccctggccggcaagccgctgccgatctacggcgacggccagcagatccgcgactggctctacgtcaaggaccattgcagcgccatccgccgggtcctcgaagccgggcaactgggcgagacctacaatgtcggcggctggaacgaaaaggccaacctcgacgtggtcgagaccctctgcgccatcctcgaccaggagcagccgcgcgccgacggccgcagctatcgcgagcagatcaccttcgtcaaggatcgtccgggccatgatcgccgctacgccatcgatgccacgcgcctggagcgcgagctgggctggaagccggcggaaaccttcgagaccggcatccgcaagaccgtgcgctggtacctggacaaccaggactgggtggccaacgtaaccagcggtgcctaccgcgagtgggtgggtaagcagtacgcatgaaccggatccttctcctcggcgccaacggccaggtcggctgggagctgcagcgcgccctggcgccgctgggcgaactgctggtctgtgaccgtcggcgcgccgatctcgccgaccccgaaggcctggcgcgactggttcgcgccgagcggccgcagttcatcgtcaacgccggtgcctacaccgcggtggacaaggccgagagcgatgccgacaacgcccgcctgatcaatgcccgcgccgtcgcggtactggccgaggaggccgcggcctgcggcgcctggctggtgcattactccaccgactacgtgttcgacggcgcgggcagcgtgcctttcgccgaggacgcgccgaccggcccgctgagcgtctacgggcagaccaagctggaaggcgagcaggccatccgcgccagcggctgccgccacctgatcttccgcaccagctgggtctacgccgcgcgcggcggaaacttcgccaagaccatgctgcgcctggccgggcaacgcgacgaactcaaggtcgtggccgaccagttcggcgcgcccaccagcgccgagctgatcgccgacgtcaccgcccaggccctgcagcgcctgtgctgggatgtcgagctggcagcacgggccagcggcacctaccacctggtcgccagcggcgagacgtcctggcacctctatgcgcgcttcgtcatcgaacaggcgctggagcggggctgggagttgcaggcgacgccgcagcgggtcctgccgatcgccaccgaggactacccggtgccggcgaagcgtccggccaattcgcgcctcgacaaccgcaagctgcaacaggtcttcggcctggtactgccagactggcgctaccatgccggacgcatgatccaggaactgagcgagcagggaccactatgaaacgcaagggcatcatcctcgccggaggctcgggcacccgcctgcacccggcaacgctggccatctccaagcagttgctgccggtgtacgacaagccgatgatctactacccgctcagtaccctgatgctggcgggcatccgcgagatactgatcatctcgaccccacaggacaccccacgcttccagcagttgctgggcgacggttcgaactggggcctggacctgcaatatgccgtgcaaccgtcgccggacggcctggcccaggccttcctgatcggcgagtcgttcatcggcaacgacctcagcgcgctggtcctgggcgacaacctctattacggccacgacttccacgagttgctcggcagcgcttcgcagcgccagaccggcgccagtgtcttcgcctaccacgtgctggacccggagcgctacggcgtggtcgagttcgaccagggcggcaaggccatcagcctggaagagaagccactggagccgaagtcgaactacgcggtcaccggcctgtatttctacgaccagcaggtggtggacatcgccagggacctgaagccttcgccgcgcggcgagctggagatcaccgacgtcaaccgcgcctatctggagcgcggccagctcagcgtggagatcatgggccgcggctacgcctggctggataccggcacccacgattcgctgctcgaggccggccagttcatcgccaccctggagaaccgccagggtctcaaggtggcctgcccggaagagatcgcctaccggcagaagtggatcgacgccgcgcaactggaaaaactcgccgcgccgctggccaagaacggctacggccaatacctcaagcgcctgctgaccgagaccgtgtactgatgaaagcgacccgcctggcaattcccgacgtcatcctcttcgaaccccgggtgttcggcgacgatcgcggattcttcttcgaaagctacaaccagcgcgccttcgaggaagcctgcggtcatccggtcagcttcgtccaggacaaccattcgcgttccgcccgtggcgtcctccgcggcctgcactaccagatccggcaagcccagggaaaactggtgcgcgccactctcggcgaggtattcgacgtggccgtcgacctgcgtcgcggctcgccgaccttcggccagtgggtaggcgaacgcctgagcgcggagaacaagcgccagatgtggattccggccggcttcgcgcacggcttcgtggtgctcagcgaatacgccgagttcctctacaagaccaccgacttctgggcgccggaacacgaacgctgcatcgtctggaacgatcccgagctgaagatcgactggccgctgcaggatgcccccctgctttcggagaaggaccgccagggcaaggcattcgccgacgccgactgcttcccctga

SEQ ID NO: 3 - DNA, 2028 bp, Artificial Sequence
  Promtor
ttaatctttctgcgaattgagatgacgccactggctgggcgtcatcccggtttcccgggtaaacaccaccgaaaaatagttactatcttcaaagccacattcggtcgaaatatcactgattaacaggcggctatgctggagaagatattgcgcatgacacactctgacctgtcgcagatattgattgatggtcattccagtctgctggcgaaattgctgacgcaaaacgcgctcactgcacgatgcctcatcacaaaatttatccagcgcaaagggacttttcaggctagccgccagccgggtaatcagcttatccagcaacgtttcgctggatgttggcggcaacgaatcactggtgtaacgatggcgattcagcaacatcaccaactgcccgaacagcaactcagccatttcgttagcaaacggcacatgctgactactttcatgctcaagctgaccgataacctgccgcgcctgcgccatccccatgctacctaagcgccagtgtggttgccctgcgctggcgttaaatcccggaatcgccccctgccagtcaagattcagcttcagacgctccgggcaataaataatattctgcaaaaccagatcgttaacggaagcgtaggagtgtttatcgtcagcatgaatgtaaaagagatcgccacgggtaatgcgataagggcgatcgttgagtacatgcaggccattaccgcgccagacaatcaccagctcacaaaaatcatgtgtatgttcagcaaagacatcttgcggataacggtcagccacagcgactgcctgctggtcgctggcaaaaaaatcatctttgagaagttttaactgatgcgccaccgtggctacctcggccagagaacgaagttgattattcgcaatatggcgtacaaatacgttgagaagattcgcgttattgcagaaagccatcccgtccctggcgaatatcacgcggtgaccagttaaactctcggcgaaaaagcgtcgaaaagtggttactgtcgctgaatccacagcgataggcgatgtcagtaacgctggcctcgctgtggcgtagcagatgtcgggctttcatcagtcgcaggcggttcaggtatcgctgaggcgtcagtcccgtttgctgcttaagctgccgatgtagcgtacgcagtgaaagagaaaattgatccgccacggcatcccaattcacctcatcggcaaaatggtcctccagccaggccagaagcaagttgagacgtgatgcgctgttttccaggttctcctgcaaactgcttttacgcagcaagagcagtaattgcataaacaagatctcgcgactggcggtcgagggtaaatcattttccccttcctgctgttccatctgtgcaaccagctgtcgcacctgctgcaatacgctgtggttaacgcgccagtgagacggatactgcccatccagctcttgtggcagcaactgattcagcccggcgagaaactgaaatcgatccggcgagcgatacagcacattggtcagacacagattatcggtatgttcatacagatgccgatcatgatcgcgtacgaaacagaccgtgccaccggtgatggtatagggctgcccattaaacacatgaatacccgtgccatgttcgacaatcacaatttcatgaaaatcatgatgatgttcaggaaaatccgcctgcgggagccggggttctatcgccacggacgcgttaccagacggaaaaaaatccacactatgtaatacggtcatactggcctcctgatgtcgtcaacacggcgaaatagtaatcacgaggtcaggttcttaccttaaattttcgacggaaaaccacgtaaaaaacgtcgatttttcaagatacagcgtgaattttcaggaaatgcggtgagcatcacatcaccacaattcagcaaattgtgaacatcatcacgttcatctttccctggttgccaatggcccattttcctgtcagtaacgagaaggtcgcgaattcaggcgctttttagactggtcgtaatgaa

SEQ ID NO: 4 - DNA, 1181 bp, Artificial Sequence
  Promotor
ttatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactg

SEQ ID NO: 5 - DNA, 107 bp, Artificial Sequence
  Terminator
caaataaaacgaaaggctcagtcgaaagactgggcctttcgttttatctgttgtttgtcggtgaacgctctcctgagtaggacaaatccgccgggagcggatttgaa

SEQ ID NO: 6 - DNA, 17337 bp, Artificial Sequence
  Vector
cacaaaattcctgcaggggccggcccagcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcaactagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggatttaaatttaatctttctgcgaattgagatgacgccactggctgggcgtcatcccggtttcccgggtaaacaccaccgaaaaatagttactatcttcaaagccacattcggtcgaaatatcactgattaacaggcggctatgctggagaagatattgcgcatgacacactctgacctgtcgcagatattgattgatggtcattccagtctgctggcgaaattgctgacgcaaaacgcgctcactgcacgatgcctcatcacaaaatttatccagcgcaaagggacttttcaggctagccgccagccgggtaatcagcttatccagcaacgtttcgctggatgttggcggcaacgaatcactggtgtaacgatggcgattcagcaacatcaccaactgcccgaacagcaactcagccatttcgttagcaaacggcacatgctgactactttcatgctcaagctgaccgataacctgccgcgcctgcgccatccccatgctacctaagcgccagtgtggttgccctgcgctggcgttaaatcccggaatcgccccctgccagtcaagattcagcttcagacgctccgggcaataaataatattctgcaaaaccagatcgttaacggaagcgtaggagtgtttatcgtcagcatgaatgtaaaagagatcgccacgggtaatgcgataagggcgatcgttgagtacatgcaggccattaccgcgccagacaatcaccagctcacaaaaatcatgtgtatgttcagcaaagacatcttgcggataacggtcagccacagcgactgcctgctggtcgctggcaaaaaaatcatctttgagaagttttaactgatgcgccaccgtggctacctcggccagagaacgaagttgattattcgcaatatggcgtacaaatacgttgagaagattcgcgttattgcagaaagccatcccgtccctggcgaatatcacgcggtgaccagttaaactctcggcgaaaaagcgtcgaaaagtggttactgtcgctgaatccacagcgataggcgatgtcagtaacgctggcctcgctgtggcgtagcagatgtcgggctttcatcagtcgcaggcggttcaggtatcgctgaggcgtcagtcccgtttgctgcttaagctgccgatgtagcgtacgcagtgaaagagaaaattgatccgccacggcatcccaattcacctcatcggcaaaatggtcctccagccaggccagaagcaagttgagacgtgatgcgctgttttccaggttctcctgcaaactgcttttacgcagcaagagcagtaattgcataaacaagatctcgcgactggcggtcgagggtaaatcattttccccttcctgctgttccatctgtgcaaccagctgtcgcacctgctgcaatacgctgtggttaacgcgccagtgagacggatactgcccatccagctcttgtggcagcaactgattcagcccggcgagaaactgaaatcgatccggcgagcgatacagcacattggtcagacacagattatcggtatgttcatacagatgccgatcatgatcgcgtacgaaacagaccgtgccaccggtgatggtatagggctgcccattaaacacatgaatacccgtgccatgttcgacaatcacaatttcatgaaaatcatgatgatgttcaggaaaatccgcctgcgggagccggggttctatcgccacggacgcgttaccagacggaaaaaaatccacactatgtaatacggtcatactggcctcctgatgtcgtcaacacggcgaaatagtaatcacgaggtcaggttcttaccttaaattttcgacggaaaaccacgtaaaaaacgtcgatttttcaagatacagcgtgaattttcaggaaatgcggtgagcatcacatcaccacaattcagcaaattgtgaacatcatcacgttcatctttccctggttgccaatggcccattttcctgtcagtaacgagaaggtcgcgaattcaggcgctttttagactggtcgtaatgaacatttaaatgaattcccttgggactctagagatccgcgggggcccaggaggggggatctggcatttttgggaggtgtgaaatgcggcgcgaaagtctgttggtatcggtttgcaagggcctgcgggtacatgtcgagcgcgttgggcaggatcccgggcgcagcacggtgatgctggtcaacggcgcgatggcgaccaccgcctcgttcgcccggacctgcaagtgcctggccgaacatttcaacgtggtgctgttcgacctgcccttcgccgggcagtcgcgtcagcacaacccgcagcgggggttgatcaccaaggacgacgaggtggaaatcctcctggcgctgatcgagcgcttcgaggtcaatcacctggtctccgcgtcctggggcggtatctccacgctgctggcgctgtcgcgcaatccgcgcggcatccgcagctcggtggtgatggcattcgcccctggactgaaccaggcgatgctcgactacgtcgggcgggcgcaggcgctgatcgagctggacgacaagtcggcgatcggccatctgctcaacgagaccgtcggcaaatacctgccgccgcgcctgaaagccagcaaccatcagcacatggcttcgctggccaccggcgaatacgagcaggcgcgctttcacatcgaccaggtgctggcgctcaacgatcggggctacctggcttgcctggagcggatccagagccacgtgcatttcatcaacggcagctgggacgaatacaccaccgccgaggacgcccgccagttccgcgactacctgccgcactgcagtttctcgcgggtggagggcaccgggcatttcctcgacctggagtccaagctggccgcggtacgcgtgcaccgcgccctgctcgagcacctgctgaagcaaccggagccgcagcgggcggaacgcgcggcgggattccacgagatggccatcggctacgcctgaacccttgacctgcgaagacccggcctggccgggctttgcggttgcataacgcacggagtagcaccatgcacgccatcctcatcgccatcggctcggccggcgacgtatttcccttcatcggcctggcccggaccctgaaattgcgcgggcaccgcgtgagcctctgcaccatcccggtgtttcgcgacgcggtggagcagcacggcatcgcgttcgtcccgctgagcgacgaactgacctaccgccggaccatgggcgatccgcgcctgtgggaccccaagacgtccttcggcgtgctctggcaaaccatcgccgggatgatcgagccggtctacgagtacgtctcggcgcagcgccatgacgacatcgtggtggtcggctcgctctgggcgctgggcgcacgcatcgctcacgagaagtacgggattccctacctgtccgcgcaggtctcgccatcgaccttgttgtcggcgcacctgccgccggtacaccccaagttcaacgtgcccgagcagatgccgctggcgatgcgcaagctgctctggcgctgcatcgagcgcttcaagctggatcgcacctgcgcgccggatatcaacgcggtgcggcgcaaggtcggcctggagacgccggtgaagcgcatcttcacccaatggatgcattcgccgcagggcgtggtctgcctgttcccggcctggttcgcgccgccccagcaggattggccgcaacccctgcacatgaccggcttcccgctgttcgacggcagtatcccggggaccccgctcgacgacgaactgcaacgctttctcgatcagggcagccggccgctggtgttcacccagggctcgaccgaacacctgcagggcgacttctacgccatggccctgcgcgcgctggaacgcctcggcgcgcgtgggatcttcctcaccggcgccggccaggaaccgctgcgcggcttgccgaaccacgtgctgcagcgcgcctacgcgccactgggagccttgctgccatcgtgcgccgggctggtccatccgggcggtatcggcgccatgagcctggccttggcggcgggggtgccgcaggtgctgctgccctgcgcccacgaccagttcgacaatgccgaacggctggtccggctcggctgcgggatgcgcctgggcgtgccattgcgcgagcaggagttgcgcggggcgctgtggcgcttgctcgaggacccggccatggcggcggcctgtcggcgtttcatggaattgtcacaaccgcacagtatcgcttgcggtaaagcggcccaggtggtcgaacgttgtcatagggagggggatgcgcgatggctgaaggctgcgtcctgacctacgggagaagaacgatcatggaccggatagacatgggcgtgctggtggtactgttcaatcctggcgacgacgacctggaacaccttggcgaactggcggcggcgtttccgcaactgcgcttccttgccgtcgacaactcaccgcacagcgatccgcagcgcaatgcccggctgcgcgggcaaggcatcgccgtgctgcaccacggcaaccggcagggcatcgccggcgccttcaaccagggactcgacgcgctattccggcgtggcgtgcagggtgtgctgctgctcgaccaggactcccgtcccggcggcgccttcctcgccgcccagtggcgcaacctgcaggcgcgcaacggtcaggcctgcctgctcggcccacggatcttcgaccggggtgaccggcgcttcctgccggccatccatctcgacggactgacgctcaggcaattgtctctggacggcctgacgaccccgcagcgcacctcgttcctgatctcctccggctgcctgctgacccgcgaggcctaccagcgcctcggccacttcgacgaggaactgttcatcgaccacgtggacaccgaatacagcctgcgcgcccaggcgctggacgtgcccctgtacgtcgacccgcggctggtcctcgagcaccgcatcggcacgcgcaagacccgccgcctcggcggtctcagcctcagcgcgatgaaccacgccccgctgcgccgctactacctggcgcgcaacggcctgctggtcctgcgccgctacgcccggtcctcgccgctggccctgctggcgaacctgccgaccctgacccagggcctcgcggtgctcctgctcgaacgcgacaagctgctcaagctgcgctgcctgggctggggcctgtgggacggcctgcggggacgcggcggcgcgctggagaccaaccgcccgcgcctgctgaagcgcctcgccggcccggccgtggcgtccgtagcttccggcaaggccaaggcctagtcggcgaaacgcattccctctagagtttaaacaccaggtgcgatcgcgcggccgcgctcgagcacgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgatgataagctgtcaaacatgagaattcttgaagacgaaagggcctcgtgtgtacaataatatttgcccatggatttaaataaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgatttaaatgaattcccttgggactcttaattaacaggaggaggtatgactcatgacgattctcgtgaccggcagcgccggcttcatcggcgccaatttcgtgctcgactggctggccctgcatgacgagccggtggtcagcctcgacaagctcacctacgccggcaaccggcagaacctcgccagcctcgacggcgacgcccggcacaccttcgtcgccggcgatatcggcgatagccagctggtagcccgcctgctcgccgagcaccagccgcgggcgatcctcaacttcgccgcggaatcccatgtggaccgctcgatccacggccccgaggacttcatccagaccaacatcgtcggcaccttccgcctgctggaagaagtgcgcgcctactggggcgcgctggagccggaagcgaaggcggcattccgcttcctccacgtctccaccgacgaagtctatggctcgctggcaccgagcgatccggccttcaccgagaacaaccgctacgagccgaacagtccctactcggcgtccaaggcggcctccgaccacctagtgcgggcctatcaccacacctatgggctgccggtgctgaccaccaactgctcgaacaactacggcccgtaccacttcccggaaaagctcatcccactggtgatccacaacgccctggccggcaagccgctgccgatctacggcgacggccagcagatccgcgactggctctacgtcaaggaccattgcagcgccatccgccgggtcctcgaagccgggcaactgggcgagacctacaatgtcggcggctggaacgaaaaggccaacctcgacgtggtcgagaccctctgcgccatcctcgaccaggagcagccgcgcgccgacggccgcagctatcgcgagcagatcaccttcgtcaaggatcgtccgggccatgatcgccgctacgccatcgatgccacgcgcctggagcgcgagctgggctggaagccggcggaaaccttcgagaccggcatccgcaagaccgtgcgctggtacctggacaaccaggactgggtggccaacgtaaccagcggtgcctaccgcgagtgggtgggtaagcagtacgcatgaaccggatccttctcctcggcgccaacggccaggtcggctgggagctgcagcgcgccctggcgccgctgggcgaactgctggtctgtgaccgtcggcgcgccgatctcgccgaccccgaaggcctggcgcgactggttcgcgccgagcggccgcagttcatcgtcaacgccggtgcctacaccgcggtggacaaggccgagagcgatgccgacaacgcccgcctgatcaatgcccgcgccgtcgcggtactggccgaggaggccgcggcctgcggcgcctggctggtgcattactccaccgactacgtgttcgacggcgcgggcagcgtgcctttcgccgaggacgcgccgaccggcccgctgagcgtctacgggcagaccaagctggaaggcgagcaggccatccgcgccagcggctgccgccacctgatcttccgcaccagctgggtctacgccgcgcgcggcggaaacttcgccaagaccatgctgcgcctggccgggcaacgcgacgaactcaaggtcgtggccgaccagttcggcgcgcccaccagcgccgagctgatcgccgacgtcaccgcccaggccctgcagcgcctgtgctgggatgtcgagctggcagcacgggccagcggcacctaccacctggtcgccagcggcgagacgtcctggcacctctatgcgcgcttcgtcatcgaacaggcgctggagcggggctgggagttgcaggcgacgccgcagcgggtcctgccgatcgccaccgaggactacccggtgccggcgaagcgtccggccaattcgcgcctcgacaaccgcaagctgcaacaggtcttcggcctggtactgccagactggcgctaccatgccggacgcatgatccaggaactgagcgagcagggaccactatgaaacgcaagggcatcatcctcgccggaggctcgggcacccgcctgcacccggcaacgctggccatctccaagcagttgctgccggtgtacgacaagccgatgatctactacccgctcagtaccctgatgctggcgggcatccgcgagatactgatcatctcgaccccacaggacaccccacgcttccagcagttgctgggcgacggttcgaactggggcctggacctgcaatatgccgtgcaaccgtcgccggacggcctggcccaggccttcctgatcggcgagtcgttcatcggcaacgacctcagcgcgctggtcctgggcgacaacctctattacggccacgacttccacgagttgctcggcagcgcttcgcagcgccagaccggcgccagtgtcttcgcctaccacgtgctggacccggagcgctacggcgtggtcgagttcgaccagggcggcaaggccatcagcctggaagagaagccactggagccgaagtcgaactacgcggtcaccggcctgtatttctacgaccagcaggtggtggacatcgccagggacctgaagccttcgccgcgcggcgagctggagatcaccgacgtcaaccgcgcctatctggagcgcggccagctcagcgtggagatcatgggccgcggctacgcctggctggataccggcacccacgattcgctgctcgaggccggccagttcatcgccaccctggagaaccgccagggtctcaaggtggcctgcccggaagagatcgcctaccggcagaagtggatcgacgccgcgcaactggaaaaactcgccgcgccgctggccaagaacggctacggccaatacctcaagcgcctgctgaccgagaccgtgtactgatgaaagcgacccgcctggcaattcccgacgtcatcctcttcgaaccccgggtgttcggcgacgatcgcggattcttcttcgaaagctacaaccagcgcgccttcgaggaagcctgcggtcatccggtcagcttcgtccaggacaaccattcgcgttccgcccgtggcgtcctccgcggcctgcactaccagatccggcaagcccagggaaaactggtgcgcgccactctcggcgaggtattcgacgtggccgtcgacctgcgtcgcggctcgccgaccttcggccagtgggtaggcgaacgcctgagcgcggagaacaagcgccagatgtggattccggccggcttcgcgcacggcttcgtggtgctcagcgaatacgccgagttcctctacaagaccaccgacttctgggcgccggaacacgaacgctgcatcgtctggaacgatcccgagctgaagatcgactggccgctgcaggatgcccccctgctttcggagaaggaccgccagggcaaggcattcgccgacgccgactgcttcccctgaacggcagggagcgaccggactcggcgcaaggcgtggtaatttagggtttaaacaccaggtgcgatcgcgcggccgcgctcgagcacgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgatgataagctgtcaaacatgagaattcttgaactagttgtacaaacgttcgtcaaaagggcga

SEQ ID NO: 7 - DNA, 27 bp, Artificial Sequence
  Primer
ccaatacatgcctgacgctaaaaaaca

SEQ ID NO: 8 - DNA, 28 bp, Artificial Sequence
  Primer
tagacgagttaatcgtgagcgcctattt

SEQ ID NO: 9 - DNA, 33 bp, Artificial Sequence
  Primer
gccgctttggtcccggccgccaaggtcattaac

SEQ ID NO: 10 - DNA, 28 bp, Artificial Sequence
  Primer
tcaggcatgtattggatcccgaggtagt

SEQ ID NO: 11 - DNA, 33 bp, Artificial Sequence
  Primer
gcttgcatgcctgcaggcgtgatgttgtacttc

SEQ ID NO: 12 - DNA, 27 bp, Artificial Sequence
  Primer
cgattaactcgtctacaccatcaataa

SEQ ID NO: 13 - DNA, 1410 bp, Artificial Sequence
  PCR product
ccaatacatgcctgacgctaaaaaacaggggcggtcaaacaaggcaatgacgtttttcgtctgcttccttgccgctctggcgggattactctttggcctggatatcggtgtaattgctggcgcactgccgtttattgcagatgaattccagattacttcgcacacgcaagaatgggtcgtaagctccatgatgttcggtgcggcagtcggtgcggtgggcagcggctggctctcctttaaactcgggcgcaaaaagagcctgatgatcggcgcaattttgtttgttgccggttcgctgttctctgcggctgcgccaaacgttgaagtactgattctttcccgcgttctactggggctggcggtgggtgtggcctcttataccgcaccgctgtacctctctgaaattgcgccggaaaaaattcgtggcagtatgatctcgatgtatcagttgatgatcactatcgggatcctcggtgcttatctttctgataccgccttcagctacaccggtgcatggcgctggatgctgggtgtgattatcatcccggcaattttgctgctgattggtgtcttcttcctgccagacagcccacgttggtttgccgccaaacgccgttttgttgatgccgaacgcgtgctgctacgcctgcgtgacaccagcgcggaagcgaaacgcgaactggatgaaatccgtgaaagtttgcaggttaaacagagtggctgggcgctgtttaaagagaacagcaacttccgccgcgcggtgttccttggcgtactgttgcaggtaatgcagcaattcaccgggatgaacgtcatcatgtattacgcgccgaaaatcttcgaactggcgggttataccaacactaccgagcaaatgtgggggaccgtgattgtcggcctgaccaacgtacttgccacctttatcgcaatcggccttgttgaccgctggggacgtaaaccaacgctaacgctgggcttcctggtgatggctgctggcatgggcgtactcggtacaatgatgcatatcggtattcactctccgtcggcgcagtatttcgccatcgccatgctgctgatgtttattgtcggttttgccatgagtgccggtccgctgatttgggtactgtgctccgaaattcagccgctgaaaggccgcgattttggcatcacctgctccactgccaccaactggattgccaacatgatcgttggcgcaacgttcctgaccatgctcaacacgctgggtaacgccaacaccttctgggtgtatgcggctctgaacgtactgtttatcctgctgacattgtggctggtaccggaaaccaaacacgtttcgctggaacatattgaacgtaatctgatgaaaggtcgtaaactgcgcgaaataggcgctcacgattaactcgtcta

SEQ ID NO: 14 - DNA, 675 bp, Artificial Sequence
  PCR product
gccgctttggtcccggccgccaaggtcattaacggcaaggccggcatgcagatcatgggcgactgggccaagagcgaatggaccctggcgaagaaaactgctggcaaggattaccagtgcgtgccgttccccggtactgacaagtccttcctgtacaacatcgactcgttggtggtgttcaagcagaacaacgctggcacctctgctggtcagcaggacatcgcccgcaaggtgttgggtgaggacttccagaaggtcttcagcatcaacaagggttcgatcccggtacgcaacgacatgcttgccgacatgggcaagtatggtttcgatgcctgtgctcaaacctccgccaaggacttcctggctgacgccaaaacgggcggcctgcaaccgagcatggcgcacaacatggccaccacgctggccgtgcagggcgcgttcttcgatgtggtgaccaactacatcaacgaccccaaggccgacccggccgatgcggcgaagaagctggcggcggctatcaaggctgcccagtaacggctgttcgcgggcacgcccgctcccagatgggccctgctcaccactgttcctggtcatgacgccggacctgtgggagcgggcttgcccgcgaagcggtgcgtagcaccgccactacctcgggatccaatacatgcctga

SEQ ID NO: 15 - DNA, 697 bp, Artificial Sequence
  PCR product
cgattaactcgtctacaccatcaataagaaaaagaggacgcaaagggatggaacagcgcaaacgcatcaagacactgggatcgttggccttgcttgcacttgtaggcagcagcggtacacaggctgccgaggctttttccagcgaatccaaatggatgaccggcgactggggcggcacccggaccgagctgctggacaagggctatgacttcaccctcgattatgtgggtgaggtggctggcaacctgcatggcggctacaacgacgacaagacggcacgctacagcgaccagttcgccctcggcgcgcatctggacttgcagaagatactgggctggcatgatgccgagttcaagctggcaatcaccgagcgaagcggtcgcaacctgtccaacgaccgcatcagcgacccgcgcgccgggcagttcagctcggtgcaggaggtgtggggccgtggccagacctggcgcctgacccagatgtggatcaagcagaagtacttcgacggcgcgctggacgtgaaatttggccgttttggcgagggcgaggacttcaacagcttcccttgcgacttccagaacctggccttctgcggctcgcaggtgggcaactgggtgggcggcatctggtacaactggccggtcagccagtgggcgctgcgggtgaagtacaacatcacgcctgcaggcatgcaagc

SEQ ID NO: 16 - DNA, 4831 bp, Artificial Sequence
  Vector
atggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgggctcgatcccctcgcgagttggttcagctgctgcctgaggctggacgacctcgcggagttctaccggcagtgcaaatccgtcggcatccaggaaaccagcagcggctatccgcgcatccatgcccccgaactgcaggagtggggaggcacgatggccgctttggtcccggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctttccttcttcactgtcccttctagaacgttctagaagggacagtgaagaaggaacacccgctcgcgggtgggcctactctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggtcaggcgtccttttgcttggtgccgaagatcttgtcaccggcatcacccaggcctggcacgatgtagccgtgctcgttcaggcgctggtcgatcgaggcggtgtagatcttcacgtccgggtgggctttttccaccacctcgatgccttctggtgcggcgaccagcaccatggcgcgaatctctttgcagccggcctttttcagcaggtcgatggtggcaaccatcgagccgccggtggccagcatcgggtcgatgatcagggccaggcgctggttgatgtccggcgcgagcttttccagataggtgtgggcttcgagggtttcttcgtttcgggcaacgccgacggcgctgaccttggcccccgggatcaggctgagcacgccgtcgagcatgccgatgccggcgcgcaggatcggtactacggtgatcttcttgccggcgattttttcaaccgagaccttgccacaccagccgtcgatctcgtaggtttcgaggggcaggtcctgggtggcttcatacgtcaggagcgcgccgacttcctgggcgagttcgcgaaaattcttggtgctgatatcggcacggcgcatcaggccaagcttgtggcggatcagcggatggcggatctcacgagtgggcataggggagggctccgaaaggcgggcaaaaaaaccgcgctagattaatctattcagcctgtgctgtcgtctggtcattctggacgttagtccataaatgcttgatctgtgacgagcggatgcgtacctttgcccgcttttccaaaatgctagccggctacgtatcgataagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctg

SEQ ID NO: 17 - DNA, 7535 bp, Artificial Sequence
  Vector
ctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggtcaggcgtccttttgcttggtgccgaagatcttgtcaccggcatcacccaggcctggcacgatgtagccgtgctcgttcaggcgctggtcgatcgaggcggtgtagatcttcacgtccgggtgggctttttccaccacctcgatgccttctggtgcggcgaccagcaccatggcgcgaatctctttgcagccggcctttttcagcaggtcgatggtggcaaccatcgagccgccggtggccagcatcgggtcgatgatcagggccaggcgctggttgatgtccggcgcgagcttttccagataggtgtgggcttcgagggtttcttcgtttcgggcaacgccgacggcgctgaccttggcccccgggatcaggctgagcacgccgtcgagcatgccgatgccggcgcgcaggatcggtactacggtgatcttcttgccggcgattttttcaaccgagaccttgccacaccagccgtcgatctcgtaggtttcgaggggcaggtcctgggtggcttcatacgtcaggagcgcgccgacttcctgggcgagttcgcgaaaattcttggtgctgatatcggcacggcgcatcaggccaagcttgtggcggatcagcggatggcggatctcacgagtgggcataggggagggctccgaaaggcgggcaaaaaaaccgcgctagattaatctattcagcctgtgctgtcgtctggtcattctggacgttagtccataaatgcttgatctgtgacgagcggatgcgtacctttgcccgcttttccaaaatgctagccggctacgtatcgataagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgggctcgatcccctcgcgagttggttcagctgctgcctgaggctggacgacctcgcggagttctaccggcagtgcaaatccgtcggcatccaggaaaccagcagcggctatccgcgcatccatgcccccgaactgcaggagtggggaggcacgatggccgctttggtcccggccgccaaggtcattaacggcaaggccggcatgcagatcatgggcgactgggccaagagcgaatggaccctggcgaagaaaactgctggcaaggattaccagtgcgtgccgttccccggtactgacaagtccttcctgtacaacatcgactcgttggtggtgttcaagcagaacaacgctggcacctctgctggtcagcaggacatcgcccgcaaggtgttgggtgaggacttccagaaggtcttcagcatcaacaagggttcgatcccggtacgcaacgacatgcttgccgacatgggcaagtatggtttcgatgcctgtgctcaaacctccgccaaggacttcctggctgacgccaaaacgggcggcctgcaaccgagcatggcgcacaacatggccaccacgctggccgtgcagggcgcgttcttcgatgtggtgaccaactacatcaacgaccccaaggccgacccggccgatgcggcgaagaagctggcggcggctatcaaggctgcccagtaacggctgttcgcgggcacgcccgctcccagatgggccctgctcaccactgttcctggtcatgacgccggacctgtgggagcgggcttgcccgcgaagcggtgcgtagcaccgccactacctcgggatccaatacatgcctgacgctaaaaaacaggggcggtcaaacaaggcaatgacgtttttcgtctgcttccttgccgctctggcgggattactctttggcctggatatcggtgtaattgctggcgcactgccgtttattgcagatgaattccagattacttcgcacacgcaagaatgggtcgtaagctccatgatgttcggtgcggcagtcggtgcggtgggcagcggctggctctcctttaaactcgggcgcaaaaagagcctgatgatcggcgcaattttgtttgttgccggttcgctgttctctgcggctgcgccaaacgttgaagtactgattctttcccgcgttctactggggctggcggtgggtgtggcctcttataccgcaccgctgtacctctctgaaattgcgccggaaaaaattcgtggcagtatgatctcgatgtatcagttgatgatcactatcgggatcctcggtgcttatctttctgataccgccttcagctacaccggtgcatggcgctggatgctgggtgtgattatcatcccggcaattttgctgctgattggtgtcttcttcctgccagacagcccacgttggtttgccgccaaacgccgttttgttgatgccgaacgcgtgctgctacgcctgcgtgacaccagcgcggaagcgaaacgcgaactggatgaaatccgtgaaagtttgcaggttaaacagagtggctgggcgctgtttaaagagaacagcaacttccgccgcgcggtgttccttggcgtactgttgcaggtaatgcagcaattcaccgggatgaacgtcatcatgtattacgcgccgaaaatcttcgaactggcgggttataccaacactaccgagcaaatgtgggggaccgtgattgtcggcctgaccaacgtacttgccacctttatcgcaatcggccttgttgaccgctggggacgtaaaccaacgctaacgctgggcttcctggtgatggctgctggcatgggcgtactcggtacaatgatgcatatcggtattcactctccgtcggcgcagtatttcgccatcgccatgctgctgatgtttattgtcggttttgccatgagtgccggtccgctgatttgggtactgtgctccgaaattcagccgctgaaaggccgcgattttggcatcacctgctccactgccaccaactggattgccaacatgatcgttggcgcaacgttcctgaccatgctcaacacgctgggtaacgccaacaccttctgggtgtatgcggctctgaacgtactgtttatcctgctgacattgtggctggtaccggaaaccaaacacgtttcgctggaacatattgaacgtaatctgatgaaaggtcgtaaactgcgcgaaataggcgctcacgattaactcgtctacaccatcaataagaaaaagaggacgcaaagggatggaacagcgcaaacgcatcaagacactgggatcgttggccttgcttgcacttgtaggcagcagcggtacacaggctgccgaggctttttccagcgaatccaaatggatgaccggcgactggggcggcacccggaccgagctgctggacaagggctatgacttcaccctcgattatgtgggtgaggtggctggcaacctgcatggcggctacaacgacgacaagacggcacgctacagcgaccagttcgccctcggcgcgcatctggacttgcagaagatactgggctggcatgatgccgagttcaagctggcaatcaccgagcgaagcggtcgcaacctgtccaacgaccgcatcagcgacccgcgcgccgggcagttcagctcggtgcaggaggtgtggggccgtggccagacctggcgcctgacccagatgtggatcaagcagaagtacttcgacggcgcgctggacgtgaaatttggccgttttggcgagggcgaggacttcaacagcttcccttgcgacttccagaacctggccttctgcggctcgcaggtgggcaactgggtgggcggcatctggtacaactggccggtcagccagtgggcgctgcgggtgaagtacaacatcacgcctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctttccttcttcactgtcccttctagaacgttctagaagggacagtgaagaaggaacacccgctcgcgggtgggcctactctaagaaaccattattatcatgacattaacctataaaaataggcgtatcacgaggccctttcgt

SEQ ID NO: 18 - DNA, 1140 bp, Artificial Sequence
  knock out construct
gcatcgcttcgttacctgacgaggcgggcacgctgtttagacttcatgctacgcaagcgatcgacttgcacggcataacaacaagaaaggtgctccgatgaattccacgcttcgtctcgctgccgcaatttcctttgcctcgttaatccccttgggtgcccaggctgctgacgccaaaggcagtgtcgaagtggtgcactggtggacctccggtggtgaaaaagcggcggtcgatgtgctcaaggcccaggtcgaaaaagacggcttcatctggaaggacggcgccgtcgccggcggcggtggtgcaacggccatgaccgtgctcaaaagccgcgcagtggccggcaacccgccgggcgtcgcgcagatcaaaggcccggacatccaggactgggcggccaccggcctgctcgacgccgatgtgctcaaggatgtggccaaggaaggaaagtgggactcgctgctcgacaagaaagtggccgacaccgtgaagtacgacggtgactacgttgccgtaccggtgaatatccaccgcatcaactggctgtggatcaaccccgaggtgttcaaaaaagccggcatcgacaaggcgcccaccaccctcgacgaattctacgccgccgccgacaagctcaaggctgccggtttcatcccgctcgcccatggtgggcaaccctggcaggacagcaccgtgttcgaaagcgtggtgctgtcggtgatgggcgtcgatggctacaagaaggccttggtcgacctcgacagcgcaacgctgaccgggccgcagatggtcaaggcgctgaccgagttgaagaaagtcgccacctacatggacccggacggcaagggccaggactggaacctggaagccgccaaggtcattaacggcaaggccggcatgcagatcatgggcgactgggccaagagcgaatggaccctggcgaagaaaactgctggcaaggattaccagtgcgtgccgttccccggtactgacaagtccttcctgtacaacatcgactcgttggtggtgttcaagcagaacaacgctggcacctctgctggtcagcaggacatcgcccgcaaggtgttgggtgaggacttccagaaggtcttcagcatcaacaagggttcgatcccggtacg

SEQ ID NO: 19 - DNA, 39 bp, Artificial Sequence
  Primer
tacctcgggatccaatacatgtccagcgagtcgtcccag

SEQ ID NO: 20 - DNA, 22 bp, Artificial Sequence
  Primer
ttacttctgcgagcgccacatc

SEQ ID NO: 21 - DNA, 22 bp, Artificial Sequence
  Primer
catgtattggatcccgaggtag

SEQ ID NO: 22 - DNA, 47 bp, Artificial Sequence
  Primer
gctcgcagaagtaacaatacctcgtctacaccatcaataagaaaaag

SEQ ID NO: 23 - DNA, 1440 bp, Artificial Sequence
  PCR product
tacctcgggatccaatacatgtccagcgagtcgtcccagggcttggtcacccgcttggcgttgattgcagcgatcggcggtttgctcttcggctatgacagcgccgtcattgctgctatcgggacgccggtggacatccacttcatcgcccctcgccatctgtccgccaccgctgcggcctccctgagcggcatggtagttgtcgctgtgttggtcgggtgcgtgacgggctcgttgctgtcgggctggatcggcattcggtttggccgtcgtggtggcctgctcatgtcctccatctgtttcgtggctgccggctttggcgctgcactgacggagaagctgtttggcacgggtggctccgccctccaaatcttttgctttttccggtttctcgccgggttgggcattggtgtggtttccaccctcaccccgacctatatcgccgagatctgtccgcctgacaagcgtgggcagatggtatccggccagcaaatggctattgtaaccggtgccctgaccggctacatctttacctggctgttggcccacttcgggagcatcgactgggtgaacgccagcgggtggtgctggagcccagccagcgaaggcttgattgggattgccttcctcctcctgttgctgaccgcgccagatacccctcattggttggtgatgaagggtcgccatagcgaggctagcaaaattctggcacgcctcgagccacaggctgaccccaacctgacgattcagaaaatcaaagcaggcttcgacaaggccatggacaagagctcggcagggctgttcgcgttcggcatcaccgtggtctttgccggtgtctcggttgccgcgttccagcagctggtcggtattaacgccgttctgtactatgccccgcaaatgttccaaaatctgggtttcggtgctgataccgcgctgctccaaacgatctcgattggggttgtgaatttcatcttcacgatgatcgcgagccgcgtggtagaccgcttcggtcgcaagccgctgctgatttggggtgctctggggatggcggccatgatggccgtgttgggttgttgtttttggtttaaggtggggggtgttctgccgttggctagcgtgctgctgtacatcgcagtgttcggtatgtcgtggggccctgtgtgctgggtcgtactgagcgagatgttcccaagctcgatcaagggtgccgcgatgcccattgcggtcaccggtcaatggctcgccaatatcttggtgaacttcctcttcaaggtagccgacggttcccccgcactcaaccagacgttcaaccacggcttcagctacctggtgttcgctgccttgagcatcctgggtgggctcattgtggcccggttcgtcccggagaccaaggggcgcagcttggacgagattgaggagatgtggcgctcgcagaagtaa

SEQ ID NO: 24 - DNA, 670 bp, Artificial Sequence
  PCR product
gccgctttggtcccggccgccaaggtcattaacggcaaggccggcatgcagatcatgggcgactgggccaagagcgaatggaccctggcgaagaaaactgctggcaaggattaccagtgcgtgccgttccccggtactgacaagtccttcctgtacaacatcgactcgttggtggtgttcaagcagaacaacgctggcacctctgctggtcagcaggacatcgcccgcaaggtgttgggtgaggacttccagaaggtcttcagcatcaacaagggttcgatcccggtacgcaacgacatgcttgccgacatgggcaagtatggtttcgatgcctgtgctcaaacctccgccaaggacttcctggctgacgccaaaacgggcggcctgcaaccgagcatggcgcacaacatggccaccacgctggccgtgcagggcgcgttcttcgatgtggtgaccaactacatcaacgaccccaaggccgacccggccgatgcggcgaagaagctggcggcggctatcaaggctgcccagtaacggctgttcgcgggcacgcccgctcccagatgggccctgctcaccactgttcctggtcatgacgccggacctgtgggagcgggcttgcccgcgaagcggtgcgtagcaccgccactacctcgggatccaatacatg

SEQ ID NO: 25 - DNA, 710 bp, Artificial Sequence
  PCR product
gctcgcagaagtaacaatacctcgtctacaccatcaataagaaaaagaggacgcaaagggatggaacagcgcaaacgcatcaagacactgggatcgttggccttgcttgcacttgtaggcagcagcggtacacaggctgccgaggctttttccagcgaatccaaatggatgaccggcgactggggcggcacccggaccgagctgctggacaagggctatgacttcaccctcgattatgtgggtgaggtggctggcaacctgcatggcggctacaacgacgacaagacggcacgctacagcgaccagttcgccctcggcgcgcatctggacttgcagaagatactgggctggcatgatgccgagttcaagctggcaatcaccgagcgaagcggtcgcaacctgtccaacgaccgcatcagcgacccgcgcgccgggcagttcagctcggtgcaggaggtgtggggccgtggccagacctggcgcctgacccagatgtggatcaagcagaagtacttcgacggcgcgctggacgtgaaatttggccgttttggcgagggcgaggacttcaacagcttcccttgcgacttccagaacctggccttctgcggctcgcaggtgggcaactgggtgggcggcatctggtacaactggccggtcagccagtgggcgctgcgggtgaagtacaacatcacgcctgcaggcatgcaagc

SEQ ID NO: 26 - DNA, 7568 bp, Artificial Sequence
  vector
atggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgggctcgatcccctcgcgagttggttcagctgctgcctgaggctggacgacctcgcggagttctaccggcagtgcaaatccgtcggcatccaggaaaccagcagcggctatccgcgcatccatgcccccgaactgcaggagtggggaggcacgatggccgctttggtcccggccgccaaggtcattaacggcaaggccggcatgcagatcatgggcgactgggccaagagcgaatggaccctggcgaagaaaactgctggcaaggattaccagtgcgtgccgttccccggtactgacaagtccttcctgtacaacatcgactcgttggtggtgttcaagcagaacaacgctggcacctctgctggtcagcaggacatcgcccgcaaggtgttgggtgaggacttccagaaggtcttcagcatcaacaagggttcgatcccggtacgcaacgacatgcttgccgacatgggcaagtatggtttcgatgcctgtgctcaaacctccgccaaggacttcctggctgacgccaaaacgggcggcctgcaaccgagcatggcgcacaacatggccaccacgctggccgtgcagggcgcgttcttcgatgtggtgaccaactacatcaacgaccccaaggccgacccggccgatgcggcgaagaagctggcggcggctatcaaggctgcccagtaacggctgttcgcgggcacgcccgctcccagatgggccctgctcaccactgttcctggtcatgacgccggacctgtgggagcgggcttgcccgcgaagcggtgcgtagcaccgccactacctcgggatccaatacatgtccagcgagtcgtcccagggcttggtcacccgcttggcgttgattgcagcgatcggcggtttgctcttcggctatgacagcgccgtcattgctgctatcgggacgccggtggacatccacttcatcgcccctcgccatctgtccgccaccgctgcggcctccctgagcggcatggtagttgtcgctgtgttggtcgggtgcgtgacgggctcgttgctgtcgggctggatcggcattcggtttggccgtcgtggtggcctgctcatgtcctccatctgtttcgtggctgccggctttggcgctgcactgacggagaagctgtttggcacgggtggctccgccctccaaatcttttgctttttccggtttctcgccgggttgggcattggtgtggtttccaccctcaccccgacctatatcgccgagatctgtccgcctgacaagcgtgggcagatggtatccggccagcaaatggctattgtaaccggtgccctgaccggctacatctttacctggctgttggcccacttcgggagcatcgactgggtgaacgccagcgggtggtgctggagcccagccagcgaaggcttgattgggattgccttcctcctcctgttgctgaccgcgccagatacccctcattggttggtgatgaagggtcgccatagcgaggctagcaaaattctggcacgcctcgagccacaggctgaccccaacctgacgattcagaaaatcaaagcaggcttcgacaaggccatggacaagagctcggcagggctgttcgcgttcggcatcaccgtggtctttgccggtgtctcggttgccgcgttccagcagctggtcggtattaacgccgttctgtactatgccccgcaaatgttccaaaatctgggtttcggtgctgataccgcgctgctccaaacgatctcgattggggttgtgaatttcatcttcacgatgatcgcgagccgcgtggtagaccgcttcggtcgcaagccgctgctgatttggggtgctctggggatggcggccatgatggccgtgttgggttgttgtttttggtttaaggtggggggtgttctgccgttggctagcgtgctgctgtacatcgcagtgttcggtatgtcgtggggccctgtgtgctgggtcgtactgagcgagatgttcccaagctcgatcaagggtgccgcgatgcccattgcggtcaccggtcaatggctcgccaatatcttggtgaacttcctcttcaaggtagccgacggttcccccgcactcaaccagacgttcaaccacggcttcagctacctggtgttcgctgccttgagcatcctgggtgggctcattgtggcccggttcgtcccggagaccaaggggcgcagcttggacgagattgaggagatgtggcgctcgcagaagtaacaatacctcgtctacaccatcaataagaaaaagaggacgcaaagggatggaacagcgcaaacgcatcaagacactgggatcgttggccttgcttgcacttgtaggcagcagcggtacacaggctgccgaggctttttccagcgaatccaaatggatgaccggcgactggggcggcacccggaccgagctgctggacaagggctatgacttcaccctcgattatgtgggtgaggtggctggcaacctgcatggcggctacaacgacgacaagacggcacgctacagcgaccagttcgccctcggcgcgcatctggacttgcagaagatactgggctggcatgatgccgagttcaagctggcaatcaccgagcgaagcggtcgcaacctgtccaacgaccgcatcagcgacccgcgcgccgggcagttcagctcggtgcaggaggtgtggggccgtggccagacctggcgcctgacccagatgtggatcaagcagaagtacttcgacggcgcgctggacgtgaaatttggccgttttggcgagggcgaggacttcaacagcttcccttgcgacttccagaacctggccttctgcggctcgcaggtgggcaactgggtgggcggcatctggtacaactggccggtcagccagtgggcgctgcgggtgaagtacaacatcacgcctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctttccttcttcactgtcccttctagaacgttctagaagggacagtgaagaaggaacacccgctcgcgggtgggcctactctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggtcaggcgtccttttgcttggtgccgaagatcttgtcaccggcatcacccaggcctggcacgatgtagccgtgctcgttcaggcgctggtcgatcgaggcggtgtagatcttcacgtccgggtgggctttttccaccacctcgatgccttctggtgcggcgaccagcaccatggcgcgaatctctttgcagccggcctttttcagcaggtcgatggtggcaaccatcgagccgccggtggccagcatcgggtcgatgatcagggccaggcgctggttgatgtccggcgcgagcttttccagataggtgtgggcttcgagggtttcttcgtttcgggcaacgccgacggcgctgaccttggcccccgggatcaggctgagcacgccgtcgagcatgccgatgccggcgcgcaggatcggtactacggtgatcttcttgccggcgattttttcaaccgagaccttgccacaccagccgtcgatctcgtaggtttcgaggggcaggtcctgggtggcttcatacgtcaggagcgcgccgacttcctgggcgagttcgcgaaaattcttggtgctgatatcggcacggcgcatcaggccaagcttgtggcggatcagcggatggcggatctcacgagtgggcataggggagggctccgaaaggcgggcaaaaaaaccgcgctagattaatctattcagcctgtgctgtcgtctggtcattctggacgttagtccataaatgcttgatctgtgacgagcggatgcgtacctttgcccgcttttccaaaatgctagccggctacgtatcgataagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctg

SEQ ID NO: 27 - DNA, 4630 bp, Artificial Sequence
  knockout costruct
gcatcgcttcgttacctgacgaggcgggcacgctgtttagacttcatgctacgcaagcgatcgacttgcacggcataacaacaagaaaggtgctccgatgaattccacgcttcgtctcgctgccgcaatttcctttgcctcgttaatccccttgggtgcccaggctgctgacgccaaaggcagtgtcgaagtggtgcactggtggacctccggtggtgaaaaagcggcggtcgatgtgctcaaggcccaggtcgaaaaagacggcttcatctggaaggacggcgccgtcgccggcggcggtggtgcaacggccatgaccgtgctcaaaagccgcgcagtggccggcaacccgccgggcgtcgcgcagatcaaaggcccggacatccaggactgggcggccaccggcctgctcgacgccgatgtgctcaaggatgtggccaaggaaggaaagtgggactcgctgctcgacaagaaagtggccgacaccgtgaagtacgacggtgactacgttgccgtaccggtgaatatccaccgcatcaactggctgtggatcaaccccgaggtgttcaaaaaagccggcatcgacaaggcgcccaccaccctcgacgaattctacgccgccgccgacaagctcaaggctgccggtttcatcccgctcgcccatggtgggcaaccctggcaggacagcaccgtgttcgaaagcgtggtgctgtcggtgatgggcgtcgatggctacaagaaggccttggtcgacctcgacagcgcaacgctgaccgggccgcagatggtcaaggcgctgaccgagttgaagaaagtcgccacctacatggacccggacggcaagggccaggactggaacctggaagccgccaaggtcattaacggcaaggccggcatgcagatcatgggcgactgggccaagagcgaatggaccctggcgaagaaaactgctggcaaggattaccagtgcgtgccgttccccggtactgacaagtccttcctgtacaacatcgactcgttggtggtgttcaagcagaacaacgctggcacctctgctggtcagcaggacatcgcccgcaaggtgttgggtgaggacttccagaaggtcttcagcatcaacaagggttcgatcccggtacgcaacgacatgcttgccgacatgggcaagtatggtttcgatgcctgtgctcaaacctccgccaaggacttcctggctgacgccaaaacgggcggcctgcaaccgagcatggcgcacaacatggccaccacgctggccgtgcagggcgcgttcttcgatgtggtgaccaactacatcaacgaccccaaggccgacccggccgatgcggcgaagaagctggcggcggctatcaaggctgcccagtaacggctgttcgcgggcacgcccgctcccagatgggccctgctcaccactgttcctggtcatgacgccggacctgtgggagcgggcttgcccgcgaagcggtgcgtagcaccgccactacctcgggatccaatacatgtccagcgagtcgtcccagggcttggtcacccgcttggcgttgattgcagcgatcggcggtttgctcttcggctatgacagcgccgtcattgctgctatcgggacgccggtggacatccacttcatcgcccctcgccatctgtccgccaccgctgcggcctccctgagcggcatggtagttgtcgctgtgttggtcgggtgcgtgacgggctcgttgctgtcgggctggatcggcattcggtttggccgtcgtggtggcctgctcatgtcctccatctgtttcgtggctgccggctttggcgctgcactgacggagaagctgtttggcacgggtggctccgccctccaaatcttttgctttttccggtttctcgccgggttgggcattggtgtggtttccaccctcaccccgacctatatcgccgagatctgtccgcctgacaagcgtgggcagatggtatccggccagcaaatggctattgtaaccggtgccctgaccggctacatctttacctggctgttggcccacttcgggagcatcgactgggtgaacgccagcgggtggtgctggagcccagccagcgaaggcttgattgggattgccttcctcctcctgttgctgaccgcgccagatacccctcattggttggtgatgaagggtcgccatagcgaggctagcaaaattctggcacgcctcgagccacaggctgaccccaacctgacgattcagaaaatcaaagcaggcttcgacaaggccatggacaagagctcggcagggctgttcgcgttcggcatcaccgtggtctttgccggtgtctcggttgccgcgttccagcagctggtcggtattaacgccgttctgtactatgccccgcaaatgttccaaaatctgggtttcggtgctgataccgcgctgctccaaacgatctcgattggggttgtgaatttcatcttcacgatgatcgcgagccgcgtggtagaccgcttcggtcgcaagccgctgctgatttggggtgctctggggatggcggccatgatggccgtgttgggttgttgtttttggtttaaggtggggggtgttctgccgttggctagcgtgctgctgtacatcgcagtgttcggtatgtcgtggggccctgtgtgctgggtcgtactgagcgagatgttcccaagctcgatcaagggtgccgcgatgcccattgcggtcaccggtcaatggctcgccaatatcttggtgaacttcctcttcaaggtagccgacggttcccccgcactcaaccagacgttcaaccacggcttcagctacctggtgttcgctgccttgagcatcctgggtgggctcattgtggcccggttcgtcccggagaccaaggggcgcagcttggacgagattgaggagatgtggcgctcgcagaagtaacaatacctcgtctacaccatcaataagaaaaagaggacgcaaagggatggaacagcgcaaacgcatcaagacactgggatcgttggccttgcttgcacttgtaggcagcagcggtacacaggctgccgaggctttttccagcgaatccaaatggatgaccggcgactggggcggcacccggaccgagctgctggacaagggctatgacttcaccctcgattatgtgggtgaggtggctggcaacctgcatggcggctacaacgacgacaagacggcacgctacagcgaccagttcgccctcggcgcgcatctggacttgcagaagatactgggctggcatgatgccgagttcaagctggcaatcaccgagcgaagcggtcgcaacctgtccaacgaccgcatcagcgacccgcgcgccgggcagttcagctcggtgcaggaggtgtggggccgtggccagacctggcgcctgacccagatgtggatcaagcagaagtacttcgacggcgcgctggacgtgaaatttggccgttttggcgagggcgaggacttcaacagcttcccttgcgacttccagaacctggccttctgcggctcgcaggtgggcaactgggtgggcggcatctggtacaactggccggtcagccagtgggcgctgcgggtgaagtacaacatcacgccggagttcttcgtacaggtcggggccttcgagcagaacccttccaacctggaaaccggcaacggcttcaagctcagcggcagtggtaccaagggggcgatcttgccggtggaagcggtgtggtcgcccaaggtcaatggcctgccgggcgagtaccgcctgggttactactacagcacggccaaggctgacgatgtgtacgacgacgtcaacggcaacccgcaggcgctgacaggtgaagccttcaagtcgcactccagcaagcacggatggtgggtggtggcgcagcagcaggtcactgcccatggcggcgacgtcaaccggggcctcagcctgttcgccaacttcaccgtgcacgacaaggccaccaacgtggtcgacaactaccagcaggtggggctggtctacaaaggcgctttcgacgcccggcccaaggatgacatcggcttcggcgtggcgcgtattcatgtgaatgacgacgtgaagaagcgcgccgaactgctcaacgcacagagcggcatcaacgattacgacaaccctggtttcgtgccgctgcagcgtaccgaatacaacgcagagctctactacggcttccacgttaccaactggctgaccgtgaggcccaacctgcagtacatcaagagccctggcggggtggacgaggtggataacgcgctggtcgctggcttgaagattcagtcgtcattctgaggcaacttgttgtaaatttacgccaatccattttcggttcccggcacccactggtctgcagtgggtgttggggataggccgagacagcgctatctcaaacaacaagagctctgaaccatgcccgaacatccgctccatcgcttcttctcctcgcagcggccccggccgacattcgagtgggagcgttatcagcagcgcgatgtcctgatcatcgaccacccccgttgccaggcggtgttcagccgccagggcgcgcagttgctgcactttcaaccagcgggtgaacggccgtggctgtggt

SEQ ID NO: 28 - DNA, 29 bp, Artificial Sequence
  primer
tccaatacatgttccagcaagaagttacc

SEQ ID NO: 29 - DNA, 29 bp, Artificial Sequence
  primer
cctgagtttacttcttgatgcggataacc

SEQ ID NO: 30 - DNA, 28 bp, Artificial Sequence
  primer
agaagtaaactcaggagcactctcaatt

SEQ ID NO: 31 - DNA, 28 bp, Artificial Sequence
  primer
agacgagttagtggttacggatgtactc

SEQ ID NO: 32 - DNA, 28 bp, Artificial Sequence
  primer
ggaacatgtattggatcccgaggtagtg

SEQ ID NO: 33 - DNA, 29 bp, Artificial Sequence
  primer
accactaactcgtctacaccatcaataag

SEQ ID NO: 34 - DNA, 2595 bp, Artificial Sequence
  PCR product
tccaatacatgttccagcaagaagttaccattaccgctccgaacggtctgcacacccgccctgctgcccagtttgtaaaagaagctaagggcttcacttctgaaattactgtgacttccaacggcaaaagcgccagcgcgaaaagcctgtttaaactgcagactctgggcctgactcaaggtaccgttgtgactatctccgcagaaggcgaagacgagcagaaagcggttgaacatctggttaaactgatggcggaactcgagtaatttcccgggttcttttaaaaatcagtcacaagtaaggtagggttatgatttcaggcattttagcatccccgggtatcgctttcggtaaagctctgcttctgaaagaagacgaaattgtcattgaccggaaaaaaatttctgccgaccaggttgatcaggaagttgaacgttttctgagcggtcgtgccaaggcatcagcccagctggaaacgatcaaaacgaaagctggtgaaacgttcggtgaagaaaaagaagccatctttgaagggcatattatgctgctcgaagatgaggagctggagcaggaaatcatagccctgattaaagataagcacatgacagctgacgcagctgctcatgaagttatcgaaggtcaggcttctgccctggaagagctggatgatgaatacctgaaagaacgtgcggctgacgtacgtgatatcggtaagcgcctgctgcgcaacatcctgggcctgaagattatcgacctgagcgccattcaggatgaagtcattctggttgccgctgacctgacgccgtccgaaaccgcacagctgaacctgaagaaggtgctgggtttcatcaccgacgcgggtggccgtacttcccacacctctatcatggcgcgttctctggaactacctgctatcgtgggtaccggtagcgtcacctctcaggtgaaaaatgacgactatctgattctggatgccgtaaataatcaggtttacgtcaatccaaccaacgaagttattgataaaatgcgcgctgttcaggagcaagtggcttctgaaaaagcagagcttgctaaactgaaagatctgccagctattacgctggacggtcaccaggtagaagtatgcgctaacattggtacggttcgtgacgttgaaggtgcagagcgtaacggcgctgaaggcgttggtctgtatcgtactgagttcctgttcatggaccgcgacgcactgcccactgaagaagaacagtttgctgcttacaaagcagtggctgaagcgtgtggctcgcaagcggttatcgttcgtaccatggacatcggcggcgacaaagagctgccatacatgaacttcccgaaagaagagaacccgttcctcggctggcgcgctatccgtatcgcgatggatcgtagagagatcctgcgcgatcagctccgcgctatcctgcgtgcctcggctttcggtaaattgcgcattatgttcccgatgatcatctctgttgaagaagtgcgtgcactgcgcaaagagatcgaaatctacaaacaggaactgcgcgacgaaggtaaagcgtttgacgagtcaattgaaatcggcgtaatggtggaaacaccggctgccgcaacaattgcacgtcatttagccaaagaagttgatttctttagtatcggcaccaatgatttaacgcagtacactctggcagttgaccgtggtaatgatatgatttcacacctttaccagccaatgtcaccgtccgtgctgaacttgatcaagcaagttattgatgcttctcatgctgaaggcaaatggactggcatgtgtggtgagcttgctggcgatgaacgtgctacacttctgttgctggggatgggtctggacgaattctctatgagcgccatttctatcccgcgcattaagaagattatccgtaacacgaacttcgaagatgcgaaggtgttagcagagcaggctcttgctcaaccgacaacggacgagttaatgacgctggttaacaagttcattgaagaaaaaacaatctgctaatccacgagatgcggcccaatttactgcttaggagaagatcatgggtttgttcgataaactgaaatctctggtttccgacgacaagaaggataccggaactattgagatcattgctccgctctctggcgagatcgtcaatatcgaagacgtgccggatgtcgtttttgcggaaaaaatcgttggtgatggtattgctatcaaaccaacgggtaacaaaatggtcgcgccagtagacggcaccattggtaaaatctttgaaaccaaccacgcattctctatcgaatctgatagcggcgttgaactgttcgtccacttcggtatcgacaccgttgaactgaaaggcgaaggcttcaagcgtattgctgaagaaggtcagcgcgtgaaagttggcgatactgtcattgaatttgatctgccgctgctggaagagaaagccaagtctaccctgactccggttgttatctccaacatggacgaaatcaaagaactgatcaaactgtccggtagcgtaaccgtgggtgaaaccccggttatccgcatcaagaagtaaactcagg

SEQ ID NO: 35 - DNA, 1469 bp, Artificial Sequence
  PCR product
agaagtaaactcaggagcactctcaattatgtttaagaatgcatttgctaacctgcaaaaggtcggtaaatcgctgatgctgccggtatccgtactgcctatcgcaggtattctgctgggcgtcggttccgcgaatttcagctggctgcccgccgttgtatcgcatgttatggcagaagcaggcggttccgtctttgcaaacatgccactgatttttgcgatcggtgtcgccctcggctttaccaataacgatggcgtatccgcgctggccgcagttgttgcctatggcatcatggttaaaaccatggccgtggttgcgccactggtactgcatttacctgctgaagaaatcgcctctaaacacctggcggatactggcgtactcggagggattatctccggtgcgatcgcagcgtacatgtttaaccgtttctaccgtattaagctgcctgagtatcttggcttctttgccggtaaacgctttgtgccgatcatttctggcctggctgccatctttactggcgttgtgctgtccttcatttggccgccgattggttctgcaatccagaccttctctcagtgggctgcttaccagaacccggtagttgcgtttggcatttacggtttcatcgaacgttgcctggtaccgtttggtctgcaccacatctggaacgtacctttccagatgcagattggtgaatacaccaacgcagcaggtcaggttttccacggcgacattccgcgttatatggcgggtgacccgactgcgggtaaactgtctggtggcttcctgttcaaaatgtacggtctgccagctgccgcaattgctatctggcactctgctaaaccagaaaaccgcgcgaaagtgggcggtattatgatctccgcggcgctgacctcgttcctgaccggtatcaccgagccgatcgagttctccttcatgttcgttgcgccgatcctgtacatcatccacgcgattctggcaggcctggcattcccaatctgtattcttctggggatgcgtgacggtacgtcgttctcgcacggtctgatcgacttcatcgttctgtctggtaacagcagcaaactgtggctgttcccgatcgtcggtatcggttatgcgattgtttactacaccatcttccgcgtgctgattaaagcactggatctgaaaacgccgggtcgtgaagacgcgactgaagatgcaaaagcgacaggtaccagcgaaatggcaccggctctggttgctgcatttggtggtaaagaaaacattactaacctcgacgcatgtattacccgtctgcgcgtcagcgttgctgatgtgtctaaagtggatcaggccggcctgaagaaactgggcgcagcgggcgtagtggttgctggttctggtgttcaggcgattttcggtactaaatccgataacctgaaaaccgagatggatgagtacatccgtaaccactaactcgtct

SEQ ID NO: 36 - DNA, 674 bp, Artificial Sequence
  PCR product
gccgctttggtcccggccgccaaggtcattaacggcaaggccggcatgcagatcatgggcgactgggccaagagcgaatggaccctggcgaagaaaactgctggcaaggattaccagtgcgtgccgttccccggtactgacaagtccttcctgtacaacatcgactcgttggtggtgttcaagcagaacaacgctggcacctctgctggtcagcaggacatcgcccgcaaggtgttgggtgaggacttccagaaggtcttcagcatcaacaagggttcgatcccggtacgcaacgacatgcttgccgacatgggcaagtatggtttcgatgcctgtgctcaaacctccgccaaggacttcctggctgacgccaaaacgggcggcctgcaaccgagcatggcgcacaacatggccaccacgctggccgtgcagggcgcgttcttcgatgtggtgaccaactacatcaacgaccccaaggccgacccggccgatgcggcgaagaagctggcggcggctatcaaggctgcccagtaacggctgttcgcgggcacgcccgctcccagatgggccctgctcaccactgttcctggtcatgacgccggacctgtgggagcgggcttgcccgcgaagcggtgcgtagcaccgccactacctcgggatccaatacatgttcc

SEQ ID NO: 37 - DNA, 698 bp, Artificial Sequence
  PCR product
accactaactcgtctacaccatcaataagaaaaagaggacgcaaagggatggaacagcgcaaacgcatcaagacactgggatcgttggccttgcttgcacttgtaggcagcagcggtacacaggctgccgaggctttttccagcgaatccaaatggatgaccggcgactggggcggcacccggaccgagctgctggacaagggctatgacttcaccctcgattatgtgggtgaggtggctggcaacctgcatggcggctacaacgacgacaagacggcacgctacagcgaccagttcgccctcggcgcgcatctggacttgcagaagatactgggctggcatgatgccgagttcaagctggcaatcaccgagcgaagcggtcgcaacctgtccaacgaccgcatcagcgacccgcgcgccgggcagttcagctcggtgcaggaggtgtggggccgtggccagacctggcgcctgacccagatgtggatcaagcagaagtacttcgacggcgcgctggacgtgaaatttggccgttttggcgagggcgaggacttcaacagcttcccttgcgacttccagaacctggccttctgcggctcgcaggtgggcaactgggtgggcggcatctggtacaactggccggtcagccagtgggcgctgcgggtgaagtacaacatcacgcctgcaggcatgcaagc

SEQ ID NO: 38 - DNA, 10174 bp, Artificial Sequence
  vector
atggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgggctcgatcccctcgcgagttggttcagctgctgcctgaggctggacgacctcgcggagttctaccggcagtgcaaatccgtcggcatccaggaaaccagcagcggctatccgcgcatccatgcccccgaactgcaggagtggggaggcacgatggccgctttggtcccggccgccaaggtcattaacggcaaggccggcatgcagatcatgggcgactgggccaagagcgaatggaccctggcgaagaaaactgctggcaaggattaccagtgcgtgccgttccccggtactgacaagtccttcctgtacaacatcgactcgttggtggtgttcaagcagaacaacgctggcacctctgctggtcagcaggacatcgcccgcaaggtgttgggtgaggacttccagaaggtcttcagcatcaacaagggttcgatcccggtacgcaacgacatgcttgccgacatgggcaagtatggtttcgatgcctgtgctcaaacctccgccaaggacttcctggctgacgccaaaacgggcggcctgcaaccgagcatggcgcacaacatggccaccacgctggccgtgcagggcgcgttcttcgatgtggtgaccaactacatcaacgaccccaaggccgacccggccgatgcggcgaagaagctggcggcggctatcaaggctgcccagtaacggctgttcgcgggcacgcccgctcccagatgggccctgctcaccactgttcctggtcatgacgccggacctgtgggagcgggcttgcccgcgaagcggtgcgtagcaccgccactacctcgggatccaatacatgttccagcaagaagttaccattaccgctccgaacggtctgcacacccgccctgctgcccagtttgtaaaagaagctaagggcttcacttctgaaattactgtgacttccaacggcaaaagcgccagcgcgaaaagcctgtttaaactgcagactctgggcctgactcaaggtaccgttgtgactatctccgcagaaggcgaagacgagcagaaagcggttgaacatctggttaaactgatggcggaactcgagtaatttcccgggttcttttaaaaatcagtcacaagtaaggtagggttatgatttcaggcattttagcatccccgggtatcgctttcggtaaagctctgcttctgaaagaagacgaaattgtcattgaccggaaaaaaatttctgccgaccaggttgatcaggaagttgaacgttttctgagcggtcgtgccaaggcatcagcccagctggaaacgatcaaaacgaaagctggtgaaacgttcggtgaagaaaaagaagccatctttgaagggcatattatgctgctcgaagatgaggagctggagcaggaaatcatagccctgattaaagataagcacatgacagctgacgcagctgctcatgaagttatcgaaggtcaggcttctgccctggaagagctggatgatgaatacctgaaagaacgtgcggctgacgtacgtgatatcggtaagcgcctgctgcgcaacatcctgggcctgaagattatcgacctgagcgccattcaggatgaagtcattctggttgccgctgacctgacgccgtccgaaaccgcacagctgaacctgaagaaggtgctgggtttcatcaccgacgcgggtggccgtacttcccacacctctatcatggcgcgttctctggaactacctgctatcgtgggtaccggtagcgtcacctctcaggtgaaaaatgacgactatctgattctggatgccgtaaataatcaggtttacgtcaatccaaccaacgaagttattgataaaatgcgcgctgttcaggagcaagtggcttctgaaaaagcagagcttgctaaactgaaagatctgccagctattacgctggacggtcaccaggtagaagtatgcgctaacattggtacggttcgtgacgttgaaggtgcagagcgtaacggcgctgaaggcgttggtctgtatcgtactgagttcctgttcatggaccgcgacgcactgcccactgaagaagaacagtttgctgcttacaaagcagtggctgaagcgtgtggctcgcaagcggttatcgttcgtaccatggacatcggcggcgacaaagagctgccatacatgaacttcccgaaagaagagaacccgttcctcggctggcgcgctatccgtatcgcgatggatcgtagagagatcctgcgcgatcagctccgcgctatcctgcgtgcctcggctttcggtaaattgcgcattatgttcccgatgatcatctctgttgaagaagtgcgtgcactgcgcaaagagatcgaaatctacaaacaggaactgcgcgacgaaggtaaagcgtttgacgagtcaattgaaatcggcgtaatggtggaaacaccggctgccgcaacaattgcacgtcatttagccaaagaagttgatttctttagtatcggcaccaatgatttaacgcagtacactctggcagttgaccgtggtaatgatatgatttcacacctttaccagccaatgtcaccgtccgtgctgaacttgatcaagcaagttattgatgcttctcatgctgaaggcaaatggactggcatgtgtggtgagcttgctggcgatgaacgtgctacacttctgttgctggggatgggtctggacgaattctctatgagcgccatttctatcccgcgcattaagaagattatccgtaacacgaacttcgaagatgcgaaggtgttagcagagcaggctcttgctcaaccgacaacggacgagttaatgacgctggttaacaagttcattgaagaaaaaacaatctgctaatccacgagatgcggcccaatttactgcttaggagaagatcatgggtttgttcgataaactgaaatctctggtttccgacgacaagaaggataccggaactattgagatcattgctccgctctctggcgagatcgtcaatatcgaagacgtgccggatgtcgtttttgcggaaaaaatcgttggtgatggtattgctatcaaaccaacgggtaacaaaatggtcgcgccagtagacggcaccattggtaaaatctttgaaaccaaccacgcattctctatcgaatctgatagcggcgttgaactgttcgtccacttcggtatcgacaccgttgaactgaaaggcgaaggcttcaagcgtattgctgaagaaggtcagcgcgtgaaagttggcgatactgtcattgaatttgatctgccgctgctggaagagaaagccaagtctaccctgactccggttgttatctccaacatggacgaaatcaaagaactgatcaaactgtccggtagcgtaaccgtgggtgaaaccccggttatccgcatcaagaagtaaactcaggagcactctcaattatgtttaagaatgcatttgctaacctgcaaaaggtcggtaaatcgctgatgctgccggtatccgtactgcctatcgcaggtattctgctgggcgtcggttccgcgaatttcagctggctgcccgccgttgtatcgcatgttatggcagaagcaggcggttccgtctttgcaaacatgccactgatttttgcgatcggtgtcgccctcggctttaccaataacgatggcgtatccgcgctggccgcagttgttgcctatggcatcatggttaaaaccatggccgtggttgcgccactggtactgcatttacctgctgaagaaatcgcctctaaacacctggcggatactggcgtactcggagggattatctccggtgcgatcgcagcgtacatgtttaaccgtttctaccgtattaagctgcctgagtatcttggcttctttgccggtaaacgctttgtgccgatcatttctggcctggctgccatctttactggcgttgtgctgtccttcatttggccgccgattggttctgcaatccagaccttctctcagtgggctgcttaccagaacccggtagttgcgtttggcatttacggtttcatcgaacgttgcctggtaccgtttggtctgcaccacatctggaacgtacctttccagatgcagattggtgaatacaccaacgcagcaggtcaggttttccacggcgacattccgcgttatatggcgggtgacccgactgcgggtaaactgtctggtggcttcctgttcaaaatgtacggtctgccagctgccgcaattgctatctggcactctgctaaaccagaaaaccgcgcgaaagtgggcggtattatgatctccgcggcgctgacctcgttcctgaccggtatcaccgagccgatcgagttctccttcatgttcgttgcgccgatcctgtacatcatccacgcgattctggcaggcctggcattcccaatctgtattcttctggggatgcgtgacggtacgtcgttctcgcacggtctgatcgacttcatcgttctgtctggtaacagcagcaaactgtggctgttcccgatcgtcggtatcggttatgcgattgtttactacaccatcttccgcgtgctgattaaagcactggatctgaaaacgccgggtcgtgaagacgcgactgaagatgcaaaagcgacaggtaccagcgaaatggcaccggctctggttgctgcatttggtggtaaagaaaacattactaacctcgacgcatgtattacccgtctgcgcgtcagcgttgctgatgtgtctaaagtggatcaggccggcctgaagaaactgggcgcagcgggcgtagtggttgctggttctggtgttcaggcgattttcggtactaaatccgataacctgaaaaccgagatggatgagtacatccgtaaccactaactcgtctacaccatcaataagaaaaagaggacgcaaagggatggaacagcgcaaacgcatcaagacactgggatcgttggccttgcttgcacttgtaggcagcagcggtacacaggctgccgaggctttttccagcgaatccaaatggatgaccggcgactggggcggcacccggaccgagctgctggacaagggctatgacttcaccctcgattatgtgggtgaggtggctggcaacctgcatggcggctacaacgacgacaagacggcacgctacagcgaccagttcgccctcggcgcgcatctggacttgcagaagatactgggctggcatgatgccgagttcaagctggcaatcaccgagcgaagcggtcgcaacctgtccaacgaccgcatcagcgacccgcgcgccgggcagttcagctcggtgcaggaggtgtggggccgtggccagacctggcgcctgacccagatgtggatcaagcagaagtacttcgacggcgcgctggacgtgaaatttggccgttttggcgagggcgaggacttcaacagcttcccttgcgacttccagaacctggccttctgcggctcgcaggtgggcaactgggtgggcggcatctggtacaactggccggtcagccagtgggcgctgcgggtgaagtacaacatcacgcctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctttccttcttcactgtcccttctagaacgttctagaagggacagtgaagaaggaacacccgctcgcgggtgggcctactctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggtcaggcgtccttttgcttggtgccgaagatcttgtcaccggcatcacccaggcctggcacgatgtagccgtgctcgttcaggcgctggtcgatcgaggcggtgtagatcttcacgtccgggtgggctttttccaccacctcgatgccttctggtgcggcgaccagcaccatggcgcgaatctctttgcagccggcctttttcagcaggtcgatggtggcaaccatcgagccgccggtggccagcatcgggtcgatgatcagggccaggcgctggttgatgtccggcgcgagcttttccagataggtgtgggcttcgagggtttcttcgtttcgggcaacgccgacggcgctgaccttggcccccgggatcaggctgagcacgccgtcgagcatgccgatgccggcgcgcaggatcggtactacggtgatcttcttgccggcgattttttcaaccgagaccttgccacaccagccgtcgatctcgtaggtttcgaggggcaggtcctgggtggcttcatacgtcaggagcgcgccgacttcctgggcgagttcgcgaaaattcttggtgctgatatcggcacggcgcatcaggccaagcttgtggcggatcagcggatggcggatctcacgagtgggcataggggagggctccgaaaggcgggcaaaaaaaccgcgctagattaatctattcagcctgtgctgtcgtctggtcattctggacgttagtccataaatgcttgatctgtgacgagcggatgcgtacctttgcccgcttttccaaaatgctagccggctacgtatcgataagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctg

SEQ ID NO: 39 - DNA, 7236 bp, Artificial Sequence
  knockout construct
gcatcgcttcgttacctgacgaggcgggcacgctgtttagacttcatgctacgcaagcgatcgacttgcacggcataacaacaagaaaggtgctccgatgaattccacgcttcgtctcgctgccgcaatttcctttgcctcgttaatccccttgggtgcccaggctgctgacgccaaaggcagtgtcgaagtggtgcactggtggacctccggtggtgaaaaagcggcggtcgatgtgctcaaggcccaggtcgaaaaagacggcttcatctggaaggacggcgccgtcgccggcggcggtggtgcaacggccatgaccgtgctcaaaagccgcgcagtggccggcaacccgccgggcgtcgcgcagatcaaaggcccggacatccaggactgggcggccaccggcctgctcgacgccgatgtgctcaaggatgtggccaaggaaggaaagtgggactcgctgctcgacaagaaagtggccgacaccgtgaagtacgacggtgactacgttgccgtaccggtgaatatccaccgcatcaactggctgtggatcaaccccgaggtgttcaaaaaagccggcatcgacaaggcgcccaccaccctcgacgaattctacgccgccgccgacaagctcaaggctgccggtttcatcccgctcgcccatggtgggcaaccctggcaggacagcaccgtgttcgaaagcgtggtgctgtcggtgatgggcgtcgatggctacaagaaggccttggtcgacctcgacagcgcaacgctgaccgggccgcagatggtcaaggcgctgaccgagttgaagaaagtcgccacctacatggacccggacggcaagggccaggactggaacctggaagccgccaaggtcattaacggcaaggccggcatgcagatcatgggcgactgggccaagagcgaatggaccctggcgaagaaaactgctggcaaggattaccagtgcgtgccgttccccggtactgacaagtccttcctgtacaacatcgactcgttggtggtgttcaagcagaacaacgctggcacctctgctggtcagcaggacatcgcccgcaaggtgttgggtgaggacttccagaaggtcttcagcatcaacaagggttcgatcccggtacgcaacgacatgcttgccgacatgggcaagtatggtttcgatgcctgtgctcaaacctccgccaaggacttcctggctgacgccaaaacgggcggcctgcaaccgagcatggcgcacaacatggccaccacgctggccgtgcagggcgcgttcttcgatgtggtgaccaactacatcaacgaccccaaggccgacccggccgatgcggcgaagaagctggcggcggctatcaaggctgcccagtaacggctgttcgcgggcacgcccgctcccagatgggccctgctcaccactgttcctggtcatgacgccggacctgtgggagcgggcttgcccgcgaagcggtgcgtagcaccgccactacctcgggatccaatacatgttccagcaagaagttaccattaccgctccgaacggtctgcacacccgccctgctgcccagtttgtaaaagaagctaagggcttcacttctgaaattactgtgacttccaacggcaaaagcgccagcgcgaaaagcctgtttaaactgcagactctgggcctgactcaaggtaccgttgtgactatctccgcagaaggcgaagacgagcagaaagcggttgaacatctggttaaactgatggcggaactcgagtaatttcccgggttcttttaaaaatcagtcacaagtaaggtagggttatgatttcaggcattttagcatccccgggtatcgctttcggtaaagctctgcttctgaaagaagacgaaattgtcattgaccggaaaaaaatttctgccgaccaggttgatcaggaagttgaacgttttctgagcggtcgtgccaaggcatcagcccagctggaaacgatcaaaacgaaagctggtgaaacgttcggtgaagaaaaagaagccatctttgaagggcatattatgctgctcgaagatgaggagctggagcaggaaatcatagccctgattaaagataagcacatgacagctgacgcagctgctcatgaagttatcgaaggtcaggcttctgccctggaagagctggatgatgaatacctgaaagaacgtgcggctgacgtacgtgatatcggtaagcgcctgctgcgcaacatcctgggcctgaagattatcgacctgagcgccattcaggatgaagtcattctggttgccgctgacctgacgccgtccgaaaccgcacagctgaacctgaagaaggtgctgggtttcatcaccgacgcgggtggccgtacttcccacacctctatcatggcgcgttctctggaactacctgctatcgtgggtaccggtagcgtcacctctcaggtgaaaaatgacgactatctgattctggatgccgtaaataatcaggtttacgtcaatccaaccaacgaagttattgataaaatgcgcgctgttcaggagcaagtggcttctgaaaaagcagagcttgctaaactgaaagatctgccagctattacgctggacggtcaccaggtagaagtatgcgctaacattggtacggttcgtgacgttgaaggtgcagagcgtaacggcgctgaaggcgttggtctgtatcgtactgagttcctgttcatggaccgcgacgcactgcccactgaagaagaacagtttgctgcttacaaagcagtggctgaagcgtgtggctcgcaagcggttatcgttcgtaccatggacatcggcggcgacaaagagctgccatacatgaacttcccgaaagaagagaacccgttcctcggctggcgcgctatccgtatcgcgatggatcgtagagagatcctgcgcgatcagctccgcgctatcctgcgtgcctcggctttcggtaaattgcgcattatgttcccgatgatcatctctgttgaagaagtgcgtgcactgcgcaaagagatcgaaatctacaaacaggaactgcgcgacgaaggtaaagcgtttgacgagtcaattgaaatcggcgtaatggtggaaacaccggctgccgcaacaattgcacgtcatttagccaaagaagttgatttctttagtatcggcaccaatgatttaacgcagtacactctggcagttgaccgtggtaatgatatgatttcacacctttaccagccaatgtcaccgtccgtgctgaacttgatcaagcaagttattgatgcttctcatgctgaaggcaaatggactggcatgtgtggtgagcttgctggcgatgaacgtgctacacttctgttgctggggatgggtctggacgaattctctatgagcgccatttctatcccgcgcattaagaagattatccgtaacacgaacttcgaagatgcgaaggtgttagcagagcaggctcttgctcaaccgacaacggacgagttaatgacgctggttaacaagttcattgaagaaaaaacaatctgctaatccacgagatgcggcccaatttactgcttaggagaagatcatgggtttgttcgataaactgaaatctctggtttccgacgacaagaaggataccggaactattgagatcattgctccgctctctggcgagatcgtcaatatcgaagacgtgccggatgtcgtttttgcggaaaaaatcgttggtgatggtattgctatcaaaccaacgggtaacaaaatggtcgcgccagtagacggcaccattggtaaaatctttgaaaccaaccacgcattctctatcgaatctgatagcggcgttgaactgttcgtccacttcggtatcgacaccgttgaactgaaaggcgaaggcttcaagcgtattgctgaagaaggtcagcgcgtgaaagttggcgatactgtcattgaatttgatctgccgctgctggaagagaaagccaagtctaccctgactccggttgttatctccaacatggacgaaatcaaagaactgatcaaactgtccggtagcgtaaccgtgggtgaaaccccggttatccgcatcaagaagtaaactcaggagcactctcaattatgtttaagaatgcatttgctaacctgcaaaaggtcggtaaatcgctgatgctgccggtatccgtactgcctatcgcaggtattctgctgggcgtcggttccgcgaatttcagctggctgcccgccgttgtatcgcatgttatggcagaagcaggcggttccgtctttgcaaacatgccactgatttttgcgatcggtgtcgccctcggctttaccaataacgatggcgtatccgcgctggccgcagttgttgcctatggcatcatggttaaaaccatggccgtggttgcgccactggtactgcatttacctgctgaagaaatcgcctctaaacacctggcggatactggcgtactcggagggattatctccggtgcgatcgcagcgtacatgtttaaccgtttctaccgtattaagctgcctgagtatcttggcttctttgccggtaaacgctttgtgccgatcatttctggcctggctgccatctttactggcgttgtgctgtccttcatttggccgccgattggttctgcaatccagaccttctctcagtgggctgcttaccagaacccggtagttgcgtttggcatttacggtttcatcgaacgttgcctggtaccgtttggtctgcaccacatctggaacgtacctttccagatgcagattggtgaatacaccaacgcagcaggtcaggttttccacggcgacattccgcgttatatggcgggtgacccgactgcgggtaaactgtctggtggcttcctgttcaaaatgtacggtctgccagctgccgcaattgctatctggcactctgctaaaccagaaaaccgcgcgaaagtgggcggtattatgatctccgcggcgctgacctcgttcctgaccggtatcaccgagccgatcgagttctccttcatgttcgttgcgccgatcctgtacatcatccacgcgattctggcaggcctggcattcccaatctgtattcttctggggatgcgtgacggtacgtcgttctcgcacggtctgatcgacttcatcgttctgtctggtaacagcagcaaactgtggctgttcccgatcgtcggtatcggttatgcgattgtttactacaccatcttccgcgtgctgattaaagcactggatctgaaaacgccgggtcgtgaagacgcgactgaagatgcaaaagcgacaggtaccagcgaaatggcaccggctctggttgctgcatttggtggtaaagaaaacattactaacctcgacgcatgtattacccgtctgcgcgtcagcgttgctgatgtgtctaaagtggatcaggccggcctgaagaaactgggcgcagcgggcgtagtggttgctggttctggtgttcaggcgattttcggtactaaatccgataacctgaaaaccgagatggatgagtacatccgtaaccactaactcgtctacaccatcaataagaaaaagaggacgcaaagggatggaacagcgcaaacgcatcaagacactgggatcgttggccttgcttgcacttgtaggcagcagcggtacacaggctgccgaggctttttccagcgaatccaaatggatgaccggcgactggggcggcacccggaccgagctgctggacaagggctatgacttcaccctcgattatgtgggtgaggtggctggcaacctgcatggcggctacaacgacgacaagacggcacgctacagcgaccagttcgccctcggcgcgcatctggacttgcagaagatactgggctggcatgatgccgagttcaagctggcaatcaccgagcgaagcggtcgcaacctgtccaacgaccgcatcagcgacccgcgcgccgggcagttcagctcggtgcaggaggtgtggggccgtggccagacctggcgcctgacccagatgtggatcaagcagaagtacttcgacggcgcgctggacgtgaaatttggccgttttggcgagggcgaggacttcaacagcttcccttgcgacttccagaacctggccttctgcggctcgcaggtgggcaactgggtgggcggcatctggtacaactggccggtcagccagtgggcgctgcgggtgaagtacaacatcacgccggagttcttcgtacaggtcggggccttcgagcagaacccttccaacctggaaaccggcaacggcttcaagctcagcggcagtggtaccaagggggcgatcttgccggtggaagcggtgtggtcgcccaaggtcaatggcctgccgggcgagtaccgcctgggttactactacagcacggccaaggctgacgatgtgtacgacgacgtcaacggcaacccgcaggcgctgacaggtgaagccttcaagtcgcactccagcaagcacggatggtgggtggtggcgcagcagcaggtcactgcccatggcggcgacgtcaaccggggcctcagcctgttcgccaacttcaccgtgcacgacaaggccaccaacgtggtcgacaactaccagcaggtggggctggtctacaaaggcgctttcgacgcccggcccaaggatgacatcggcttcggcgtggcgcgtattcatgtgaatgacgacgtgaagaagcgcgccgaactgctcaacgcacagagcggcatcaacgattacgacaaccctggtttcgtgccgctgcagcgtaccgaatacaacgcagagctctactacggcttccacgttaccaactggctgaccgtgaggcccaacctgcagtacatcaagagccctggcggggtggacgaggtggataacgcgctggtcgctggcttgaagattcagtcgtcattctgaggcaacttgttgtaaatttacgccaatccattttcggttcccggcacccactggtctgcagtgggtgttggggataggccgagacagcgctatctcaaacaacaagagctctgaaccatgcccgaacatccgctccatcgcttcttctcctcgcagcggccccggccgacattcgagtgggagcgttatcagcagcgcgatgtcctgatcatcgaccacccccgttgccaggcggtgttcagccgccagggcgcgcagttgctgcactttcaaccagcgggtgaacggccgtggctgtggt

SEQ ID NO: 40 - DNA, 38 bp, Artificial Sequence
  primer
tacctcgggatccaatacatggcaacttccatcccaac

SEQ ID NO: 41 - DNA, 21 bp, Artificial Sequence
  primer
tcagcttttgctgccgatgag

SEQ ID NO: 42 - DNA, 47 bp, Artificial Sequence
  primer
tcatcggcagcaaaagctgactcgtctacaccatcaataagaaaaag

SEQ ID NO: 43 - DNA, 1257 bp, Artificial Sequence
  PCR product
tacctcgggatccaatacatggcaacttccatcccaacgaataatccgctgcacactgaaacctcctcccagaaaaattacggtttcgcgctcaccagcctcaccttgctgtttttcatgtggggcttcatcacctgtctcaacgacatcctgatcccgcatctaaagaacgttttccagctcaactatacccagtcgatgctgatccagttctgcttcttcggggcctatttcatcgtttccctgcccgcagggcagcttgtaaagcgcatttcctataagcgtggcatcgtcgtcggcctcatcgtggcggcaatcggctgcgcgcttttcatcccggccgcgtcataccgcgtctatgcgctgttcctcggcgcgctcttcgtgcttgcgtccggcgttacgatccttcaggttgccgccaatccctatgtgacgatccttggcaagcctgaaaccgccgcaagccgcctgacactgacacaggccttcaactcgctcggcaccacggttgcgccggtctttggcgcagtcctgatcctttcagcggcaacggacgccaccgtcaatgccgaagccgacgcggttcgcttcccatatcttcttctggcgcttgccttcacggttcttgccatcatctttgcaatcctgaaaccgccggatgtgcaggaagacgaacccgccctttccgacaagaaagagggcagcgcctggcaatatcgccacctggttctgggagcgatcggcattttcgtgtacgtcggcgctgaggtcagcgtcggcagcttcctcgtgaatttcctgagcgatcccaccgttgctggcctctccgaaaccgacgccgcccatcacgtggcctatttctggggcggggccatggtgggccgcttcatcggctcggcagccatgcgttatatcgatgacggcaaggcccttgccttcaacgcatttgtcgccatcatcctgctgttcatcaccgttgccaccacgggccatatcgccatgtggtcggtgctggccatcgggctgttcaattcgatcatgttccccacgatcttcagcctcgccttgcatggccttggcagccacaccagccagggttccggcattttgtgcctggccatcgtcggcggcgcgattgttccgctgatccagggcgcattggctgatgcgatcggcattcatcttgccttcctgatgccgatcatctgctatgcctatatcgccttctatggcctcatcggcagcaaaagctga

SEQ ID NO: 44 - DNA, 710 bp, Artificial Sequence
  PCR procudt
tcatcggcagcaaaagctgactcgtctacaccatcaataagaaaaagaggacgcaaagggatggaacagcgcaaacgcatcaagacactgggatcgttggccttgcttgcacttgtaggcagcagcggtacacaggctgccgaggctttttccagcgaatccaaatggatgaccggcgactggggcggcacccggaccgagctgctggacaagggctatgacttcaccctcgattatgtgggtgaggtggctggcaacctgcatggcggctacaacgacgacaagacggcacgctacagcgaccagttcgccctcggcgcgcatctggacttgcagaagatactgggctggcatgatgccgagttcaagctggcaatcaccgagcgaagcggtcgcaacctgtccaacgaccgcatcagcgacccgcgcgccgggcagttcagctcggtgcaggaggtgtggggccgtggccagacctggcgcctgacccagatgtggatcaagcagaagtacttcgacggcgcgctggacgtgaaatttggccgttttggcgagggcgaggacttcaacagcttcccttgcgacttccagaacctggccttctgcggctcgcaggtgggcaactgggtgggcggcatctggtacaactggccggtcagccagtgggcgctgcgggtgaagtacaacatcacgcctgcaggcatgcaagc

SEQ ID NO: 45 - DNA, 7379 bp, Artificial Sequence
  vector
atggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgggctcgatcccctcgcgagttggttcagctgctgcctgaggctggacgacctcgcggagttctaccggcagtgcaaatccgtcggcatccaggaaaccagcagcggctatccgcgcatccatgcccccgaactgcaggagtggggaggcacgatggccgctttggtcccggccgccaaggtcattaacggcaaggccggcatgcagatcatgggcgactgggccaagagcgaatggaccctggcgaagaaaactgctggcaaggattaccagtgcgtgccgttccccggtactgacaagtccttcctgtacaacatcgactcgttggtggtgttcaagcagaacaacgctggcacctctgctggtcagcaggacatcgcccgcaaggtgttgggtgaggacttccagaaggtcttcagcatcaacaagggttcgatcccggtacgcaacgacatgcttgccgacatgggcaagtatggtttcgatgcctgtgctcaaacctccgccaaggacttcctggctgacgccaaaacgggcggcctgcaaccgagcatggcgcacaacatggccaccacgctggccgtgcagggcgcgttcttcgatgtggtgaccaactacatcaacgaccccaaggccgacccggccgatgcggcgaagaagctggcggcggctatcaaggctgcccagtaacggctgttcgcgggcacgcccgctcccagatgggccctgctcaccactgttcctggtcatgacgccggacctgtgggagcgggcttgcccgcgaagcggtgcgtagcaccgccactacctcgggatccaatacatggcaacttccatcccaacgaataatccgctgcacactgaaacctcctcccagaaaaattacggtttcgcgctcaccagcctcaccttgctgtttttcatgtggggcttcatcacctgtctcaacgacatcctgatcccgcatctaaagaacgttttccagctcaactatacccagtcgatgctgatccagttctgcttcttcggggcctatttcatcgtttccctgcccgcagggcagcttgtaaagcgcatttcctataagcgtggcatcgtcgtcggcctcatcgtggcggcaatcggctgcgcgcttttcatcccggccgcgtcataccgcgtctatgcgctgttcctcggcgcgctcttcgtgcttgcgtccggcgttacgatccttcaggttgccgccaatccctatgtgacgatccttggcaagcctgaaaccgccgcaagccgcctgacactgacacaggccttcaactcgctcggcaccacggttgcgccggtctttggcgcagtcctgatcctttcagcggcaacggacgccaccgtcaatgccgaagccgacgcggttcgcttcccatatcttcttctggcgcttgccttcacggttcttgccatcatctttgcaatcctgaaaccgccggatgtgcaggaagacgaacccgccctttccgacaagaaagagggcagcgcctggcaatatcgccacctggttctgggagcgatcggcattttcgtgtacgtcggcgctgaggtcagcgtcggcagcttcctcgtgaatttcctgagcgatcccaccgttgctggcctctccgaaaccgacgccgcccatcacgtggcctatttctggggcggggccatggtgggccgcttcatcggctcggcagccatgcgttatatcgatgacggcaaggcccttgccttcaacgcatttgtcgccatcatcctgctgttcatcaccgttgccaccacgggccatatcgccatgtggtcggtgctggccatcgggctgttcaattcgatcatgttccccacgatcttcagcctcgccttgcatggccttggcagccacaccagccagggttccggcattttgtgcctggccatcgtcggcggcgcgattgttccgctgatccagggcgcattggctgatgcgatcggcattcatcttgccttcctgatgccgatcatctgctatgcctatatcgccttctatggcctcatcggcagcaaaagctgactcgtctacaccatcaataagaaaaagaggacgcaaagggatggaacagcgcaaacgcatcaagacactgggatcgttggccttgcttgcacttgtaggcagcagcggtacacaggctgccgaggctttttccagcgaatccaaatggatgaccggcgactggggcggcacccggaccgagctgctggacaagggctatgacttcaccctcgattatgtgggtgaggtggctggcaacctgcatggcggctacaacgacgacaagacggcacgctacagcgaccagttcgccctcggcgcgcatctggacttgcagaagatactgggctggcatgatgccgagttcaagctggcaatcaccgagcgaagcggtcgcaacctgtccaacgaccgcatcagcgacccgcgcgccgggcagttcagctcggtgcaggaggtgtggggccgtggccagacctggcgcctgacccagatgtggatcaagcagaagtacttcgacggcgcgctggacgtgaaatttggccgttttggcgagggcgaggacttcaacagcttcccttgcgacttccagaacctggccttctgcggctcgcaggtgggcaactgggtgggcggcatctggtacaactggccggtcagccagtgggcgctgcgggtgaagtacaacatcacgcctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctttccttcttcactgtcccttctagaacgttctagaagggacagtgaagaaggaacacccgctcgcgggtgggcctactctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggtcaggcgtccttttgcttggtgccgaagatcttgtcaccggcatcacccaggcctggcacgatgtagccgtgctcgttcaggcgctggtcgatcgaggcggtgtagatcttcacgtccgggtgggctttttccaccacctcgatgccttctggtgcggcgaccagcaccatggcgcgaatctctttgcagccggcctttttcagcaggtcgatggtggcaaccatcgagccgccggtggccagcatcgggtcgatgatcagggccaggcgctggttgatgtccggcgcgagcttttccagataggtgtgggcttcgagggtttcttcgtttcgggcaacgccgacggcgctgaccttggcccccgggatcaggctgagcacgccgtcgagcatgccgatgccggcgcgcaggatcggtactacggtgatcttcttgccggcgattttttcaaccgagaccttgccacaccagccgtcgatctcgtaggtttcgaggggcaggtcctgggtggcttcatacgtcaggagcgcgccgacttcctgggcgagttcgcgaaaattcttggtgctgatatcggcacggcgcatcaggccaagcttgtggcggatcagcggatggcggatctcacgagtgggcataggggagggctccgaaaggcgggcaaaaaaaccgcgctagattaatctattcagcctgtgctgtcgtctggtcattctggacgttagtccataaatgcttgatctgtgacgagcggatgcgtacctttgcccgcttttccaaaatgctagccggctacgtatcgataagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctg

SEQ ID NO: 46 - DNA, 4441 bp, Artificial Sequence
  knockout construct
gcatcgcttcgttacctgacgaggcgggcacgctgtttagacttcatgctacgcaagcgatcgacttgcacggcataacaacaagaaaggtgctccgatgaattccacgcttcgtctcgctgccgcaatttcctttgcctcgttaatccccttgggtgcccaggctgctgacgccaaaggcagtgtcgaagtggtgcactggtggacctccggtggtgaaaaagcggcggtcgatgtgctcaaggcccaggtcgaaaaagacggcttcatctggaaggacggcgccgtcgccggcggcggtggtgcaacggccatgaccgtgctcaaaagccgcgcagtggccggcaacccgccgggcgtcgcgcagatcaaaggcccggacatccaggactgggcggccaccggcctgctcgacgccgatgtgctcaaggatgtggccaaggaaggaaagtgggactcgctgctcgacaagaaagtggccgacaccgtgaagtacgacggtgactacgttgccgtaccggtgaatatccaccgcatcaactggctgtggatcaaccccgaggtgttcaaaaaagccggcatcgacaaggcgcccaccaccctcgacgaattctacgccgccgccgacaagctcaaggctgccggtttcatcccgctcgcccatggtgggcaaccctggcaggacagcaccgtgttcgaaagcgtggtgctgtcggtgatgggcgtcgatggctacaagaaggccttggtcgacctcgacagcgcaacgctgaccgggccgcagatggtcaaggcgctgaccgagttgaagaaagtcgccacctacatggacccggacggcaagggccaggactggaacctggaagccgccaaggtcattaacggcaaggccggcatgcagatcatgggcgactgggccaagagcgaatggaccctggcgaagaaaactgctggcaaggattaccagtgcgtgccgttccccggtactgacaagtccttcctgtacaacatcgactcgttggtggtgttcaagcagaacaacgctggcacctctgctggtcagcaggacatcgcccgcaaggtgttgggtgaggacttccagaaggtcttcagcatcaacaagggttcgatcccggtacgcaacgacatgcttgccgacatgggcaagtatggtttcgatgcctgtgctcaaacctccgccaaggacttcctggctgacgccaaaacgggcggcctgcaaccgagcatggcgcacaacatggccaccacgctggccgtgcagggcgcgttcttcgatgtggtgaccaactacatcaacgaccccaaggccgacccggccgatgcggcgaagaagctggcggcggctatcaaggctgcccagtaacggctgttcgcgggcacgcccgctcccagatgggccctgctcaccactgttcctggtcatgacgccggacctgtgggagcgggcttgcccgcgaagcggtgcgtagcaccgccactacctcgggatccaatacatggcaacttccatcccaacgaataatccgctgcacactgaaacctcctcccagaaaaattacggtttcgcgctcaccagcctcaccttgctgtttttcatgtggggcttcatcacctgtctcaacgacatcctgatcccgcatctaaagaacgttttccagctcaactatacccagtcgatgctgatccagttctgcttcttcggggcctatttcatcgtttccctgcccgcagggcagcttgtaaagcgcatttcctataagcgtggcatcgtcgtcggcctcatcgtggcggcaatcggctgcgcgcttttcatcccggccgcgtcataccgcgtctatgcgctgttcctcggcgcgctcttcgtgcttgcgtccggcgttacgatccttcaggttgccgccaatccctatgtgacgatccttggcaagcctgaaaccgccgcaagccgcctgacactgacacaggccttcaactcgctcggcaccacggttgcgccggtctttggcgcagtcctgatcctttcagcggcaacggacgccaccgtcaatgccgaagccgacgcggttcgcttcccatatcttcttctggcgcttgccttcacggttcttgccatcatctttgcaatcctgaaaccgccggatgtgcaggaagacgaacccgccctttccgacaagaaagagggcagcgcctggcaatatcgccacctggttctgggagcgatcggcattttcgtgtacgtcggcgctgaggtcagcgtcggcagcttcctcgtgaatttcctgagcgatcccaccgttgctggcctctccgaaaccgacgccgcccatcacgtggcctatttctggggcggggccatggtgggccgcttcatcggctcggcagccatgcgttatatcgatgacggcaaggcccttgccttcaacgcatttgtcgccatcatcctgctgttcatcaccgttgccaccacgggccatatcgccatgtggtcggtgctggccatcgggctgttcaattcgatcatgttccccacgatcttcagcctcgccttgcatggccttggcagccacaccagccagggttccggcattttgtgcctggccatcgtcggcggcgcgattgttccgctgatccagggcgcattggctgatgcgatcggcattcatcttgccttcctgatgccgatcatctgctatgcctatatcgccttctatggcctcatcggcagcaaaagctgactcgtctacaccatcaataagaaaaagaggacgcaaagggatggaacagcgcaaacgcatcaagacactgggatcgttggccttgcttgcacttgtaggcagcagcggtacacaggctgccgaggctttttccagcgaatccaaatggatgaccggcgactggggcggcacccggaccgagctgctggacaagggctatgacttcaccctcgattatgtgggtgaggtggctggcaacctgcatggcggctacaacgacgacaagacggcacgctacagcgaccagttcgccctcggcgcgcatctggacttgcagaagatactgggctggcatgatgccgagttcaagctggcaatcaccgagcgaagcggtcgcaacctgtccaacgaccgcatcagcgacccgcgcgccgggcagttcagctcggtgcaggaggtgtggggccgtggccagacctggcgcctgacccagatgtggatcaagcagaagtacttcgacggcgcgctggacgtgaaatttggccgttttggcgagggcgaggacttcaacagcttcccttgcgacttccagaacctggccttctgcggctcgcaggtgggcaactgggtgggcggcatctggtacaactggccggtcagccagtgggcgctgcgggtgaagtacaacatcacgccggagttcttcgtacaggtcggggccttcgagcagaacccttccaacctggaaaccggcaacggcttcaagctcagcggcagtggtaccaagggggcgatcttgccggtggaagcggtgtggtcgcccaaggtcaatggcctgccgggcgagtaccgcctgggttactactacagcacggccaaggctgacgatgtgtacgacgacgtcaacggcaacccgcaggcgctgacaggtgaagccttcaagtcgcactccagcaagcacggatggtgggtggtggcgcagcagcaggtcactgcccatggcggcgacgtcaaccggggcctcagcctgttcgccaacttcaccgtgcacgacaaggccaccaacgtggtcgacaactaccagcaggtggggctggtctacaaaggcgctttcgacgcccggcccaaggatgacatcggcttcggcgtggcgcgtattcatgtgaatgacgacgtgaagaagcgcgccgaactgctcaacgcacagagcggcatcaacgattacgacaaccctggtttcgtgccgctgcagcgtaccgaatacaacgcagagctctactacggcttccacgttaccaactggctgaccgtgaggcccaacctgcagtacatcaagagccctggcggggtggacgaggtggataacgcgctggtcgctggcttgaagattcagtcgtcattctgaggcaacttgttgtaaatttacgccaatccattttcggttcccggcacccactggtctgcagtgggtgttggggataggccgagacagcgctatctcaaacaacaagagctctgaaccatgcccgaacatccgctccatcgcttcttctcctcgcagcggccccggccgacattcgagtgggagcgttatcagcagcgcgatgtcctgatcatcgaccacccccgttgccaggcggtgttcagccgccagggcgcgcagttgctgcactttcaaccagcgggtgaacggccgtggctgtggt

SEQ ID NO: 47 - DNA, 47 bp, Artificial Sequence
  primer
tacctcgggatccaatacatggcaagcacctttatccaggccgacag

SEQ ID NO: 48 - DNA, 25 bp, Artificial Sequence
  primer
tcaatggaccttgcccttgcgaatg

SEQ ID NO: 49 - DNA, 47 bp, Artificial Sequence
  primer
gcaagggcaaggtccattgactcgtctacaccatcaataagaaaaag

SEQ ID NO: 50 - DNA, 1494 bp, Artificial Sequence
  PCR product
tacctcgggatccaatacatggcaagcacctttatccaggccgacagcccggagaaaagcaaaaaactgcccccgctgaccgaaggcccgtaccgcaagcgcttgttctatgtggccctggtagctaccttcggcggtctgctcttcggctacgacaccggcgtcatcaatggtgcgctgaaccccatgacgcgcgagctggggctgaccgcattcaccgagggcgtggtgaccagcagcctgctgtttggggccgcggcgggcgccatgttcttcggtcgcatcagcgacaactgggggcgccgcaaaaccatcatctcgctggctgtcgccttcttcgtggggaccatgatctgcgtattcgccccatcgttcgccgtgatggtggtgggccgcgtgttgctgggcctggcggtcggcggcgcgagcacggtggtgcccgtctacttggccgaactggcgccgttcgagatccggggctcgctggccggccgcaacgaactgatgatcgtggtggggcaactggccgcattcgtgattaatgccatcatcggcaatgtgttcggccatcacgacggggtctggcgctacatgctggccatcgcagccatcccggccattgccctgttcttcggcatgctgcgtgtacctgagtccccccgctggctggtggagcgcgggcgtattgacgaagcccgcgcggtcctggagacgatccgtccattggaacgcgcccacgcggaggtggccgacgtcgaacacctggcccgcgaggagcacgctgtgtcggagaagtcgatgggcctgcgcgaaatcctgagctcgaagtggctggtgcgcatcctgctcgtgggcattggtctcggtgtggcgcagcaactcaccggtatcaacagcatcatgtactacggtcaggtcgtcttgatcgaagccggctttagcgagaacgctgcgctgatcgccaacgtggcccccggcgtgattgccgtggtcggcgcctttatcgcgttgtggatgatggatcgtattaaccgtcgtaccaccttgatcaccgggtatagcctgaccaccatctcccacgtactgattgggatcgccagcgtggccttccccgtgggcgatcctctgcgtccgtacgtcatcctgaccctggtcgtcgtgttcgtaggcagcatgcaaaccttcctgaacgtggccacgtgggtgatgctgagcgagctctttccgctggcgatgcgggggttcgccattggcatcagcgtcttctttctctggatcgcgaacgcgttcctgggcctgttttttcctacgatcatggaagccgtgggcctgacgggcacctttttcatgttcgccggtatcggggtcgtagccctgattttcatttacacccaagtgccggaaacccgggggcgcaccctggaagaaatcgacgaggacgtgaccagcggcgtcatcttcaacaaggacattcgcaagggcaaggtccattga

SEQ ID NO: 51 - DNA, 710 bp, Artificial Sequence
  PCR product
gcaagggcaaggtccattgactcgtctacaccatcaataagaaaaagaggacgcaaagggatggaacagcgcaaacgcatcaagacactgggatcgttggccttgcttgcacttgtaggcagcagcggtacacaggctgccgaggctttttccagcgaatccaaatggatgaccggcgactggggcggcacccggaccgagctgctggacaagggctatgacttcaccctcgattatgtgggtgaggtggctggcaacctgcatggcggctacaacgacgacaagacggcacgctacagcgaccagttcgccctcggcgcgcatctggacttgcagaagatactgggctggcatgatgccgagttcaagctggcaatcaccgagcgaagcggtcgcaacctgtccaacgaccgcatcagcgacccgcgcgccgggcagttcagctcggtgcaggaggtgtggggccgtggccagacctggcgcctgacccagatgtggatcaagcagaagtacttcgacggcgcgctggacgtgaaatttggccgttttggcgagggcgaggacttcaacagcttcccttgcgacttccagaacctggccttctgcggctcgcaggtgggcaactgggtgggcggcatctggtacaactggccggtcagccagtgggcgctgcgggtgaagtacaacatcacgcctgcaggcatgcaagc

SEQ ID NO: 52 - DNA, 7616 bp, Artificial Sequence
  vector
atggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgggctcgatcccctcgcgagttggttcagctgctgcctgaggctggacgacctcgcggagttctaccggcagtgcaaatccgtcggcatccaggaaaccagcagcggctatccgcgcatccatgcccccgaactgcaggagtggggaggcacgatggccgctttggtcccggccgccaaggtcattaacggcaaggccggcatgcagatcatgggcgactgggccaagagcgaatggaccctggcgaagaaaactgctggcaaggattaccagtgcgtgccgttccccggtactgacaagtccttcctgtacaacatcgactcgttggtggtgttcaagcagaacaacgctggcacctctgctggtcagcaggacatcgcccgcaaggtgttgggtgaggacttccagaaggtcttcagcatcaacaagggttcgatcccggtacgcaacgacatgcttgccgacatgggcaagtatggtttcgatgcctgtgctcaaacctccgccaaggacttcctggctgacgccaaaacgggcggcctgcaaccgagcatggcgcacaacatggccaccacgctggccgtgcagggcgcgttcttcgatgtggtgaccaactacatcaacgaccccaaggccgacccggccgatgcggcgaagaagctggcggcggctatcaaggctgcccagtaacggctgttcgcgggcacgcccgctcccagatgggccctgctcaccactgttcctggtcatgacgccggacctgtgggagcgggcttgcccgcgaagcggtgcgtagcaccgccactacctcgggatccaatacatggcaagcacctttatccaggccgacagcccggagaaaagcaaaaaactgcccccgctgaccgaaggcccgtaccgcaagcgcttgttctatgtggccctggtagctaccttcggcggtctgctcttcggctacgacaccggcgtcatcaatggtgcgctgaaccccatgacgcgcgagctggggctgaccgcattcaccgagggcgtggtgaccagcagcctgctgtttggggccgcggcgggcgccatgttcttcggtcgcatcagcgacaactgggggcgccgcaaaaccatcatctcgctggctgtcgccttcttcgtggggaccatgatctgcgtattcgccccatcgttcgccgtgatggtggtgggccgcgtgttgctgggcctggcggtcggcggcgcgagcacggtggtgcccgtctacttggccgaactggcgccgttcgagatccggggctcgctggccggccgcaacgaactgatgatcgtggtggggcaactggccgcattcgtgattaatgccatcatcggcaatgtgttcggccatcacgacggggtctggcgctacatgctggccatcgcagccatcccggccattgccctgttcttcggcatgctgcgtgtacctgagtccccccgctggctggtggagcgcgggcgtattgacgaagcccgcgcggtcctggagacgatccgtccattggaacgcgcccacgcggaggtggccgacgtcgaacacctggcccgcgaggagcacgctgtgtcggagaagtcgatgggcctgcgcgaaatcctgagctcgaagtggctggtgcgcatcctgctcgtgggcattggtctcggtgtggcgcagcaactcaccggtatcaacagcatcatgtactacggtcaggtcgtcttgatcgaagccggctttagcgagaacgctgcgctgatcgccaacgtggcccccggcgtgattgccgtggtcggcgcctttatcgcgttgtggatgatggatcgtattaaccgtcgtaccaccttgatcaccgggtatagcctgaccaccatctcccacgtactgattgggatcgccagcgtggccttccccgtgggcgatcctctgcgtccgtacgtcatcctgaccctggtcgtcgtgttcgtaggcagcatgcaaaccttcctgaacgtggccacgtgggtgatgctgagcgagctctttccgctggcgatgcgggggttcgccattggcatcagcgtcttctttctctggatcgcgaacgcgttcctgggcctgttttttcctacgatcatggaagccgtgggcctgacgggcacctttttcatgttcgccggtatcggggtcgtagccctgattttcatttacacccaagtgccggaaacccgggggcgcaccctggaagaaatcgacgaggacgtgaccagcggcgtcatcttcaacaaggacattcgcaagggcaaggtccattgactcgtctacaccatcaataagaaaaagaggacgcaaagggatggaacagcgcaaacgcatcaagacactgggatcgttggccttgcttgcacttgtaggcagcagcggtacacaggctgccgaggctttttccagcgaatccaaatggatgaccggcgactggggcggcacccggaccgagctgctggacaagggctatgacttcaccctcgattatgtgggtgaggtggctggcaacctgcatggcggctacaacgacgacaagacggcacgctacagcgaccagttcgccctcggcgcgcatctggacttgcagaagatactgggctggcatgatgccgagttcaagctggcaatcaccgagcgaagcggtcgcaacctgtccaacgaccgcatcagcgacccgcgcgccgggcagttcagctcggtgcaggaggtgtggggccgtggccagacctggcgcctgacccagatgtggatcaagcagaagtacttcgacggcgcgctggacgtgaaatttggccgttttggcgagggcgaggacttcaacagcttcccttgcgacttccagaacctggccttctgcggctcgcaggtgggcaactgggtgggcggcatctggtacaactggccggtcagccagtgggcgctgcgggtgaagtacaacatcacgcctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctttccttcttcactgtcccttctagaacgttctagaagggacagtgaagaaggaacacccgctcgcgggtgggcctactctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggtcaggcgtccttttgcttggtgccgaagatcttgtcaccggcatcacccaggcctggcacgatgtagccgtgctcgttcaggcgctggtcgatcgaggcggtgtagatcttcacgtccgggtgggctttttccaccacctcgatgccttctggtgcggcgaccagcaccatggcgcgaatctctttgcagccggcctttttcagcaggtcgatggtggcaaccatcgagccgccggtggccagcatcgggtcgatgatcagggccaggcgctggttgatgtccggcgcgagcttttccagataggtgtgggcttcgagggtttcttcgtttcgggcaacgccgacggcgctgaccttggcccccgggatcaggctgagcacgccgtcgagcatgccgatgccggcgcgcaggatcggtactacggtgatcttcttgccggcgattttttcaaccgagaccttgccacaccagccgtcgatctcgtaggtttcgaggggcaggtcctgggtggcttcatacgtcaggagcgcgccgacttcctgggcgagttcgcgaaaattcttggtgctgatatcggcacggcgcatcaggccaagcttgtggcggatcagcggatggcggatctcacgagtgggcataggggagggctccgaaaggcgggcaaaaaaaccgcgctagattaatctattcagcctgtgctgtcgtctggtcattctggacgttagtccataaatgcttgatctgtgacgagcggatgcgtacctttgcccgcttttccaaaatgctagccggctacgtatcgataagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctg

SEQ ID NO: 53 - DNA, 4678 bp, Artificial Sequence
  knockout construct
gcatcgcttcgttacctgacgaggcgggcacgctgtttagacttcatgctacgcaagcgatcgacttgcacggcataacaacaagaaaggtgctccgatgaattccacgcttcgtctcgctgccgcaatttcctttgcctcgttaatccccttgggtgcccaggctgctgacgccaaaggcagtgtcgaagtggtgcactggtggacctccggtggtgaaaaagcggcggtcgatgtgctcaaggcccaggtcgaaaaagacggcttcatctggaaggacggcgccgtcgccggcggcggtggtgcaacggccatgaccgtgctcaaaagccgcgcagtggccggcaacccgccgggcgtcgcgcagatcaaaggcccggacatccaggactgggcggccaccggcctgctcgacgccgatgtgctcaaggatgtggccaaggaaggaaagtgggactcgctgctcgacaagaaagtggccgacaccgtgaagtacgacggtgactacgttgccgtaccggtgaatatccaccgcatcaactggctgtggatcaaccccgaggtgttcaaaaaagccggcatcgacaaggcgcccaccaccctcgacgaattctacgccgccgccgacaagctcaaggctgccggtttcatcccgctcgcccatggtgggcaaccctggcaggacagcaccgtgttcgaaagcgtggtgctgtcggtgatgggcgtcgatggctacaagaaggccttggtcgacctcgacagcgcaacgctgaccgggccgcagatggtcaaggcgctgaccgagttgaagaaagtcgccacctacatggacccggacggcaagggccaggactggaacctggaagccgccaaggtcattaacggcaaggccggcatgcagatcatgggcgactgggccaagagcgaatggaccctggcgaagaaaactgctggcaaggattaccagtgcgtgccgttccccggtactgacaagtccttcctgtacaacatcgactcgttggtggtgttcaagcagaacaacgctggcacctctgctggtcagcaggacatcgcccgcaaggtgttgggtgaggacttccagaaggtcttcagcatcaacaagggttcgatcccggtacgcaacgacatgcttgccgacatgggcaagtatggtttcgatgcctgtgctcaaacctccgccaaggacttcctggctgacgccaaaacgggcggcctgcaaccgagcatggcgcacaacatggccaccacgctggccgtgcagggcgcgttcttcgatgtggtgaccaactacatcaacgaccccaaggccgacccggccgatgcggcgaagaagctggcggcggctatcaaggctgcccagtaacggctgttcgcgggcacgcccgctcccagatgggccctgctcaccactgttcctggtcatgacgccggacctgtgggagcgggcttgcccgcgaagcggtgcgtagcaccgccactacctcgggatccaatacatggcaagcacctttatccaggccgacagcccggagaaaagcaaaaaactgcccccgctgaccgaaggcccgtaccgcaagcgcttgttctatgtggccctggtagctaccttcggcggtctgctcttcggctacgacaccggcgtcatcaatggtgcgctgaaccccatgacgcgcgagctggggctgaccgcattcaccgagggcgtggtgaccagcagcctgctgtttggggccgcggcgggcgccatgttcttcggtcgcatcagcgacaactgggggcgccgcaaaaccatcatctcgctggctgtcgccttcttcgtggggaccatgatctgcgtattcgccccatcgttcgccgtgatggtggtgggccgcgtgttgctgggcctggcggtcggcggcgcgagcacggtggtgcccgtctacttggccgaactggcgccgttcgagatccggggctcgctggccggccgcaacgaactgatgatcgtggtggggcaactggccgcattcgtgattaatgccatcatcggcaatgtgttcggccatcacgacggggtctggcgctacatgctggccatcgcagccatcccggccattgccctgttcttcggcatgctgcgtgtacctgagtccccccgctggctggtggagcgcgggcgtattgacgaagcccgcgcggtcctggagacgatccgtccattggaacgcgcccacgcggaggtggccgacgtcgaacacctggcccgcgaggagcacgctgtgtcggagaagtcgatgggcctgcgcgaaatcctgagctcgaagtggctggtgcgcatcctgctcgtgggcattggtctcggtgtggcgcagcaactcaccggtatcaacagcatcatgtactacggtcaggtcgtcttgatcgaagccggctttagcgagaacgctgcgctgatcgccaacgtggcccccggcgtgattgccgtggtcggcgcctttatcgcgttgtggatgatggatcgtattaaccgtcgtaccaccttgatcaccgggtatagcctgaccaccatctcccacgtactgattgggatcgccagcgtggccttccccgtgggcgatcctctgcgtccgtacgtcatcctgaccctggtcgtcgtgttcgtaggcagcatgcaaaccttcctgaacgtggccacgtgggtgatgctgagcgagctctttccgctggcgatgcgggggttcgccattggcatcagcgtcttctttctctggatcgcgaacgcgttcctgggcctgttttttcctacgatcatggaagccgtgggcctgacgggcacctttttcatgttcgccggtatcggggtcgtagccctgattttcatttacacccaagtgccggaaacccgggggcgcaccctggaagaaatcgacgaggacgtgaccagcggcgtcatcttcaacaaggacattcgcaagggcaaggtccattgactcgtctacaccatcaataagaaaaagaggacgcaaagggatggaacagcgcaaacgcatcaagacactgggatcgttggccttgcttgcacttgtaggcagcagcggtacacaggctgccgaggctttttccagcgaatccaaatggatgaccggcgactggggcggcacccggaccgagctgctggacaagggctatgacttcaccctcgattatgtgggtgaggtggctggcaacctgcatggcggctacaacgacgacaagacggcacgctacagcgaccagttcgccctcggcgcgcatctggacttgcagaagatactgggctggcatgatgccgagttcaagctggcaatcaccgagcgaagcggtcgcaacctgtccaacgaccgcatcagcgacccgcgcgccgggcagttcagctcggtgcaggaggtgtggggccgtggccagacctggcgcctgacccagatgtggatcaagcagaagtacttcgacggcgcgctggacgtgaaatttggccgttttggcgagggcgaggacttcaacagcttcccttgcgacttccagaacctggccttctgcggctcgcaggtgggcaactgggtgggcggcatctggtacaactggccggtcagccagtgggcgctgcgggtgaagtacaacatcacgccggagttcttcgtacaggtcggggccttcgagcagaacccttccaacctggaaaccggcaacggcttcaagctcagcggcagtggtaccaagggggcgatcttgccggtggaagcggtgtggtcgcccaaggtcaatggcctgccgggcgagtaccgcctgggttactactacagcacggccaaggctgacgatgtgtacgacgacgtcaacggcaacccgcaggcgctgacaggtgaagccttcaagtcgcactccagcaagcacggatggtgggtggtggcgcagcagcaggtcactgcccatggcggcgacgtcaaccggggcctcagcctgttcgccaacttcaccgtgcacgacaaggccaccaacgtggtcgacaactaccagcaggtggggctggtctacaaaggcgctttcgacgcccggcccaaggatgacatcggcttcggcgtggcgcgtattcatgtgaatgacgacgtgaagaagcgcgccgaactgctcaacgcacagagcggcatcaacgattacgacaaccctggtttcgtgccgctgcagcgtaccgaatacaacgcagagctctactacggcttccacgttaccaactggctgaccgtgaggcccaacctgcagtacatcaagagccctggcggggtggacgaggtggataacgcgctggtcgctggcttgaagattcagtcgtcattctgaggcaacttgttgtaaatttacgccaatccattttcggttcccggcacccactggtctgcagtgggtgttggggataggccgagacagcgctatctcaaacaacaagagctctgaaccatgcccgaacatccgctccatcgcttcttctcctcgcagcggccccggccgacattcgagtgggagcgttatcagcagcgcgatgtcctgatcatcgaccacccccgttgccaggcggtgttcagccgccagggcgcgcagttgctgcactttcaaccagcgggtgaacggccgtggctgtggt

SEQ ID NO: 54 - DNA, 44 bp, Artificial Sequence
  primer
actacctcgggatccaatacatgaatgtgatcggtatcactctc

SEQ ID NO: 55 - DNA, 20 bp, Artificial Sequence
  primer
tcagtgccccagcgcttcgg

SEQ ID NO: 56 - DNA, 47 bp, Artificial Sequence
  primer
ccgaagcgctggggcactgactcgtctacaccatcaataagaaaaag

SEQ ID NO: 57 - DNA, 1517 bp, Artificial Sequence
  PCR product
actacctcgggatccaatacatgaatgtgatcggtatcactctcctgccgagagggcgcatcatgtcacacggtccggtaagcgacgacaccccgtcgatcttcggtgacgacgatcaggcggcctcctccggtcgcaccgcggtccggatcgcggcggtcgcggccctcggcggtctcctgttcggctacgacagcgccgtcatcaacggtgccgtggactcgatccaggaggacttcggcatcggcaattacgccctgggccttgccgtggcgtcggcgctgctgggtgctgccgcaggcgctctgtcggccggccgtatcgccgaccgcatcgggcgcatcgcggtgatgaaaatcgccgccgtcttgttcttcatcagcgccttcggaacgggtttcgcacccgaaacggtcactctcgtggtgttccgcatcgtcggtggtatcggcgtgggtgtggcatcggtgatcgcacccgcctacattgccgagacctctccgccgggaatccggggacgcctcggatcgctgcagcaactggccatcgtgctgggcatcttcacgtcctttgtcgtcaactggctgttgcagtgggcggcgggcggtcccaacgaggtgctggcgatgggactcgacgcatggcgctggatgttcctggccatggccgtaccggccgtcctgtatggggcgctcgcgttcaccatcccggagtcgccgcgttatctcgttgccacacacaagatcccagaagcacgccgggtgctgagcatgctgctcgggcagaagaacctggagatcaccatcacgcgtatccgcgacacccttgagcgcgaggacaaaccgtcgtggcgtgatctgaagaaacccaccggcgggatctacgggatcgtgtgggtcggtctcggtctgtcgatcttccagcagttcgtcggtatcaatgtgatcttctactactcgaatgtgctgtggcaggccgtcggtttcagcgccgaccagtccgcgatctacaccgtgatcacgtcggtggtcaacgtgctgacgacgttgatcgcgatcgcgctgatcgacaagatcggccgcaaaccgctcctgctgatcggctcgtccggcatggcggtcacgctggccaccatggcggtcatcttcgccaatgccacggtcaagcccgacggcacgcccgacctgcccggcgcgtccggcttgatcgcactcatcgcggcgaacctgttcgtggtcgctttcggtatgtcgtgggggccggtggtctgggtgctgctgggggagatgttccccaaccgcttccgtgcggccgcgctgggcctggcggcggccgggcagtgggccgcgaactggttgatcaccgtcagcttccccgagctgcgcaaccacctgggcctggcctacggcttctatgccctctgtgcggtgctgtcgttcctcttcgtgagcaagtgggtcgaggagaccaggggtaagaatctggaggacatgcacgccgaagcgctggggcactga

SEQ ID NO: 58 - DNA, 710 bp, Artificial Sequence
  PCR product
ccgaagcgctggggcactgactcgtctacaccatcaataagaaaaagaggacgcaaagggatggaacagcgcaaacgcatcaagacactgggatcgttggccttgcttgcacttgtaggcagcagcggtacacaggctgccgaggctttttccagcgaatccaaatggatgaccggcgactggggcggcacccggaccgagctgctggacaagggctatgacttcaccctcgattatgtgggtgaggtggctggcaacctgcatggcggctacaacgacgacaagacggcacgctacagcgaccagttcgccctcggcgcgcatctggacttgcagaagatactgggctggcatgatgccgagttcaagctggcaatcaccgagcgaagcggtcgcaacctgtccaacgaccgcatcagcgacccgcgcgccgggcagttcagctcggtgcaggaggtgtggggccgtggccagacctggcgcctgacccagatgtggatcaagcagaagtacttcgacggcgcgctggacgtgaaatttggccgttttggcgagggcgaggacttcaacagcttcccttgcgacttccagaacctggccttctgcggctcgcaggtgggcaactgggtgggcggcatctggtacaactggccggtcagccagtgggcgctgcgggtgaagtacaacatcacgcctgcaggcatgcaagc

SEQ ID NO: 59 - DNA, 7637 bp, Artificial Sequence
  vector
atggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgggctcgatcccctcgcgagttggttcagctgctgcctgaggctggacgacctcgcggagttctaccggcagtgcaaatccgtcggcatccaggaaaccagcagcggctatccgcgcatccatgcccccgaactgcaggagtggggaggcacgatggccgctttggtcccggccgccaaggtcattaacggcaaggccggcatgcagatcatgggcgactgggccaagagcgaatggaccctggcgaagaaaactgctggcaaggattaccagtgcgtgccgttccccggtactgacaagtccttcctgtacaacatcgactcgttggtggtgttcaagcagaacaacgctggcacctctgctggtcagcaggacatcgcccgcaaggtgttgggtgaggacttccagaaggtcttcagcatcaacaagggttcgatcccggtacgcaacgacatgcttgccgacatgggcaagtatggtttcgatgcctgtgctcaaacctccgccaaggacttcctggctgacgccaaaacgggcggcctgcaaccgagcatggcgcacaacatggccaccacgctggccgtgcagggcgcgttcttcgatgtggtgaccaactacatcaacgaccccaaggccgacccggccgatgcggcgaagaagctggcggcggctatcaaggctgcccagtaacggctgttcgcgggcacgcccgctcccagatgggccctgctcaccactgttcctggtcatgacgccggacctgtgggagcgggcttgcccgcgaagcggtgcgtagcaccgccactacctcgggatccaatacatgaatgtgatcggtatcactctcctgccgagagggcgcatcatgtcacacggtccggtaagcgacgacaccccgtcgatcttcggtgacgacgatcaggcggcctcctccggtcgcaccgcggtccggatcgcggcggtcgcggccctcggcggtctcctgttcggctacgacagcgccgtcatcaacggtgccgtggactcgatccaggaggacttcggcatcggcaattacgccctgggccttgccgtggcgtcggcgctgctgggtgctgccgcaggcgctctgtcggccggccgtatcgccgaccgcatcgggcgcatcgcggtgatgaaaatcgccgccgtcttgttcttcatcagcgccttcggaacgggtttcgcacccgaaacggtcactctcgtggtgttccgcatcgtcggtggtatcggcgtgggtgtggcatcggtgatcgcacccgcctacattgccgagacctctccgccgggaatccggggacgcctcggatcgctgcagcaactggccatcgtgctgggcatcttcacgtcctttgtcgtcaactggctgttgcagtgggcggcgggcggtcccaacgaggtgctggcgatgggactcgacgcatggcgctggatgttcctggccatggccgtaccggccgtcctgtatggggcgctcgcgttcaccatcccggagtcgccgcgttatctcgttgccacacacaagatcccagaagcacgccgggtgctgagcatgctgctcgggcagaagaacctggagatcaccatcacgcgtatccgcgacacccttgagcgcgaggacaaaccgtcgtggcgtgatctgaagaaacccaccggcgggatctacgggatcgtgtgggtcggtctcggtctgtcgatcttccagcagttcgtcggtatcaatgtgatcttctactactcgaatgtgctgtggcaggccgtcggtttcagcgccgaccagtccgcgatctacaccgtgatcacgtcggtggtcaacgtgctgacgacgttgatcgcgatcgcgctgatcgacaagatcggccgcaaaccgctcctgctgatcggctcgtccggcatggcggtcacgctggccaccatggcggtcatcttcgccaatgccacggtcaagcccgacggcacgcccgacctgcccggcgcgtccggcttgatcgcactcatcgcggcgaacctgttcgtggtcgctttcggtatgtcgtgggggccggtggtctgggtgctgctgggggagatgttccccaaccgcttccgtgcggccgcgctgggcctggcggcggccgggcagtgggccgcgaactggttgatcaccgtcagcttccccgagctgcgcaaccacctgggcctggcctacggcttctatgccctctgtgcggtgctgtcgttcctcttcgtgagcaagtgggtcgaggagaccaggggtaagaatctggaggacatgcacgccgaagcgctggggcactgactcgtctacaccatcaataagaaaaagaggacgcaaagggatggaacagcgcaaacgcatcaagacactgggatcgttggccttgcttgcacttgtaggcagcagcggtacacaggctgccgaggctttttccagcgaatccaaatggatgaccggcgactggggcggcacccggaccgagctgctggacaagggctatgacttcaccctcgattatgtgggtgaggtggctggcaacctgcatggcggctacaacgacgacaagacggcacgctacagcgaccagttcgccctcggcgcgcatctggacttgcagaagatactgggctggcatgatgccgagttcaagctggcaatcaccgagcgaagcggtcgcaacctgtccaacgaccgcatcagcgacccgcgcgccgggcagttcagctcggtgcaggaggtgtggggccgtggccagacctggcgcctgacccagatgtggatcaagcagaagtacttcgacggcgcgctggacgtgaaatttggccgttttggcgagggcgaggacttcaacagcttcccttgcgacttccagaacctggccttctgcggctcgcaggtgggcaactgggtgggcggcatctggtacaactggccggtcagccagtgggcgctgcgggtgaagtacaacatcacgcctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctttccttcttcactgtcccttctagaacgttctagaagggacagtgaagaaggaacacccgctcgcgggtgggcctactctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggtcaggcgtccttttgcttggtgccgaagatcttgtcaccggcatcacccaggcctggcacgatgtagccgtgctcgttcaggcgctggtcgatcgaggcggtgtagatcttcacgtccgggtgggctttttccaccacctcgatgccttctggtgcggcgaccagcaccatggcgcgaatctctttgcagccggcctttttcagcaggtcgatggtggcaaccatcgagccgccggtggccagcatcgggtcgatgatcagggccaggcgctggttgatgtccggcgcgagcttttccagataggtgtgggcttcgagggtttcttcgtttcgggcaacgccgacggcgctgaccttggcccccgggatcaggctgagcacgccgtcgagcatgccgatgccggcgcgcaggatcggtactacggtgatcttcttgccggcgattttttcaaccgagaccttgccacaccagccgtcgatctcgtaggtttcgaggggcaggtcctgggtggcttcatacgtcaggagcgcgccgacttcctgggcgagttcgcgaaaattcttggtgctgatatcggcacggcgcatcaggccaagcttgtggcggatcagcggatggcggatctcacgagtgggcataggggagggctccgaaaggcgggcaaaaaaaccgcgctagattaatctattcagcctgtgctgtcgtctggtcattctggacgttagtccataaatgcttgatctgtgacgagcggatgcgtacctttgcccgcttttccaaaatgctagccggctacgtatcgataagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctg

SEQ ID NO: 60 - DNA, 4699 bp, Artificial Sequence
  knockout construct
gcatcgcttcgttacctgacgaggcgggcacgctgtttagacttcatgctacgcaagcgatcgacttgcacggcataacaacaagaaaggtgctccgatgaattccacgcttcgtctcgctgccgcaatttcctttgcctcgttaatccccttgggtgcccaggctgctgacgccaaaggcagtgtcgaagtggtgcactggtggacctccggtggtgaaaaagcggcggtcgatgtgctcaaggcccaggtcgaaaaagacggcttcatctggaaggacggcgccgtcgccggcggcggtggtgcaacggccatgaccgtgctcaaaagccgcgcagtggccggcaacccgccgggcgtcgcgcagatcaaaggcccggacatccaggactgggcggccaccggcctgctcgacgccgatgtgctcaaggatgtggccaaggaaggaaagtgggactcgctgctcgacaagaaagtggccgacaccgtgaagtacgacggtgactacgttgccgtaccggtgaatatccaccgcatcaactggctgtggatcaaccccgaggtgttcaaaaaagccggcatcgacaaggcgcccaccaccctcgacgaattctacgccgccgccgacaagctcaaggctgccggtttcatcccgctcgcccatggtgggcaaccctggcaggacagcaccgtgttcgaaagcgtggtgctgtcggtgatgggcgtcgatggctacaagaaggccttggtcgacctcgacagcgcaacgctgaccgggccgcagatggtcaaggcgctgaccgagttgaagaaagtcgccacctacatggacccggacggcaagggccaggactggaacctggaagccgccaaggtcattaacggcaaggccggcatgcagatcatgggcgactgggccaagagcgaatggaccctggcgaagaaaactgctggcaaggattaccagtgcgtgccgttccccggtactgacaagtccttcctgtacaacatcgactcgttggtggtgttcaagcagaacaacgctggcacctctgctggtcagcaggacatcgcccgcaaggtgttgggtgaggacttccagaaggtcttcagcatcaacaagggttcgatcccggtacgcaacgacatgcttgccgacatgggcaagtatggtttcgatgcctgtgctcaaacctccgccaaggacttcctggctgacgccaaaacgggcggcctgcaaccgagcatggcgcacaacatggccaccacgctggccgtgcagggcgcgttcttcgatgtggtgaccaactacatcaacgaccccaaggccgacccggccgatgcggcgaagaagctggcggcggctatcaaggctgcccagtaacggctgttcgcgggcacgcccgctcccagatgggccctgctcaccactgttcctggtcatgacgccggacctgtgggagcgggcttgcccgcgaagcggtgcgtagcaccgccactacctcgggatccaatacatgaatgtgatcggtatcactctcctgccgagagggcgcatcatgtcacacggtccggtaagcgacgacaccccgtcgatcttcggtgacgacgatcaggcggcctcctccggtcgcaccgcggtccggatcgcggcggtcgcggccctcggcggtctcctgttcggctacgacagcgccgtcatcaacggtgccgtggactcgatccaggaggacttcggcatcggcaattacgccctgggccttgccgtggcgtcggcgctgctgggtgctgccgcaggcgctctgtcggccggccgtatcgccgaccgcatcgggcgcatcgcggtgatgaaaatcgccgccgtcttgttcttcatcagcgccttcggaacgggtttcgcacccgaaacggtcactctcgtggtgttccgcatcgtcggtggtatcggcgtgggtgtggcatcggtgatcgcacccgcctacattgccgagacctctccgccgggaatccggggacgcctcggatcgctgcagcaactggccatcgtgctgggcatcttcacgtcctttgtcgtcaactggctgttgcagtgggcggcgggcggtcccaacgaggtgctggcgatgggactcgacgcatggcgctggatgttcctggccatggccgtaccggccgtcctgtatggggcgctcgcgttcaccatcccggagtcgccgcgttatctcgttgccacacacaagatcccagaagcacgccgggtgctgagcatgctgctcgggcagaagaacctggagatcaccatcacgcgtatccgcgacacccttgagcgcgaggacaaaccgtcgtggcgtgatctgaagaaacccaccggcgggatctacgggatcgtgtgggtcggtctcggtctgtcgatcttccagcagttcgtcggtatcaatgtgatcttctactactcgaatgtgctgtggcaggccgtcggtttcagcgccgaccagtccgcgatctacaccgtgatcacgtcggtggtcaacgtgctgacgacgttgatcgcgatcgcgctgatcgacaagatcggccgcaaaccgctcctgctgatcggctcgtccggcatggcggtcacgctggccaccatggcggtcatcttcgccaatgccacggtcaagcccgacggcacgcccgacctgcccggcgcgtccggcttgatcgcactcatcgcggcgaacctgttcgtggtcgctttcggtatgtcgtgggggccggtggtctgggtgctgctgggggagatgttccccaaccgcttccgtgcggccgcgctgggcctggcggcggccgggcagtgggccgcgaactggttgatcaccgtcagcttccccgagctgcgcaaccacctgggcctggcctacggcttctatgccctctgtgcggtgctgtcgttcctcttcgtgagcaagtgggtcgaggagaccaggggtaagaatctggaggacatgcacgccgaagcgctggggcactgactcgtctacaccatcaataagaaaaagaggacgcaaagggatggaacagcgcaaacgcatcaagacactgggatcgttggccttgcttgcacttgtaggcagcagcggtacacaggctgccgaggctttttccagcgaatccaaatggatgaccggcgactggggcggcacccggaccgagctgctggacaagggctatgacttcaccctcgattatgtgggtgaggtggctggcaacctgcatggcggctacaacgacgacaagacggcacgctacagcgaccagttcgccctcggcgcgcatctggacttgcagaagatactgggctggcatgatgccgagttcaagctggcaatcaccgagcgaagcggtcgcaacctgtccaacgaccgcatcagcgacccgcgcgccgggcagttcagctcggtgcaggaggtgtggggccgtggccagacctggcgcctgacccagatgtggatcaagcagaagtacttcgacggcgcgctggacgtgaaatttggccgttttggcgagggcgaggacttcaacagcttcccttgcgacttccagaacctggccttctgcggctcgcaggtgggcaactgggtgggcggcatctggtacaactggccggtcagccagtgggcgctgcgggtgaagtacaacatcacgccggagttcttcgtacaggtcggggccttcgagcagaacccttccaacctggaaaccggcaacggcttcaagctcagcggcagtggtaccaagggggcgatcttgccggtggaagcggtgtggtcgcccaaggtcaatggcctgccgggcgagtaccgcctgggttactactacagcacggccaaggctgacgatgtgtacgacgacgtcaacggcaacccgcaggcgctgacaggtgaagccttcaagtcgcactccagcaagcacggatggtgggtggtggcgcagcagcaggtcactgcccatggcggcgacgtcaaccggggcctcagcctgttcgccaacttcaccgtgcacgacaaggccaccaacgtggtcgacaactaccagcaggtggggctggtctacaaaggcgctttcgacgcccggcccaaggatgacatcggcttcggcgtggcgcgtattcatgtgaatgacgacgtgaagaagcgcgccgaactgctcaacgcacagagcggcatcaacgattacgacaaccctggtttcgtgccgctgcagcgtaccgaatacaacgcagagctctactacggcttccacgttaccaactggctgaccgtgaggcccaacctgcagtacatcaagagccctggcggggtggacgaggtggataacgcgctggtcgctggcttgaagattcagtcgtcattctgaggcaacttgttgtaaatttacgccaatccattttcggttcccggcacccactggtctgcagtgggtgttggggataggccgagacagcgctatctcaaacaacaagagctctgaaccatgcccgaacatccgctccatcgcttcttctcctcgcagcggccccggccgacattcgagtgggagcgttatcagcagcgcgatgtcctgatcatcgaccacccccgttgccaggcggtgttcagccgccagggcgcgcagttgctgcactttcaaccagcgggtgaacggccgtggctgtggt

SEQ ID NO: 61 - DNA, 39 bp, Artificial Sequence
  primer
tacctcgggatccaatacatggacttgttgctggctctg

SEQ ID NO: 62 - DNA, 22 bp, Artificial Sequence
  primer
ctagctgttggtcttggcgatg

SEQ ID NO: 63 - DNA, 47 bp, Artificial Sequence
  primer
tcgccaagaccaacagctagctcgtctacaccatcaataagaaaaag

SEQ ID NO: 64 - DNA, 882 bp, Artificial Sequence
  PCR product
tacctcgggatccaatacatggacttgttgctggctctgctgccggctttgttctggggttcgatcgtgctgttcaacgtcaaactgggcggcggcccctacagccagaccctgggcaccaccatcggggctctgatcgtctcgattgtcatttatttctttgtgcagccagtgctgagcctgcgcatcttcatcgtcggcatcgtgagcggcctgttctggagcctgggtcaggccaaccagctgaagtcgatccagttgatgggggtaagcaagaccatgccaatttccaccggcatgcagttggtgtcgacctcgctgtttggcgtgatcgtgtttcgggaatggtcgaccccgatcgccatcacgctcggggtcctcgccctgatcttcatcatcgtgggcatcatcctcacctcgctggaggataaaaacgacaaaaaagaaggggagccgtccaacctgaaaaagggtatcctgatcctgctggtgtcgaccctcggctacctggtgtatgtagtggtggcccgcctgttcaacgtctccggttggtcggcgttgctgccgcaggcaatcggcatggtggtgggcggcctggtcttgacctaccggcacaaaccattcaacaagtatgcgattcgtaatatcttgccgggcctgatctgggctggcggcaacatgttcctgtttattagccagccgcgcgtgggtgtcgccacctcctttagcctgagccagatgggcatcgtaatctcgaccttgggcggcatctttatcctccgtgagaagaagacgaagcggcagctgatcgcgatcgccatcggcatcatcttgatcatcgcggccgccgtgttcctgggcatcgccaagaccaacagctag

SEQ ID NO: 65 - DNA, 710 bp, Artificial Sequence
  PCR product
tcgccaagaccaacagctagctcgtctacaccatcaataagaaaaagaggacgcaaagggatggaacagcgcaaacgcatcaagacactgggatcgttggccttgcttgcacttgtaggcagcagcggtacacaggctgccgaggctttttccagcgaatccaaatggatgaccggcgactggggcggcacccggaccgagctgctggacaagggctatgacttcaccctcgattatgtgggtgaggtggctggcaacctgcatggcggctacaacgacgacaagacggcacgctacagcgaccagttcgccctcggcgcgcatctggacttgcagaagatactgggctggcatgatgccgagttcaagctggcaatcaccgagcgaagcggtcgcaacctgtccaacgaccgcatcagcgacccgcgcgccgggcagttcagctcggtgcaggaggtgtggggccgtggccagacctggcgcctgacccagatgtggatcaagcagaagtacttcgacggcgcgctggacgtgaaatttggccgttttggcgagggcgaggacttcaacagcttcccttgcgacttccagaacctggccttctgcggctcgcaggtgggcaactgggtgggcggcatctggtacaactggccggtcagccagtgggcgctgcgggtgaagtacaacatcacgcctgcaggcatgcaagc

SEQ ID NO: 66 - DNA, 7004 bp, Artificial Sequence
  vector
atggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgggctcgatcccctcgcgagttggttcagctgctgcctgaggctggacgacctcgcggagttctaccggcagtgcaaatccgtcggcatccaggaaaccagcagcggctatccgcgcatccatgcccccgaactgcaggagtggggaggcacgatggccgctttggtcccggccgccaaggtcattaacggcaaggccggcatgcagatcatgggcgactgggccaagagcgaatggaccctggcgaagaaaactgctggcaaggattaccagtgcgtgccgttccccggtactgacaagtccttcctgtacaacatcgactcgttggtggtgttcaagcagaacaacgctggcacctctgctggtcagcaggacatcgcccgcaaggtgttgggtgaggacttccagaaggtcttcagcatcaacaagggttcgatcccggtacgcaacgacatgcttgccgacatgggcaagtatggtttcgatgcctgtgctcaaacctccgccaaggacttcctggctgacgccaaaacgggcggcctgcaaccgagcatggcgcacaacatggccaccacgctggccgtgcagggcgcgttcttcgatgtggtgaccaactacatcaacgaccccaaggccgacccggccgatgcggcgaagaagctggcggcggctatcaaggctgcccagtaacggctgttcgcgggcacgcccgctcccagatgggccctgctcaccactgttcctggtcatgacgccggacctgtgggagcgggcttgcccgcgaagcggtgcgtagcaccgccactacctcgggatccaatacatggacttgttgctggctctgctgccggctttgttctggggttcgatcgtgctgttcaacgtcaaactgggcggcggcccctacagccagaccctgggcaccaccatcggggctctgatcgtctcgattgtcatttatttctttgtgcagccagtgctgagcctgcgcatcttcatcgtcggcatcgtgagcggcctgttctggagcctgggtcaggccaaccagctgaagtcgatccagttgatgggggtaagcaagaccatgccaatttccaccggcatgcagttggtgtcgacctcgctgtttggcgtgatcgtgtttcgggaatggtcgaccccgatcgccatcacgctcggggtcctcgccctgatcttcatcatcgtgggcatcatcctcacctcgctggaggataaaaacgacaaaaaagaaggggagccgtccaacctgaaaaagggtatcctgatcctgctggtgtcgaccctcggctacctggtgtatgtagtggtggcccgcctgttcaacgtctccggttggtcggcgttgctgccgcaggcaatcggcatggtggtgggcggcctggtcttgacctaccggcacaaaccattcaacaagtatgcgattcgtaatatcttgccgggcctgatctgggctggcggcaacatgttcctgtttattagccagccgcgcgtgggtgtcgccacctcctttagcctgagccagatgggcatcgtaatctcgaccttgggcggcatctttatcctccgtgagaagaagacgaagcggcagctgatcgcgatcgccatcggcatcatcttgatcatcgcggccgccgtgttcctgggcatcgccaagaccaacagctagctcgtctacaccatcaataagaaaaagaggacgcaaagggatggaacagcgcaaacgcatcaagacactgggatcgttggccttgcttgcacttgtaggcagcagcggtacacaggctgccgaggctttttccagcgaatccaaatggatgaccggcgactggggcggcacccggaccgagctgctggacaagggctatgacttcaccctcgattatgtgggtgaggtggctggcaacctgcatggcggctacaacgacgacaagacggcacgctacagcgaccagttcgccctcggcgcgcatctggacttgcagaagatactgggctggcatgatgccgagttcaagctggcaatcaccgagcgaagcggtcgcaacctgtccaacgaccgcatcagcgacccgcgcgccgggcagttcagctcggtgcaggaggtgtggggccgtggccagacctggcgcctgacccagatgtggatcaagcagaagtacttcgacggcgcgctggacgtgaaatttggccgttttggcgagggcgaggacttcaacagcttcccttgcgacttccagaacctggccttctgcggctcgcaggtgggcaactgggtgggcggcatctggtacaactggccggtcagccagtgggcgctgcgggtgaagtacaacatcacgcctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctttccttcttcactgtcccttctagaacgttctagaagggacagtgaagaaggaacacccgctcgcgggtgggcctactctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggtcaggcgtccttttgcttggtgccgaagatcttgtcaccggcatcacccaggcctggcacgatgtagccgtgctcgttcaggcgctggtcgatcgaggcggtgtagatcttcacgtccgggtgggctttttccaccacctcgatgccttctggtgcggcgaccagcaccatggcgcgaatctctttgcagccggcctttttcagcaggtcgatggtggcaaccatcgagccgccggtggccagcatcgggtcgatgatcagggccaggcgctggttgatgtccggcgcgagcttttccagataggtgtgggcttcgagggtttcttcgtttcgggcaacgccgacggcgctgaccttggcccccgggatcaggctgagcacgccgtcgagcatgccgatgccggcgcgcaggatcggtactacggtgatcttcttgccggcgattttttcaaccgagaccttgccacaccagccgtcgatctcgtaggtttcgaggggcaggtcctgggtggcttcatacgtcaggagcgcgccgacttcctgggcgagttcgcgaaaattcttggtgctgatatcggcacggcgcatcaggccaagcttgtggcggatcagcggatggcggatctcacgagtgggcataggggagggctccgaaaggcgggcaaaaaaaccgcgctagattaatctattcagcctgtgctgtcgtctggtcattctggacgttagtccataaatgcttgatctgtgacgagcggatgcgtacctttgcccgcttttccaaaatgctagccggctacgtatcgataagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctg

SEQ ID NO: 67 - DNA, 4066 bp, Artificial Sequence
  knockout construct
gcatcgcttcgttacctgacgaggcgggcacgctgtttagacttcatgctacgcaagcgatcgacttgcacggcataacaacaagaaaggtgctccgatgaattccacgcttcgtctcgctgccgcaatttcctttgcctcgttaatccccttgggtgcccaggctgctgacgccaaaggcagtgtcgaagtggtgcactggtggacctccggtggtgaaaaagcggcggtcgatgtgctcaaggcccaggtcgaaaaagacggcttcatctggaaggacggcgccgtcgccggcggcggtggtgcaacggccatgaccgtgctcaaaagccgcgcagtggccggcaacccgccgggcgtcgcgcagatcaaaggcccggacatccaggactgggcggccaccggcctgctcgacgccgatgtgctcaaggatgtggccaaggaaggaaagtgggactcgctgctcgacaagaaagtggccgacaccgtgaagtacgacggtgactacgttgccgtaccggtgaatatccaccgcatcaactggctgtggatcaaccccgaggtgttcaaaaaagccggcatcgacaaggcgcccaccaccctcgacgaattctacgccgccgccgacaagctcaaggctgccggtttcatcccgctcgcccatggtgggcaaccctggcaggacagcaccgtgttcgaaagcgtggtgctgtcggtgatgggcgtcgatggctacaagaaggccttggtcgacctcgacagcgcaacgctgaccgggccgcagatggtcaaggcgctgaccgagttgaagaaagtcgccacctacatggacccggacggcaagggccaggactggaacctggaagccgccaaggtcattaacggcaaggccggcatgcagatcatgggcgactgggccaagagcgaatggaccctggcgaagaaaactgctggcaaggattaccagtgcgtgccgttccccggtactgacaagtccttcctgtacaacatcgactcgttggtggtgttcaagcagaacaacgctggcacctctgctggtcagcaggacatcgcccgcaaggtgttgggtgaggacttccagaaggtcttcagcatcaacaagggttcgatcccggtacgcaacgacatgcttgccgacatgggcaagtatggtttcgatgcctgtgctcaaacctccgccaaggacttcctggctgacgccaaaacgggcggcctgcaaccgagcatggcgcacaacatggccaccacgctggccgtgcagggcgcgttcttcgatgtggtgaccaactacatcaacgaccccaaggccgacccggccgatgcggcgaagaagctggcggcggctatcaaggctgcccagtaacggctgttcgcgggcacgcccgctcccagatgggccctgctcaccactgttcctggtcatgacgccggacctgtgggagcgggcttgcccgcgaagcggtgcgtagcaccgccactacctcgggatccaatacatggacttgttgctggctctgctgccggctttgttctggggttcgatcgtgctgttcaacgtcaaactgggcggcggcccctacagccagaccctgggcaccaccatcggggctctgatcgtctcgattgtcatttatttctttgtgcagccagtgctgagcctgcgcatcttcatcgtcggcatcgtgagcggcctgttctggagcctgggtcaggccaaccagctgaagtcgatccagttgatgggggtaagcaagaccatgccaatttccaccggcatgcagttggtgtcgacctcgctgtttggcgtgatcgtgtttcgggaatggtcgaccccgatcgccatcacgctcggggtcctcgccctgatcttcatcatcgtgggcatcatcctcacctcgctggaggataaaaacgacaaaaaagaaggggagccgtccaacctgaaaaagggtatcctgatcctgctggtgtcgaccctcggctacctggtgtatgtagtggtggcccgcctgttcaacgtctccggttggtcggcgttgctgccgcaggcaatcggcatggtggtgggcggcctggtcttgacctaccggcacaaaccattcaacaagtatgcgattcgtaatatcttgccgggcctgatctgggctggcggcaacatgttcctgtttattagccagccgcgcgtgggtgtcgccacctcctttagcctgagccagatgggcatcgtaatctcgaccttgggcggcatctttatcctccgtgagaagaagacgaagcggcagctgatcgcgatcgccatcggcatcatcttgatcatcgcggccgccgtgttcctgggcatcgccaagaccaacagctagctcgtctacaccatcaataagaaaaagaggacgcaaagggatggaacagcgcaaacgcatcaagacactgggatcgttggccttgcttgcacttgtaggcagcagcggtacacaggctgccgaggctttttccagcgaatccaaatggatgaccggcgactggggcggcacccggaccgagctgctggacaagggctatgacttcaccctcgattatgtgggtgaggtggctggcaacctgcatggcggctacaacgacgacaagacggcacgctacagcgaccagttcgccctcggcgcgcatctggacttgcagaagatactgggctggcatgatgccgagttcaagctggcaatcaccgagcgaagcggtcgcaacctgtccaacgaccgcatcagcgacccgcgcgccgggcagttcagctcggtgcaggaggtgtggggccgtggccagacctggcgcctgacccagatgtggatcaagcagaagtacttcgacggcgcgctggacgtgaaatttggccgttttggcgagggcgaggacttcaacagcttcccttgcgacttccagaacctggccttctgcggctcgcaggtgggcaactgggtgggcggcatctggtacaactggccggtcagccagtgggcgctgcgggtgaagtacaacatcacgccggagttcttcgtacaggtcggggccttcgagcagaacccttccaacctggaaaccggcaacggcttcaagctcagcggcagtggtaccaagggggcgatcttgccggtggaagcggtgtggtcgcccaaggtcaatggcctgccgggcgagtaccgcctgggttactactacagcacggccaaggctgacgatgtgtacgacgacgtcaacggcaacccgcaggcgctgacaggtgaagccttcaagtcgcactccagcaagcacggatggtgggtggtggcgcagcagcaggtcactgcccatggcggcgacgtcaaccggggcctcagcctgttcgccaacttcaccgtgcacgacaaggccaccaacgtggtcgacaactaccagcaggtggggctggtctacaaaggcgctttcgacgcccggcccaaggatgacatcggcttcggcgtggcgcgtattcatgtgaatgacgacgtgaagaagcgcgccgaactgctcaacgcacagagcggcatcaacgattacgacaaccctggtttcgtgccgctgcagcgtaccgaatacaacgcagagctctactacggcttccacgttaccaactggctgaccgtgaggcccaacctgcagtacatcaagagccctggcggggtggacgaggtggataacgcgctggtcgctggcttgaagattcagtcgtcattctgaggcaacttgttgtaaatttacgccaatccattttcggttcccggcacccactggtctgcagtgggtgttggggataggccgagacagcgctatctcaaacaacaagagctctgaaccatgcccgaacatccgctccatcgcttcttctcctcgcagcggccccggccgacattcgagtgggagcgttatcagcagcgcgatgtcctgatcatcgaccacccccgttgccaggcggtgttcagccgccagggcgcgcagttgctgcactttcaaccagcgggtgaacggccgtggctgtggt

SEQ ID NO: 68 - DNA, 42 bp, Artificial Sequence
  primer
tacctcgggatccaatacatggaaaacttgatcggctatgtg

SEQ ID NO: 69 - DNA, 24 bp, Artificial Sequence
  primer
tcaggtttggttgccctcggtcag

SEQ ID NO: 70 - DNA, 47 bp, Artificial Sequence
  primer
ccgagggcaaccaaacctgactcgtctacaccatcaataagaaaaag

SEQ ID NO: 71 - DNA, 276 bp, Artificial Sequence
  PCR product
tacctcgggatccaatacatggaaaacttgatcggctatgtggccgcctttctgacgaccgtgtccttcctgcctcaggtgctgcgcgtcgtcatgaccaagcagacccgcgacatcagccgcaacatgtatatcatgttctttctgggggtcgtgctgtggttcgtgtacggcatcctgcggagcgacctccctatcatcttggccaacgtggtgaccctcttcttcgtgaccatcattctgtactataaactgaccgagggcaaccaaacctga

SEQ ID NO: 72 - DNA, 710 bp, Artificial Sequence
  PCR product
ccgagggcaaccaaacctgactcgtctacaccatcaataagaaaaagaggacgcaaagggatggaacagcgcaaacgcatcaagacactgggatcgttggccttgcttgcacttgtaggcagcagcggtacacaggctgccgaggctttttccagcgaatccaaatggatgaccggcgactggggcggcacccggaccgagctgctggacaagggctatgacttcaccctcgattatgtgggtgaggtggctggcaacctgcatggcggctacaacgacgacaagacggcacgctacagcgaccagttcgccctcggcgcgcatctggacttgcagaagatactgggctggcatgatgccgagttcaagctggcaatcaccgagcgaagcggtcgcaacctgtccaacgaccgcatcagcgacccgcgcgccgggcagttcagctcggtgcaggaggtgtggggccgtggccagacctggcgcctgacccagatgtggatcaagcagaagtacttcgacggcgcgctggacgtgaaatttggccgttttggcgagggcgaggacttcaacagcttcccttgcgacttccagaacctggccttctgcggctcgcaggtgggcaactgggtgggcggcatctggtacaactggccggtcagccagtgggcgctgcgggtgaagtacaacatcacgcctgcaggcatgcaagc

SEQ ID NO: 73 - DNA, 6398 bp, Artificial Sequence
  vector
atggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgggctcgatcccctcgcgagttggttcagctgctgcctgaggctggacgacctcgcggagttctaccggcagtgcaaatccgtcggcatccaggaaaccagcagcggctatccgcgcatccatgcccccgaactgcaggagtggggaggcacgatggccgctttggtcccggccgccaaggtcattaacggcaaggccggcatgcagatcatgggcgactgggccaagagcgaatggaccctggcgaagaaaactgctggcaaggattaccagtgcgtgccgttccccggtactgacaagtccttcctgtacaacatcgactcgttggtggtgttcaagcagaacaacgctggcacctctgctggtcagcaggacatcgcccgcaaggtgttgggtgaggacttccagaaggtcttcagcatcaacaagggttcgatcccggtacgcaacgacatgcttgccgacatgggcaagtatggtttcgatgcctgtgctcaaacctccgccaaggacttcctggctgacgccaaaacgggcggcctgcaaccgagcatggcgcacaacatggccaccacgctggccgtgcagggcgcgttcttcgatgtggtgaccaactacatcaacgaccccaaggccgacccggccgatgcggcgaagaagctggcggcggctatcaaggctgcccagtaacggctgttcgcgggcacgcccgctcccagatgggccctgctcaccactgttcctggtcatgacgccggacctgtgggagcgggcttgcccgcgaagcggtgcgtagcaccgccactacctcgggatccaatacatggaaaacttgatcggctatgtggccgcctttctgacgaccgtgtccttcctgcctcaggtgctgcgcgtcgtcatgaccaagcagacccgcgacatcagccgcaacatgtatatcatgttctttctgggggtcgtgctgtggttcgtgtacggcatcctgcggagcgacctccctatcatcttggccaacgtggtgaccctcttcttcgtgaccatcattctgtactataaactgaccgagggcaaccaaacctgactcgtctacaccatcaataagaaaaagaggacgcaaagggatggaacagcgcaaacgcatcaagacactgggatcgttggccttgcttgcacttgtaggcagcagcggtacacaggctgccgaggctttttccagcgaatccaaatggatgaccggcgactggggcggcacccggaccgagctgctggacaagggctatgacttcaccctcgattatgtgggtgaggtggctggcaacctgcatggcggctacaacgacgacaagacggcacgctacagcgaccagttcgccctcggcgcgcatctggacttgcagaagatactgggctggcatgatgccgagttcaagctggcaatcaccgagcgaagcggtcgcaacctgtccaacgaccgcatcagcgacccgcgcgccgggcagttcagctcggtgcaggaggtgtggggccgtggccagacctggcgcctgacccagatgtggatcaagcagaagtacttcgacggcgcgctggacgtgaaatttggccgttttggcgagggcgaggacttcaacagcttcccttgcgacttccagaacctggccttctgcggctcgcaggtgggcaactgggtgggcggcatctggtacaactggccggtcagccagtgggcgctgcgggtgaagtacaacatcacgcctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctttccttcttcactgtcccttctagaacgttctagaagggacagtgaagaaggaacacccgctcgcgggtgggcctactctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggtcaggcgtccttttgcttggtgccgaagatcttgtcaccggcatcacccaggcctggcacgatgtagccgtgctcgttcaggcgctggtcgatcgaggcggtgtagatcttcacgtccgggtgggctttttccaccacctcgatgccttctggtgcggcgaccagcaccatggcgcgaatctctttgcagccggcctttttcagcaggtcgatggtggcaaccatcgagccgccggtggccagcatcgggtcgatgatcagggccaggcgctggttgatgtccggcgcgagcttttccagataggtgtgggcttcgagggtttcttcgtttcgggcaacgccgacggcgctgaccttggcccccgggatcaggctgagcacgccgtcgagcatgccgatgccggcgcgcaggatcggtactacggtgatcttcttgccggcgattttttcaaccgagaccttgccacaccagccgtcgatctcgtaggtttcgaggggcaggtcctgggtggcttcatacgtcaggagcgcgccgacttcctgggcgagttcgcgaaaattcttggtgctgatatcggcacggcgcatcaggccaagcttgtggcggatcagcggatggcggatctcacgagtgggcataggggagggctccgaaaggcgggcaaaaaaaccgcgctagattaatctattcagcctgtgctgtcgtctggtcattctggacgttagtccataaatgcttgatctgtgacgagcggatgcgtacctttgcccgcttttccaaaatgctagccggctacgtatcgataagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctg

SEQ ID NO: 74 - DNA, 3460 bp, Artificial Sequence
  knockout construct
gcatcgcttcgttacctgacgaggcgggcacgctgtttagacttcatgctacgcaagcgatcgacttgcacggcataacaacaagaaaggtgctccgatgaattccacgcttcgtctcgctgccgcaatttcctttgcctcgttaatccccttgggtgcccaggctgctgacgccaaaggcagtgtcgaagtggtgcactggtggacctccggtggtgaaaaagcggcggtcgatgtgctcaaggcccaggtcgaaaaagacggcttcatctggaaggacggcgccgtcgccggcggcggtggtgcaacggccatgaccgtgctcaaaagccgcgcagtggccggcaacccgccgggcgtcgcgcagatcaaaggcccggacatccaggactgggcggccaccggcctgctcgacgccgatgtgctcaaggatgtggccaaggaaggaaagtgggactcgctgctcgacaagaaagtggccgacaccgtgaagtacgacggtgactacgttgccgtaccggtgaatatccaccgcatcaactggctgtggatcaaccccgaggtgttcaaaaaagccggcatcgacaaggcgcccaccaccctcgacgaattctacgccgccgccgacaagctcaaggctgccggtttcatcccgctcgcccatggtgggcaaccctggcaggacagcaccgtgttcgaaagcgtggtgctgtcggtgatgggcgtcgatggctacaagaaggccttggtcgacctcgacagcgcaacgctgaccgggccgcagatggtcaaggcgctgaccgagttgaagaaagtcgccacctacatggacccggacggcaagggccaggactggaacctggaagccgccaaggtcattaacggcaaggccggcatgcagatcatgggcgactgggccaagagcgaatggaccctggcgaagaaaactgctggcaaggattaccagtgcgtgccgttccccggtactgacaagtccttcctgtacaacatcgactcgttggtggtgttcaagcagaacaacgctggcacctctgctggtcagcaggacatcgcccgcaaggtgttgggtgaggacttccagaaggtcttcagcatcaacaagggttcgatcccggtacgcaacgacatgcttgccgacatgggcaagtatggtttcgatgcctgtgctcaaacctccgccaaggacttcctggctgacgccaaaacgggcggcctgcaaccgagcatggcgcacaacatggccaccacgctggccgtgcagggcgcgttcttcgatgtggtgaccaactacatcaacgaccccaaggccgacccggccgatgcggcgaagaagctggcggcggctatcaaggctgcccagtaacggctgttcgcgggcacgcccgctcccagatgggccctgctcaccactgttcctggtcatgacgccggacctgtgggagcgggcttgcccgcgaagcggtgcgtagcaccgccactacctcgggatccaatacatggaaaacttgatcggctatgtggccgcctttctgacgaccgtgtccttcctgcctcaggtgctgcgcgtcgtcatgaccaagcagacccgcgacatcagccgcaacatgtatatcatgttctttctgggggtcgtgctgtggttcgtgtacggcatcctgcggagcgacctccctatcatcttggccaacgtggtgaccctcttcttcgtgaccatcattctgtactataaactgaccgagggcaaccaaacctgactcgtctacaccatcaataagaaaaagaggacgcaaagggatggaacagcgcaaacgcatcaagacactgggatcgttggccttgcttgcacttgtaggcagcagcggtacacaggctgccgaggctttttccagcgaatccaaatggatgaccggcgactggggcggcacccggaccgagctgctggacaagggctatgacttcaccctcgattatgtgggtgaggtggctggcaacctgcatggcggctacaacgacgacaagacggcacgctacagcgaccagttcgccctcggcgcgcatctggacttgcagaagatactgggctggcatgatgccgagttcaagctggcaatcaccgagcgaagcggtcgcaacctgtccaacgaccgcatcagcgacccgcgcgccgggcagttcagctcggtgcaggaggtgtggggccgtggccagacctggcgcctgacccagatgtggatcaagcagaagtacttcgacggcgcgctggacgtgaaatttggccgttttggcgagggcgaggacttcaacagcttcccttgcgacttccagaacctggccttctgcggctcgcaggtgggcaactgggtgggcggcatctggtacaactggccggtcagccagtgggcgctgcgggtgaagtacaacatcacgccggagttcttcgtacaggtcggggccttcgagcagaacccttccaacctggaaaccggcaacggcttcaagctcagcggcagtggtaccaagggggcgatcttgccggtggaagcggtgtggtcgcccaaggtcaatggcctgccgggcgagtaccgcctgggttactactacagcacggccaaggctgacgatgtgtacgacgacgtcaacggcaacccgcaggcgctgacaggtgaagccttcaagtcgcactccagcaagcacggatggtgggtggtggcgcagcagcaggtcactgcccatggcggcgacgtcaaccggggcctcagcctgttcgccaacttcaccgtgcacgacaaggccaccaacgtggtcgacaactaccagcaggtggggctggtctacaaaggcgctttcgacgcccggcccaaggatgacatcggcttcggcgtggcgcgtattcatgtgaatgacgacgtgaagaagcgcgccgaactgctcaacgcacagagcggcatcaacgattacgacaaccctggtttcgtgccgctgcagcgtaccgaatacaacgcagagctctactacggcttccacgttaccaactggctgaccgtgaggcccaacctgcagtacatcaagagccctggcggggtggacgaggtggataacgcgctggtcgctggcttgaagattcagtcgtcattctgaggcaacttgttgtaaatttacgccaatccattttcggttcccggcacccactggtctgcagtgggtgttggggataggccgagacagcgctatctcaaacaacaagagctctgaaccatgcccgaacatccgctccatcgcttcttctcctcgcagcggccccggccgacattcgagtgggagcgttatcagcagcgcgatgtcctgatcatcgaccacccccgttgccaggcggtgttcagccgccagggcgcgcagttgctgcactttcaaccagcgggtgaacggccgtggctgtggt